SEQUENCE LISTING (30 sequences)

SEQ ID NO: 1 - protein, 15 aa, synthetic construct
PVSKMRMATPLLMQA

SEQ ID NO: 2-3 - skipped (2 entries)

SEQ ID NO: 4 - protein, 15 aa, synthetic construct
  VARIANT 1..15: note = This sequence may encompass 4-15 residues
HHHHHHHHHHHHHHH

SEQ ID NO: 5 - protein, 8 aa, synthetic construct
HHHHHHHH

SEQ ID NO: 6 - protein, 16 aa, Thosea asigna virus
EGRGSLTCGDVENPGP

SEQ ID NO: 7 - protein, 17 aa, Teschovirus A
ATNFSLKQAGDVENPGP

SEQ ID NO: 8 - protein, 19 aa, Equine rhinitis A virus
QCTNYALKLAGDVESNPGP

SEQ ID NO: 9 - protein, 21 aa, Foot-and-mouth disease virus
VKQTLNFDLKLAGDVESNPGP

SEQ ID NO: 10 - protein, 9 aa, Homo sapiens
RLLIEDPYL

SEQ ID NO: 11 - protein, 9 aa, Homo sapiens
NLPGFLPAL

SEQ ID NO: 12 - protein, 9 aa, Homo sapiens
HSAINEVVT

SEQ ID NO: 13 - protein, 9 aa, Homo sapiens
RLAQGSSAV

SEQ ID NO: 14 - protein, 9 aa, Homo sapiens
HLMEIGESL

SEQ ID NO: 15 - protein, 9 aa, Homo sapiens
VIDSTIQSV

SEQ ID NO: 16 - protein, 9 aa, Homo sapiens
HLYSGAVTI

SEQ ID NO: 17 - protein, 9 aa, Homo sapiens
MRIATPLLM

SEQ ID NO: 18 - protein, 9 aa, Homo sapiens
YHGDDGHLF

SEQ ID NO: 19 - protein, 10 aa, Homo sapiens
THSQLIVVSL

SEQ ID NO: 20 - protein, 8 aa, Homo sapiens
ILDVNVNL

SEQ ID NO: 21 - protein, 9 aa, Homo sapiens
VAFGHLLAI

SEQ ID NO: 22 - protein, 9 aa, Homo sapiens
VHDAAQEGF

SEQ ID NO: 23 - protein, 10 aa, Homo sapiens
EHVPPYDVVL

SEQ ID NO: 24 - protein, 9 aa, Homo sapiens
QYQKVLVLF

SEQ ID NO: 25 - protein, 9 aa, Homo sapiens
SHQSFQHLL

SEQ ID NO: 26 - protein, 10 aa, Homo sapiens
RVPFAFPLHR

SEQ ID NO: 27 - protein, 9 aa, Homo sapiens
SYVTSHQGF

SEQ ID NO: 28 - protein, 9 aa, Homo sapiens
MLFPTSAQK

SEQ ID NO: 29 - protein, 9 aa, Homo sapiens
HSAINEVVT

SEQ ID NO: 30 - protein, 8 aa, Homo sapiens
VIDSTIQV

(19) United States
(12) Patent Application Publication
(10) Pub. No.: US 2026/0120799 A1
(43) Pub. Date: Apr. 30, 2026

(54) METHODS AND SYSTEMS FOR PREDICTION OF HLA EPITOPES

(71) Applicant: BioNTech US Inc., Cambridge, MA (US)

(72) Inventors: Theresa A. Addona, Needham, MA (US); Daniel Abram Rothenberg, Melrose, MA (US); Michael Steven Rooney, Boston, MA (US)

(21) Appl. No.: 19/102,057

(22) PCT Filed: Aug. 11, 2023
(86) PCT No.: PCT/US2023/072085

§ 371 Date: Feb. 7, 2025

Related U.S. Application Data
(60) Provisional application No. 63/397,669, filed on Aug. 12, 2022.

(51) Int. Cl. G16B 30/00; G16B 40/20; G16B 40/30

(52) U.S. Cl. CPC G16B 30/00; G16B 40/20; G16B 40/30

(57) ABSTRACT

Methods for preparing a personalized cancer vaccine and a method to train a machine-learning HLA-peptide prediction model.

### CROSS-REFERENCE

This application is a 371 U.S. National Phase Application of International Application No. PCT/US2023/072085, filed on Aug. 11, 2023, which claims the benefit of U.S. Provisional Application No. 63/397,669, filed on Aug. 12, 2022, each of which is incorporated herein by reference in its entirety.

### SEQUENCE LISTING

The instant application contains a Sequence Listing which has been submitted electronically in XML file format and is hereby incorporated by reference in its entirety. Said XML copy, created on Feb. 14, 2025, is named 50401-770_601_SL.xml and is 25,920 bytes in size.

### BACKGROUND

The major histocompatibility complex (MHC) is a gene complex encoding human leukocyte antigen (HLA) genes. HLA genes are expressed as protein heterodimers that are displayed on the surface of human cells to circulating T cells. HLA genes are highly polymorphic, allowing them to fine-tune the adaptive immune system. Adaptive immune responses rely, in part, on the ability of T cells to identify and eliminate cells that display disease-associated peptide antigens bound to human leukocyte antigen (HLA) heterodimers.

In humans, endogenous and exogenous proteins can be processed into peptides by the proteasome and by cytosolic and endosomal/lysosomal proteases and peptidases and presented by two classes of cell surface proteins encoded by MHC genes. These cell surface proteins are referred to as human leukocyte antigens (HLA class I and class II), and the group of peptides that bind them and elicit immune responses are termed HLA epitopes. HLA epitopes are a key component that enables the immune system to detect danger signals, such as pathogen infection and transformation of self. Typically, CD8+ T cells recognize MHC class I epitopes displayed on antigen presenting cells (APCs), such as dendritic cells and macrophages and CD4+ T cells recognize class II MHC (HLA-DR, HLA-DQ, and HLA-DP) epitopes displayed on APCs. The endogenous processing and presentation of HLA epitopes is a complex procedure and involves a variety of chaperones and a subset of enzymes. HLA peptide presentation can activate cytotoxic T cells and helper T cells, subsequently promoting B cell differentiation and antibody production as well as CTL responses.

Understanding the peptide-binding preferences of every HLA class I or class II molecules is the key to successfully predicting which cancer or tumor-specific antigens are likely to elicit the cancer or tumor-specific T cell responses. There is a need for methods of identifying and isolating specific HLA class I or class II-associated peptides (e.g., neoantigen peptides). Such methodology and isolated molecules are useful, e.g., for the development of therapeutics, including but not limited to, immune based therapeutics.

### SUMMARY

Provided herein is a method of identifying peptide sequences as being presented by at least one of the one or more proteins encoded by an HLA allele of a cell of the subject comprising: (a) inputting amino acid sequence information of a set of candidate peptide sequences expressed by cancer cells of a single human subject, using a computer processor, into a trained machine learning HLA-peptide presentation prediction model, to generate a plurality of presentation predictions, wherein each presentation prediction of the plurality of presentation predictions is indicative of a presentation likelihood that a peptide sequence of the set of candidate peptide sequences is presented by an MHC protein of the single human subject; wherein the trained machine learning HLA-peptide presentation prediction model comprises: (i) a plurality of parameters, wherein the plurality of parameters are based on training data from training cells expressing an MHC protein, wherein the training data comprises a plurality of training peptide sequences and epitope presentation quantification information, wherein the epitope presentation quantification information comprises the amount of one or more of the training peptide sequences of the plurality that was presented by an MHC protein; and (ii) a function representing a relation between the amino acid sequence information received as input and the presentation likelihood generated as an output based on the amino acid sequence information and the plurality of parameters; and (b) identifying, based at least on the plurality of presentation predictions, a peptide sequence of the plurality of peptide sequences of the set of candidate peptide sequences as being presented by at least one of the one or more proteins encoded by an HLA allele of a cell of the subject.

Provided herein is a method of selecting peptide sequences comprising: (a) inputting amino acid sequence information of a set of candidate peptide sequences expressed by cancer cells of a single human subject, using a computer processor, into a trained machine learning HLA-peptide presentation prediction model, to generate a plurality of presentation predictions, wherein each presentation prediction of the plurality of presentation predictions is indicative of a presentation likelihood that a peptide sequence of the set of candidate peptide sequences is presented by an MHC protein of the single human subject; wherein the trained machine learning HLA-peptide presentation prediction model comprises: (i) a plurality of parameters, wherein the plurality of parameters are based on training data from training cells expressing an MHC protein, wherein the training data comprises a plurality of training peptide sequences and epitope presentation quantification information, wherein the epitope presentation quantification information comprises the amount of one or more of the training peptide sequences of the plurality that was presented by an MHC protein; and (ii) a function representing a relation between the amino acid sequence information received as input and the presentation likelihood generated as an output based on the amino acid sequence information and the plurality of parameters; and (b) selecting, based at least on the plurality of presentation predictions, a subset of peptide sequences of the set of candidate peptide sequences to generate a set of selected peptide sequences.

Provided herein is a method of treating cancer in a human subject in need thereof comprising: (a) inputting amino acid sequence information of a set of candidate peptide sequences expressed by cancer cells of a single human subject, using a computer processor, into a trained machine learning HLA-peptide presentation prediction model, to generate a plurality of presentation predictions, wherein each presentation prediction of the plurality of presentation predictions is indicative of a presentation likelihood that a peptide sequence of the set of candidate peptide sequences is presented by an MHC protein of the single human subject; wherein the trained machine learning HLA-peptide presentation prediction model comprises: (i) a plurality of parameters, wherein the plurality of parameters are based on training data from training cells expressing an MHC protein, wherein the training data comprises a plurality of training peptide sequences and epitope presentation quantification information, wherein the epitope presentation quantification information comprises the amount of one or more of the training peptide sequences of the plurality that was presented by an MHC protein; and (ii) a function representing a relation between the amino acid sequence information received as input and the presentation likelihood generated as an output based on the amino acid sequence information and the plurality of parameters; (b) selecting or identifying, based at least on the plurality of presentation predictions, a subset of peptide sequences of the set of candidate peptide sequences to generate a set of selected or identified peptide sequences; and (c) administering to the single human subject a pharmaceutical composition comprising: (i) a polypeptide with one or more of the selected peptide sequences, (ii) a polynucleotide encoding the polypeptide of (i); (iii) APCs comprising (i) or (ii), or (iv) T cells comprising a T cell receptor (TCR) specific for an MHC protein of the single human subject in complex with one or more of the peptide sequences selected or identified in (b).

In some embodiments, the plurality of parameters are based on training data from training cells expressing an MHC protein of the single human subject.

In some embodiments, each training peptide sequence of the plurality is associated with an MHC protein.

In some embodiments, the training data comprises an identity of the MHC protein associated with each training peptide sequence of the plurality.

In some embodiments, the training data comprises an observation by mass spectrometry that one or more of the training peptide sequences of the plurality was presented by an MHC protein.

In some embodiments, the MHC protein of the single human subject is a class I MHC protein.

In some embodiments, the plurality of candidate peptide sequences expressed by cancer cells of a single human subject are identified by comparing whole genome or whole exome sequence information from the cancer cells of the single human subject to whole genome or whole exome sequence information from non-cancer cells of the single human subject, and identifying nucleic acid sequences unique to the cancer cells and not present in the non-cancer cells.

In some embodiments, each candidate sequence of the plurality of candidate peptide sequences comprises a cancer specific mutation.

In some embodiments, the trained machine learning HLA-peptide presentation prediction model having a peptide presentation prediction value (PPV) of at least 0.2 according to a presentation PPV determination method.

In some embodiments, the presentation PPV determination method comprises inputting amino acid sequence information of a plurality of test peptide sequences into the trained machine learning HLA-peptide presentation prediction model to generate a plurality of test presentation predictions, each test presentation prediction indicative of a likelihood that the one or more proteins encoded by an HLA allele can present a given test peptide sequence of the plurality of test peptide sequences, wherein the plurality of test peptide sequences comprises at least 500 test peptide sequences comprising: (i) at least one hit peptide sequence identified by mass spectrometry to be presented by an HLA protein expressed in cells, and (ii) at least 499 decoy peptide sequences contained within a protein encoded by a genome of an organism, wherein the organism and the subject are the same species.

In some embodiments, the plurality of test peptide sequences comprises a ratio of 1:499 of the at least one hit peptide sequence to the at least 499 decoy peptide sequences and a top 0.2% of the plurality of test peptide sequences are predicted to be presented by the HLA protein expressed in cells by the trained machine learning HLA-peptide presentation prediction model.

In some embodiments, (i) the at least one hit peptide sequence comprises at least 10 hit peptide sequences, and (ii) the at least 499 decoy peptide sequences comprise at least 4,990 decoy peptide sequences.

In some embodiments, the amount of one or more of the training peptide sequences of the plurality that was presented by an MHC protein comprises the number of copies of the one or more of the training peptide sequences of the plurality that was presented by an MHC protein.

In some embodiments, the amount of one or more of the training peptide sequences of the plurality that was presented by an MHC protein comprises the number of copies per cell of the one or more of the training peptide sequences of the plurality that was presented by an MHC protein.

In some embodiments, the amount of one or more of the training peptide sequences of the plurality that was presented by an MHC protein comprises the absolute quantity, the number of molecules, density, concentration, absolute quantity per cell, the number of molecules per cell, density per cell, or concentration in a cell of the one or more of the training peptide sequences of the plurality that was presented by an MHC protein.

In some embodiments, the amount of one or more of the training peptide sequences of the plurality that was presented by an MHC protein is based on a number of mass spectrometry observances, spectral counting, area under the curve (AUC), intensity-based absolute quantification (iBAQ), label free quantification (LFQ), isotope dilution mass spectrometry, isobaric mass tagging, stable isotope labeling, and/or mass spectrometry peak intensity.

In some embodiments, the amount of one or more of the training peptide sequences of the plurality that was presented by an MHC protein is obtained from quantitative mass spectrometry.

In some embodiments, the epitope presentation quantification information is obtained from internal standard-parallel reaction monitoring (IS-PRM) mass spectrometry.

In some embodiments, the epitope presentation quantification information is obtained from a xenograft sample.

In some embodiments, the xenograft sample is a patient-derived xenograft (PDX) sample.

Also provided herein is a method of selecting peptide sequences comprising: (a) inputting amino acid sequence information of a set of candidate peptide sequences expressed by cancer cells of a single human subject, using a computer processor, into a trained machine learning HLA-peptide antigen-specific T cell prediction model, to generate a plurality of antigen-specific T cell predictions, wherein each antigen-specific T cell prediction of the plurality of antigen-specific T cell predictions is indicative of a likelihood that an MHC complex comprising an MHC protein of the single human subject and a peptide sequence of the set of candidate peptide sequences stimulates a T cell to be specific to a peptide sequence of the set of candidate peptide sequences; wherein the trained machine learning HLA-peptide cytotoxic T cell prediction model comprises: (i) a plurality of parameters, wherein the plurality of parameters are based on training data from training cells expressing an MHC protein, wherein the training data comprises a plurality of training peptide sequences and epitope presentation quantification information, wherein the epitope presentation quantification information comprises the amount of one or more of the training peptide sequences of the plurality that was presented by an MHC protein; and (ii) a function representing a relation between the amino acid sequence information received as input and the likelihood that a T cell specific to a peptide sequence of the set of candidate peptide sequences would be generated as an output based on the amino acid sequence information and the plurality of parameters; and (b) selecting, based at least on the plurality of antigen-specific T cell predictions, a subset of peptide sequences of the set of candidate peptide sequences to generate a set of selected peptide sequences.

In some embodiments, each antigen-specific T cell prediction of the plurality of antigen-specific T cell predictions is indicative of a likelihood that an MHC complex comprising an MHC protein of the single human subject and a peptide sequence of the set of candidate peptide sequences stimulates a T cell to be specific to a neoantigen peptide sequence of the set of candidate peptide sequences.

In some embodiments, the function is a function representing a relation between the amino acid sequence information received as input and the likelihood that a T cell specific to a neoantigen peptide sequence of the set of candidate peptide sequences would be generated as an output based on the amino acid sequence information and the plurality of parameters.

In some embodiments, each antigen-specific T cell prediction of the plurality of antigen-specific T cell predictions is indicative of a likelihood that an MHC complex comprising an MHC protein of the single human subject and a peptide sequence of the set of candidate peptide sequences stimulates a T cell to be cytotoxic.

In some embodiments, the function is a function representing a relation between the amino acid sequence information received as input and the likelihood that a cytotoxic T cell would be generated as an output based on the amino acid sequence information and the plurality of parameters.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “FIG.” herein), of which:

FIG. 1A depicts data showing evaluation results using hold-out partition of Mono-Allelic Dataset.

FIG. 1B depicts data showing predictor performance in Ovarian Tumors Profiled by MS.

FIG. 1C shows RECON presentation score.

FIG. 2A depicts a diagram showing an exemplary workflow for making of patient derived xenografts for targeted MS.

FIG. 2B depicts a diagram showing sequence overlap of non-synonymous mutations.

FIG. 3A depicts an exemplary workflow of a method of validation of predicted neoantigens using internal standard triggered parallel reaction monitoring. Figure discloses SEQ ID NOs: 10, 19, 14, 30, 10, 19, 14, and 30, respectively, in order of appearance.

FIG. 3B shows data of respective validation of predicted neoantigens by parallel reaction monitoring. Figure discloses SEQ ID NOs: 10, 19, 14, and 30, respectively, in order of appearance.

FIG. 4 shows RECON® presentation scores across epitopes targeted by MS.

FIG. 5A shows an exemplary workflow for quantitation of predicted neoantigens.

FIG. 5B shows quantitation of predicted neoantigens. Figure discloses SEQ ID NOs: 10-11, respectively, in order of appearance.

FIG. 5C shows quantitation of predicted neoantigens. Figure discloses SEQ ID NOs: 11 and 10, respectively, in order of appearance.

FIG. 6A shows T cell responses to observed neoantigens. Figure discloses SEQ ID NOs: 11, 10, 10, and 10, respectively, in order of appearance.

FIG. 6B shows immune monitoring correlation data. Figure discloses SEQ ID NOs: 10-11, respectively, in order of appearance.

FIG. 7 shows binding affinity correlation data. Figure discloses SEQ ID NOs: 14, 16, 11, 13, 10, and 15, respectively, in order of appearance.

FIG. 8 shows clinical outcome data.

### DETAILED DESCRIPTION

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the disclosure can also be implemented in a single embodiment.

The methods and compositions described herein find uses in a wide range of applications. For example, the methods and compositions described herein can be used to identify immunogenic antigen peptides and can be used to develop drugs, such as personalized medicine drugs, and isolation and characterization of antigen-specific T cells.

The methods disclosed herein may comprise generating LC-MS/MS allelic data for the training of allele-specific machine learning methods for epitope prediction. Such methods may comprise increasing LC-MS/MS data quality utilizing a set of quality metrics to stringently remove false positives that increases the performance of a prediction model; identifying allele-specific HLA class I or class II binding cores from HLA-ligandome LC-MS/MS datasets; utilizing machine learning algorithms to improve HLA class I or class II-ligand and epitope prediction; and/or identifying biological variables that impact HLA class I or class II-ligand presentation and improve HLA class I or class II epitope prediction, such as gene expression, cleavability, gene bias, cellular localization, and secondary structure.

Provided herein is a method comprising: (a) processing amino acid information of a plurality of candidate peptide sequences using a machine learning HLA peptide presentation prediction model to generate a plurality of presentation predictions, wherein each candidate peptide sequence of the plurality of candidate peptide sequences is encoded by a genome or exome of a subject, wherein the plurality of presentation predictions comprises an HLA presentation prediction for each of the plurality of candidate peptide sequences, wherein each HLA presentation prediction is indicative of a likelihood that one or more proteins encoded by a class I or class II HLA allele of a cell of the subject can present a given candidate peptide sequence of the plurality of candidate peptide sequences, wherein the machine learning HLA peptide presentation prediction model is trained using training data comprising sequence information of sequences of training peptides identified by mass spectrometry to be presented by an HLA protein expressed in training cells; and (b) identifying, based at least on the plurality of presentation predictions, a peptide sequence of the plurality of peptide sequences as being presented by at least one of the one or more proteins encoded by a class I or class II HLA allele of a cell of the subject; wherein the machine learning HLA peptide presentation prediction model has a positive predictive value (PPV) of at least 0.07 according to a presentation PPV determination method.

Provided herein is a method comprising: (a) processing amino acid information of a plurality of peptide sequences of encoded by a genome or exome of a subject using a machine learning HLA peptide binding prediction model to generate a plurality of binding predictions, wherein the plurality of binding predictions comprises an HLA binding prediction for each of the plurality of candidate peptide sequences, each binding prediction indicative of a likelihood that one or more proteins encoded by a class II HLA allele of a cell of the subject binds to a given candidate peptide sequence of the plurality of candidate peptide sequences, wherein the machine learning HLA peptide binding prediction model is trained using training data comprising sequence information of sequences of peptides identified to bind to an HLA class I or class II protein or an HLA class I or class II protein analog; and (b) identifying, based at least on the plurality of binding predictions, a peptide sequence of the plurality of peptide sequences that has a probability greater than a threshold binding prediction probability value of binding to at least one of the one or more proteins encoded by a class I or class II HLA allele of a cell of the subject; wherein the machine learning HLA peptide binding prediction model has a positive predictive value (PPV) of at least 0.1 according to a binding PPV determination method.

In some embodiments, the machine learning HLA peptide presentation prediction model is trained using training data comprising sequence information of sequences of training peptides identified by mass spectrometry to be presented by an HLA protein expressed in training cells.

In some embodiments, the method comprises ranking, based on the presentation predictions, at least two peptides identified as being presented by at least one of the one or more proteins encoded by a class I or class II HLA allele of a cell of the subject.

In some embodiments, the method comprises selecting one or more peptides of the two or more ranked peptides.

In some embodiments, the method comprises selecting one or more peptides of the plurality that were identified as being presented by at least one of the one or more proteins encoded by a class I or class II HLA allele of a cell of the subject.

In some embodiments, the method comprises selecting one or more peptides of two or more peptides ranked based on the presentation predictions.

In some embodiments, the machine learning HLA peptide presentation prediction model has a positive predictive value (PPV) of at least 0.07 when amino acid information of a plurality of test peptide sequences are processed to generate a plurality of test presentation predictions, each test presentation prediction indicative of a likelihood that the one or more proteins encoded by a class I or class II HLA allele of a cell of the subject can present a given test peptide sequence of the plurality of test peptide sequences, wherein the plurality of test peptide sequences comprises at least 500 test peptide sequences comprising (i) at least one hit peptide sequence identified by mass spectrometry to be presented by an HLA protein expressed in cells and (ii) at least 499 decoy peptide sequences contained within a protein encoded by a genome of an organism, wherein the organism and the subject are the same species, wherein the plurality of test peptide sequences comprises a ratio of 1:499 of the at least one hit peptide sequence to the at least 499 decoy peptide sequences and a top percentage of the plurality of test peptide sequences are predicted to be presented by the HLA protein expressed in cells by the machine learning HLA peptide presentation prediction model.

In some embodiments, the machine learning HLA peptide presentation prediction model has a positive predictive value (PPV) of at least 0.1 when amino acid information of a plurality of test peptide sequences are processed to generate a plurality of test binding predictions, each test binding prediction indicative of a likelihood that the one or more proteins encoded by a class I or class II HLA allele of a cell of the subject binds to a given test peptide sequence of the plurality of test peptide sequences, wherein the plurality of test peptide sequences comprises at least 20 test peptide sequences comprising (i) at least one hit peptide sequence identified by mass spectrometry to be presented by an HLA protein expressed in cells and (ii) at least 19 decoy peptide sequences contained within a protein comprising at least one peptide sequence identified by mass spectrometry to be presented by an HLA protein expressed in cells, such as a single HLA protein expressed in cells (e.g., mono-allelic cells), wherein the plurality of test peptide sequences comprises a ratio of 1:19 of the at least one hit peptide sequence to the at least 19 decoy peptide sequences and a top percentage of the plurality of test peptide sequences are predicted to bind to the HLA protein expressed in cells by the machine learning HLA peptide presentation prediction model.

In some embodiments, no amino acid sequence overlap exist among the at least one hit peptide sequence and the decoy peptide sequences.

In some embodiments, the machine learning HLA peptide presentation prediction model has a positive predictive value (PPV) of at least 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98 or 0.99.

In some embodiments, the at least one hit peptide sequence comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 hit peptide sequences.

In some embodiments, the at least 499 decoy peptide sequences comprises at least 500 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 52500, 55000, 57500, 60000, 62500, 65000, 67500, 70000, 72500, 75000, 77500, 80000, 82500, 85000, 87500, 90000, 92500, 95000, 97500, 100000, 125000, 150000, 175000, 200000, 225000, 250000, 275000, 300000, 325000, 350000, 375000, 400000, 425000, 450000, 475000, 500000, 600000, 700000, 800000, 900000 or 1000000 decoy peptide sequences. One of skill in the art is able to recognize that changing the ratio of hit:decoy changes the PPV.

In some embodiments, the at least 500 test peptide sequences comprises at least 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 52500, 55000, 57500, 60000, 62500, 65000, 67500, 70000, 72500, 75000, 77500, 80000, 82500, 85000, 87500, 90000, 92500, 95000, 97500, 100000, 125000, 150000, 175000, 200000, 225000, 250000, 275000, 300000, 325000, 350000, 375000, 400000, 425000, 450000, 475000, 500000, 600000, 700000, 800000, 900000 or 1000000 test peptide sequences.

In some embodiments, the top percentage is a top 0.20%, 0.30%, 0.40%, 0.50%, 0.60%, 0.70%, 0.80%, 0.90%, 1.00%, 1.10%, 1.20%, 1.30%, 1.40%, 1.50%, 1.60%, 1.70%, 1.80%, 1.90%, 2.00%, 2.10%, 2.20%, 2.30%, 2.40%, 2.50%, 2.60%, 2.70%, 2.80%, 2.90%, 3.00%, 3.10%, 3.20%, 3.30%, 3.40%, 3.50%, 3.60%, 3.70%, 3.80%, 3.90%, 4.00%, 4.10%, 4.20%, 4.30%, 4.40%, 4.50%, 4.60%, 4.70%, 4.80%, 4.90%, 5.00%, 5.10%, 5.20%, 5.30%, 5.40%, 5.50%, 5.60%, 5.70%, 5.80%, 5.90%, 6.00%, 6.10%, 6.20%, 6.30%, 6.40%, 6.50%, 6.60%, 6.70%, 6.80%, 6.90%, 7.00%, 7.10%, 7.20%, 7.30%, 7.40%, 7.50%, 7.60%, 7.70%, 7.80%, 7.90%, 8.00%, 8.10%, 8.20%, 8.30%, 8.40%, 8.50%, 8.60%, 8.70%, 8.80%, 8.90%, 9.00%, 9.10%, 9.20%, 9.30%, 9.40%, 9.50%, 9.60%, 9.70%, 9.80%, 9.90%, 10%, 11%, 12%, 13%, 14%, 15%, 16%, 17%, 18%, 19% or 20%.

In some embodiments, the at least one hit peptide sequence comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 hit peptide sequences.

In some embodiments, the at least 19 decoy peptide sequences comprises at least 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470,480,490, 500 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 52500, 55000, 57500, 60000, 62500, 65000, 67500, 70000, 72500, 75000, 77500, 80000, 82500, 85000, 87500, 90000, 92500, 95000, 97500, 100000, 125000, 150000, 175000, 200000, 225000, 250000, 275000, 300000, 325000, 350000, 375000, 400000, 425000, 450000, 475000, 500000, 600000, 700000, 800000, 900000 or 1000000 decoy peptide sequences.

In some embodiments, the at least 20 test peptide sequences comprises at least wherein the at least 500 test peptide sequences comprises at least 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 52500, 55000, 57500, 60000, 62500, 65000, 67500, 70000, 72500, 75000, 77500, 80000, 82500, 85000, 87500, 90000, 92500, 95000, 97500, 100000, 125000, 150000, 175000, 200000, 225000, 250000, 275000, 300000, 325000, 350000, 375000, 400000, 425000, 450000, 475000, 500000, 600000, 700000, 800000, 900000 or 1000000 test peptide sequences.

In some embodiments, the top percentage is atop 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%.

In some embodiments, the subject is a single subject.

In some embodiments, the subject is a mammal.

In some embodiments, the subject is a human.

In some embodiments, the training cells are cells expressing a single protein encoded by a class I or class II HLA allele of a cell of the subject.

In some embodiments, the training cells are monoallelic HLA cells, or cells expressing an HLA allele with an affinity tag.

In some embodiments, the cell of the subject comprises cancer cells.

In some embodiments, the method is for identifying peptide sequences.

In some embodiments, the method is for selecting peptide sequences.

In some embodiments, the method is for preparing a cancer therapy.

In some embodiments, the method is for preparing a subject-specific cancer therapy.

In some embodiments, the method is for preparing a cancer cell-specific cancer therapy.

In some embodiments, each peptide sequence of the plurality of peptide sequences is associated with a cancer.

In some embodiments, at least one peptide sequence of the plurality of peptide sequences is overexpressed by a cancer cell of the subject.

In some embodiments, each peptide sequence of the plurality of peptide sequences is overexpressed by a cancer cell of the subject.

In some embodiments, at least one peptide sequence of the plurality of peptide sequences is a cancer cell-specific peptide.

In some embodiments, each peptide sequence of the plurality of peptide sequences is a cancer cell-specific peptide.

In some embodiments, each peptide sequence of the plurality of peptide sequences is expressed by a cancer cell of the subject.

In some embodiments, at least one peptide sequence of the plurality of peptide sequences is not encoded by a non-cancer cell of the subject.

In some embodiments, each peptide sequence of the plurality of peptide sequences is not encoded by a non-cancer cell of the subject.

In some embodiments, at least one peptide sequence of the plurality of peptide sequences is not expressed by a non-cancer cell of the subject.

In some embodiments, each peptide sequence of the plurality of peptide sequences is not expressed by a non-cancer cell of the subject.

In some embodiments, the method comprises obtaining the plurality of peptide sequences of the subject.

In some embodiments, the method comprises obtaining a plurality of polynucleotide sequences of the subject.

In some embodiments, the method comprises obtaining a plurality of polynucleotide sequences of the subject that encodes the plurality of peptide sequences encoded by a genome or exome of a subject, or by a pathogen or virus in the subject.

In some embodiments, the method comprises obtaining a plurality of polynucleotide sequences of the subject that encodes the plurality of peptide sequences encoded by a genome or exome of a subject by a computer processor.

In some embodiments, the method comprises obtaining a plurality of polynucleotide sequences of the subject by genomic or exomic sequencing.

In some embodiments, the method comprises obtaining a plurality of polynucleotide sequences of the subject by whole genome sequencing or whole exome sequencing.

In some embodiments, processing comprises processing by a computer processor.

In some embodiments, processing comprises generating a plurality of predictor variables based at least on the amino acid information of the plurality of peptide sequences.

In some embodiments, processing the plurality of predictor variables using the machine-learning HLA-peptide presentation prediction model.

In some embodiments, the one or more proteins encoded by a class I or class II HLA allele of a cell of the subject are one or more proteins encoded by a class I or class II HLA allele that are expressed by the subject.

In some embodiments, the one or more proteins encoded by a class I or class II HLA allele of a cell of the subject are one or more proteins encoded by a class I or class II HLA allele that are expressed by cancer cells of the subject.

In some embodiments, the one or more proteins encoded by a class I or class II HLA allele of a cell of the subject is a single protein encoded by a class I or class II HLA allele of a cell of the subject.

In some embodiments, the one or more proteins encoded by a class II HLA allele of a cell of the subject is two, three, four, five or six or more proteins encoded by a class I or class II HLA allele of a cell of the subject.

In some embodiments, the one or more proteins encoded by a class I or class II HLA allele of a cell of the subject is each protein encoded by a class I or class II HLA allele of a cell of the subject.

In some embodiments, the method further comprises administering to the subject a composition comprising one or more of the selected sub-set of peptide sequences.

In some embodiments, identifying the plurality of peptide sequences comprises comparing DNA, RNA, or protein sequences from cancer cells of the subject to DNA, RNA, or protein sequences from normal cells of the subject, wherein each of the plurality of the peptides comprise at least one mutation, which is present in the cancer cell of the subject, and not present in the normal cell of the subject.

In some embodiments, the machine-learning HLA-peptide presentation prediction model comprises a plurality of predictor variables identified at least based on the training data, wherein the training data comprises training peptide sequence information comprising amino acid position information, wherein the training peptide sequence information is associated with the HLA protein expressed in cells; and a function representing a relation between the amino acid position information and the presentation likelihood generated as output based on the amino acid position information and the plurality of predictor variables.

In some embodiments, identifying comprises identifying, based at least on the plurality of presentation predictions, a peptide sequence of the plurality of peptide sequences that has a probability greater than a threshold presentation prediction probability value of being presented by at least one of the one or more proteins encoded by a class I or class II HLA allele of a cell of the subject.

In some embodiments, one or more of the 0.2% of the plurality of test peptide sequences predicted to be presented by the machine learning HLA peptide presentation prediction model has a probability greater than the threshold presentation prediction probability value of being presented by at least one of the one or more proteins encoded by a class I or class II HLA allele of a cell of the subject.

In some embodiments, each of the 0.2% of the plurality of test peptide sequences predicted to be presented by the machine learning HLA peptide presentation prediction model has a probability greater than the threshold presentation prediction probability value of being presented by at least one of the one or more proteins encoded by a class I or class II HLA allele of a cell of the subject.

In some embodiments, the number of positives is constrained to be equal to the number of hits.

In some embodiments, the mass spectrometry is mono-allelic mass spectrometry.

In some embodiments, the peptides are presented by an HLA protein expressed in cells through autophagy.

In some embodiments, the peptides are presented by an HLA protein expressed in cells through phagocytosis.

In some embodiments, the plurality of predictor variables comprises expression level predictor of the source protein comprising the peptide.

In some embodiments, the plurality of predictor variables comprises stability predictor of the source protein comprising the peptide.

In some embodiments, the plurality of predictor variables comprises degradation rate predictor of the source protein comprising the peptide.

In some embodiments, the plurality of predictor variables comprises protein cleavability predictor of the source protein comprising the peptide.

In some embodiments, the plurality of predictor variables comprises cellular or tissue localization predictor of the source protein comprising the peptide.

In some embodiments, the plurality of predictor variables comprises a predictor for the intracellular processing mode of the source protein comprising the peptide, wherein processing mode of the source protein comprises predictor for whether the source protein is subject to autophagy, phagocytosis, and intracellular transport, among others.

In some embodiments, quality of the training data is increased by using a plurality of quality metrics.

In some embodiments, the plurality of quality metrics comprises common contaminant peptide removal, high scored peak intensity, high score, and high mass accuracy.

In some embodiments, a scored peak intensity is at least 50%.

In some embodiments, the scored peak intensity is at least 60%.

In some embodiments, a score is at least 7.

In some embodiments, a mass accuracy is at most 5 ppm.

In some embodiments, the peptides presented by an HLA protein expressed in cells are peptides presented by a single immunoprecipitated HLA protein expressed in cells.

In some embodiments, the peptides presented by an HLA protein expressed in cells are peptides presented by a single exogenous HLA protein expressed in cells.

In some embodiments, the peptides presented by an HLA protein expressed in cells are peptides presented by a single recombinant HLA protein expressed in cells.

In some embodiments, the plurality of predictor variables comprises a peptide-HLA affinity predictor variable.

In some embodiments, the peptides presented by the HLA protein comprise peptides identified by searching a no-enzyme specificity without modification peptide database.

In some embodiments, the peptides presented by the HLA protein comprise peptides identified by searching a peptide database using a reversed-database search strategy.

In some embodiments, the peptides presented by the HLA protein comprise peptides identified by comparing a MS/MS spectra of the HLA-peptides with MS/MS spectra of one or more peptides or proteins in a peptide or protein database.

In some embodiments, the mutation is selected from the group consisting of a point mutation, a splice site mutation, a frameshift mutation, a read-through mutation, and a gene fusion mutation.

In some embodiments, the peptides presented by the HLA protein have a length of from 8-12 or 15-40 amino acids.

In some embodiments, the peptides presented by the HLA protein comprise peptides identified by identifying peptides presented by an HLA protein by comparing a MS/MS spectra of the HLA-peptides with MS/MS spectra of one or more peptides or proteins in a peptide or protein database.

In some embodiments, the personalized cancer therapy further comprises an adjuvant.

In some embodiments, the personalized cancer therapy further comprises an immune checkpoint inhibitor.

In some embodiments, the training data comprises structured data, time-series data, unstructured data, relational data, or any combination thereof.

In some embodiments, the unstructured data comprises image data.

In some embodiments, the relational data comprises data from a customer system, an enterprise system, an operational system, a website, web accessible application program interface (API), or any combination thereof.

In some embodiments, the training data is uploaded to a cloud-based database.

In some embodiments, the training is performed using convolutional neural networks.

In some embodiments, the convolutional neural networks comprise at least two convolutional layers.

In some embodiments, the convolutional neural networks comprise at least one batch normalization step.

In some embodiments, the convolutional neural networks comprise at least one spatial dropout step.

In some embodiments, the convolutional neural networks comprise at least one global max pooling step.

In some embodiments, the convolutional neural networks comprise at least one dense layer.

In some embodiments, identifying peptide sequences comprises identifying peptide sequences with a mutation expressed in cancer cells of a subject.

In some embodiments, identifying peptide sequences comprises identifying peptide sequences not expressed in normal cells of a subject.

In some embodiments, identifying peptide sequences comprises identifying viral peptide sequences.

In some embodiments, identifying peptide sequences comprises identifying overexpressed peptide sequences.

Provided herein is a method for identifying HLA class I or class II specific peptides for immunotherapy for a subject, comprising: obtaining, by a computer processor, a candidate peptide comprising an epitope, and a plurality of peptide sequences, each comprising the epitope; processing, by a computer processor, amino acid information of the plurality of peptide sequences using a machine-learning HLA-peptide presentation prediction model to generate a presentation prediction for each of the plurality of peptide sequences to an immune cell, each presentation prediction indicative of a likelihood that one or more proteins encoded by an HLA class I or class II allele can present a given peptide sequence of the plurality of peptide sequences, wherein the machine-learning HLA-peptide presentation prediction model is trained using training data comprising sequence information of sequences of peptides presented by an HLA protein expressed in cells and identified by mass spectrometry; selecting a protein from the one or more proteins encoded by the HLA class I or class II allele of a cell of the subject, predicted to bind to the candidate peptide by the machine-learning HLA-peptide presentation prediction model, wherein the protein has a probability greater than a threshold presentation prediction probability value for presenting the candidate peptide to an immune cell; contacting the candidate peptide with the selected protein, such that the candidate peptide competes with a placeholder peptide associated with the selected protein; and identifying the candidate peptide as a peptide for immunotherapy specific for the selected protein based on whether the candidate peptide displaces the placeholder.

In some embodiments, obtaining comprises identifying the candidate peptide, wherein identifying the candidate peptide comprises comparing DNA, RNA, or protein sequences from cancer cells of the subject to DNA, RNA, or protein sequences from normal cells of the subject.

In some embodiments, processing comprises identifying a plurality of predictor variables based at least on the amino acid information of the plurality of peptide sequences, and processing the plurality of predictor variables using the machine-learning HLA-peptide presentation prediction model.

In some embodiments, the machine-learning HLA-peptide presentation prediction model comprises a plurality of predictor variables identified at least based on the training data, wherein the training data comprises: training peptide sequence information comprising amino acid position information, wherein the training peptide sequence information is associated with the HLA protein expressed in cells; and a function representing a relation between the amino acid position information and the presentation likelihood generated as output based on the amino acid position information and the plurality of predictor variables.

In some embodiments, the number of positives is constrained to be equal to the number of hits.

In some embodiments, the mass spectrometry is mono-allelic mass spectrometry.

In some embodiments, the plurality of predictor variables comprises any one or more of expression level predictor, stability predictor, degradation rate predictor, cleavability predictor, cellular or tissue localization predictor, and intracellular processing mode comprising autophagy, phagocytosis, and intracellular transport predictor, of the source protein comprising the peptide.

In some embodiments, quality of the training data is increased by using a plurality of quality metrics.

In some embodiments, the plurality of quality metrics comprises common contaminant peptide removal, high scored peak intensity, high score, and high mass accuracy.

In some embodiments, a scored peak intensity is at least 50%.

In some embodiments, the scored peak intensity is at least 60%.

In some embodiments, the placeholder peptide is a CLIP peptide.

In some embodiments, the placeholder peptide is a CMV peptide.

In some embodiments, the method further comprises measuring the IC₅₀ of displacement of the placeholder peptide by the target peptide.

In some embodiments, the IC₅₀ of displacement of the placeholder peptide by the target peptide is less than 500 nM.

In some embodiments, the target peptide is further identified by mass spectrometry.

In some embodiments, the at least one protein encoded by the HLA class I or class II allele of a cell of the subject is a recombinant protein.

In some embodiments, the at least one protein encoded by the HLA class I or class II allele of a cell of the subject is expressed in a eukaryotic cell.

In some embodiments, the peptides are presented by a HLA protein expressed in cells through autophagy.

In some embodiments, the peptides are presented by a HLA protein expressed in cells through phagocytosis.

In some embodiments, the peptides presented by a HLA protein expressed in cells are peptides presented by a single immunoprecipitated HLA protein expressed in cells.

In some embodiments, the peptides presented by a HLA protein expressed in cells are peptides presented by a single exogenous HLA protein expressed in cells.

In some embodiments, the peptides presented by a HLA protein expressed in cells are peptides presented by a single recombinant HLA protein expressed in cells.

In some embodiments, the plurality of predictor variables comprises a peptide-HLA affinity predictor variable.

In some embodiments, the peptides presented by the HLA protein comprise peptides identified by searching a no-enzyme specificity without modification peptide database.

In some embodiments, the peptides presented by the HLA protein comprise peptides identified by searching a peptide database using a reversed-database search strategy.

In some embodiments, the immunotherapy is cancer immunotherapy.

In some embodiments, the epitope is a cancer specific epitope.

In some embodiments, the identity of the peptide is known.

In some embodiments, the identity of the peptide is not known.

In some embodiments, the identity of the peptide is determined by mass spectrometry.

In some embodiments, peptide exchange assay comprises detection of peptide fluorescent probes or tags.

In some embodiments, in the placeholder peptide is a CLIP peptide. In some embodiments, the placeholder peptide has an amino acid sequence of PVSKMRMATPLLMQA (SEQ ID NO: 1).

In some embodiments, the polynucleic acid construct comprises an expression vector, further comprising one or more of: a promoter, a secretion signal, dimerization factors, ribosomal skipping sequence, one or more tags for purification and/or detection.

In some embodiments, the placeholder peptide sequence is encoded by a nucleic acid sequence within the vector.

In some embodiments, a sequence encoding a cleavable domain is placed in between the sequence encoding the placeholder peptide and the HLA beta1 peptide.

Provided herein is a method for assaying immunogenicity of a MHC class I or class II binding peptide, comprising: selecting a protein encoded by an HLA class I or class II allele predicted by a machine-learning HLA-peptide presentation prediction model to bind to the MHC class I or class II binding peptide, wherein the machine-learning HLA-peptide presentation prediction model is configured to generate a presentation prediction for a given peptide sequence, the presentation prediction indicative of a likelihood that one or more proteins encoded by the HLA class II allele can present the given peptide sequence, and wherein the protein has a probability greater than a threshold presentation prediction probability value for presenting the MHC class I or class II binding peptide; contacting the peptide with the selected protein such that the peptide competes with a placeholder peptide associated with the selected protein, and displaces the placeholder peptide, thereby forming a complex comprising the HLA class I or class II protein and the MHC class I or class II binding peptide; contacting the complex with a CD4+ T cell, and assaying for one or more of activation parameters of the CD4+ T cell, selected from the group consisting of: induction of a cytokine, induction of a chemokine, and expression of a cell surface marker.

Provided herein is a method for inducing a CD4+ T cell activation in a subject for cancer immunotherapy, the method comprising: identifying a peptide sequence associated with cancer and comprising a cancer mutation, wherein identifying the peptide sequence comprises comparing DNA, RNA, or protein sequences from cancer cells of the subject to DNA, RNA, or protein sequences from normal cells of the subject; selecting a protein encoded by an HLA class I or HLA class II allele that is normally expressed by a cell of the subject, and predicted by a machine-learning HLA-peptide presentation prediction model to bind to the peptide; wherein the prediction model has a positive predictive value of at least 0.1 at a recall rate of at least 0.1%, from 0.1%-50% or at most 50% and wherein the protein has a probability greater than a threshold presentation prediction probability value for presenting the identified peptide sequence; contacting the identified peptide with the selected protein encoded by the HLA class I or HLA class II allele to verify whether the identified peptide competes with a placeholder peptide associated with the selected protein encoded by the HLA class I or HLA class II allele to displace the placeholder peptide with an IC₅₀ value of less than 500 nM; optionally, purifying the identified peptide; and administering an effective amount of a polypeptide comprising a sequence of the identified peptide or a polynucleotide encoding the polypeptide to the subject.

Provided herein is a method of screening a drug comprising a polypeptide sequence for immunogenicity in a subject, comprising: obtaining, by a computer processor, a plurality of peptide sequences of the polypeptide sequence; processing, by a computer processor, amino acid information of the plurality of peptide sequences using a machine-learning HLA-peptide presentation prediction model to generate a presentation prediction for each of the plurality of peptide sequences, each presentation prediction indicative of a likelihood that one or more proteins encoded by a class I or II MHC allele of a cell of the subject can present an epitope sequence of a given peptide sequence of the plurality of peptide sequences, wherein the machine-learning HLA-peptide presentation prediction model is trained using training data comprising sequence information associated with the HLA protein expressed in cells; determining or predicting that each of the plurality of peptide sequences of the polypeptide sequence would not be immunogenic to the subject based on the plurality of presentation predictions; and administering to the subject a composition comprising the drug.

Provided herein is a method of screening a drug comprising a polypeptide sequence for immunogenicity in a subject, comprising: obtaining, by a computer processor, a plurality of peptide sequences of the polypeptide sequence; processing, by a computer processor, amino acid information of the plurality of peptide sequences using a machine-learning HLA-peptide presentation prediction model to generate a presentation prediction for each of the plurality of peptide sequences, each presentation prediction indicative of a likelihood that one or more proteins encoded by a class I or II MHC allele of a cell of the subject can present an epitope sequence of a given peptide sequence of the plurality of peptide sequences, wherein the machine-learning HLA-peptide presentation prediction model is trained using training data comprising sequence information of sequences of peptides presented by a HLA protein expressed in cells and identified by mass spectrometry; and determining or predicting that at least one of the plurality of peptide sequences of the polypeptide sequence would be immunogenic to the subject based on the plurality of presentation predictions.

Provided herein is a method of screening a drug comprising a polypeptide sequence for immunogenicity in a subject, the method comprising: inputting amino acid information of peptide sequences of the polypeptide sequence, using a computer processor, into a machine-learning HLA-peptide presentation prediction model to generate a set of presentation predictions for the peptide sequences, each presentation prediction representing a probability that one or more proteins encoded by a class I or II MHC allele of a cell of the subject will present an epitope sequence of a given peptide sequence; wherein the machine-learning HLA-peptide presentation prediction model comprises: a plurality of predictor variables identified at least based on training data, wherein the training data comprises: sequence information of sequences of peptides presented by a HLA protein expressed in cells and identified by mass spectrometry; training peptide sequence information comprising amino acid position information, wherein the training peptide sequence information is associated with the HLA protein expressed in cells; and a function representing a relation between the amino acid position information received as input and the presentation likelihood generated as output based on the amino acid position information and the predictor variables; determining or predicting that each of the peptide sequences of the polypeptide sequence would not be immunogenic to the subject based on the set of presentation predictions; and administering to the subject a composition comprising the drug.

Provided herein is a method of screening a drug comprising a polypeptide sequence for immunogenicity in a subject, the method comprising: inputting amino acid information of peptide sequences of the polypeptide sequence, using a computer processor, into a machine-learning HLA-peptide presentation prediction model to generate a set of presentation predictions for the peptide sequences, each presentation prediction representing a probability that one or more proteins encoded by a class I or II MHC allele of a cell of the subject will present an epitope sequence of a given peptide sequence; wherein the machine-learning HLA-peptide presentation prediction model comprises: a plurality of predictor variables identified at least based on training data; wherein the training data comprises: sequence information of sequences of peptides presented by a HLA protein expressed in cells and identified by mass spectrometry; training peptide sequence information comprising amino acid position information, wherein the training peptide sequence information is associated with the HLA protein expressed in cells; and a function representing a relation between the amino acid position information received as input and the presentation likelihood generated as output based on the amino acid position information and the predictor variables; determining or predicting that at least one of the peptide sequences of the polypeptide sequence would be immunogenic to the subject based on the set of presentation predictions.

Provided herein is a method of screening a drug comprising a polypeptide sequence for immunogenicity in a subject, comprising: obtaining, by a computer processor, a plurality of peptide sequences of the polypeptide sequence; processing, by a computer processor, amino acid information of the plurality of peptide sequences using a machine-learning HLA-peptide presentation prediction model to generate a presentation prediction for each of the plurality of peptide sequences, each presentation prediction indicative of a likelihood that one or more proteins encoded by a class I or II MHC allele of a cell of the subject can present an epitope sequence of a given peptide sequence of the plurality of peptide sequences, wherein the machine-learning HLA-peptide presentation prediction model is trained using training data comprising sequence information associated with the HLA protein expressed in cells; determining or predicting that each of the plurality of peptide sequences of the polypeptide sequence would not be immunogenic to the subject based on the plurality of presentation predictions; and administering to the subject a composition comprising the drug.

In some embodiments, the method further comprises deciding not to administer the drug to the subject.

In some embodiments, the drug comprises an antibody or binding fragment thereof.

In some embodiments, the peptide sequences of the polypeptide sequence have a length of 8, 9, 10, 11, or 12 amino acids, and wherein the protein encoded by a class I or II MHC allele of a cell of the subject is a protein encoded by a class I MHC allele of a cell of the subject.

In some embodiments, the peptide sequences of the polypeptide sequence have a length of 15, 16, 17, 18, 19, 20, 21, 22, 23, 24, or 25 amino acids, and wherein the protein encoded by a class I or II MHC allele of a cell of the subject is a protein encoded by a class II MHC allele of a cell of the subject.

Provided herein is a method of treating a subject with an autoimmune disease or condition comprising: (a) identifying or predicting an epitope of an expressed protein presented by a class I or II MHC of a cell of the subject, wherein a complex comprising the identified or predicted epitope and the class I or II MHC is targeted by a CD8 or CD4 T cell of the subject; (b) identifying a T cell receptor (TCR) that binds to the complex; (c) expressing the TCR in a regulatory T cell from the subject or an allogeneic regulatory T cell; and (d) administering the regulatory T cell expressing the TCR to the subject.

Provided herein is a method of treating a subject with an autoimmune disease or condition, comprising administering to the subject a regulatory T cell expressing a T cell receptor (TCR) that binds to a complex comprising: (i) an epitope of an expressed protein identified or predicted to be presented by a class I or II MHC of a cell of the subject, and (ii) the class I or II MHC, wherein the complex is targeted by a CD8 or CD4 T cell of the subject.

Provided herein is a computer system for identifying peptide sequences for a personalized cancer therapy of a subject, comprising: a database that is configured to store a plurality of peptide sequences of the subject; and one or more computer processors operatively coupled to said database, wherein said one or more computer processors are individually collectively programmed to: process amino acid information of the plurality of peptide sequences using a machine-learning HLA-peptide presentation prediction model to generate a presentation prediction for each of the plurality of peptide sequences, each presentation prediction indicative of a likelihood that one or more proteins encoded by a class I or class II MHC allele of a cell of the subject can present a given peptide sequence of the plurality of peptide sequences, wherein the machine-learning HLA-peptide presentation prediction model is trained using training data comprising sequence information of sequences of peptides presented by an HLA protein expressed in cells and identified by mass spectrometry; and select a subset of the plurality of peptide sequences for the personalized cancer therapy of the subject based at least on the plurality of presentation predictions.

Provided herein is a computer system for identifying HLA class I or HLA class II specific peptides for immunotherapy for a subject, comprising: a database that is configured to store a candidate peptide comprising an epitope, and a plurality of peptide sequences, each comprising the epitope; and one or more computer processors operatively coupled to said database, wherein said one or more computer processors are individually collectively programmed to: process amino acid information of the plurality of peptide sequences a machine-learning HLA-peptide presentation prediction model to generate a presentation prediction for each of the plurality of peptide sequences to an immune cell, each presentation prediction indicative of a likelihood that one or more proteins encoded by an HLA class I or HLA class II allele can present a given peptide sequence of the plurality of peptide sequences, wherein the machine-learning HLA-peptide presentation prediction model is trained using training data comprising sequence information of sequences of peptides presented by an HLA protein expressed in cells and identified by mass spectrometry; select a protein from the one or more proteins encoded by the HLA class I or HLA class II allele of a cell of the subject, predicted to bind to the candidate peptide by the machine-learning HLA-peptide presentation prediction model, wherein the protein has a probability greater than a threshold presentation prediction probability value for presenting the candidate peptide to an immune cell; and identify the candidate peptide as a peptide for immunotherapy specific for the selected protein based on whether the candidate peptide displaces the placeholder peptide, upon contacting the candidate peptide with the selected protein, such that the candidate peptide competes with a placeholder peptide associated with the selected protein.

Provided herein is a computer system for screening a drug comprising a polypeptide sequence for immunogenicity in a subject, comprising: a database that is configured to store a plurality of peptide sequences of the polypeptide sequence; and one or more computer processors operatively coupled to said database, wherein said one or more computer processors are individually collectively programmed to: process amino acid information of the plurality of peptide sequences using a machine-learning HLA-peptide presentation prediction model to generate a presentation prediction for each of the plurality of peptide sequences, each presentation prediction indicative of a likelihood that one or more proteins encoded by a class I or II MHC allele of a cell of the subject can present an epitope sequence of a given peptide sequence of the plurality of peptide sequences, wherein the machine-learning HLA-peptide presentation prediction model is trained using training data comprising sequence information associated with the HLA protein expressed in cells; and determine or predict that each of the plurality of peptide sequences of the polypeptide sequence would not be immunogenic to the subject based on the plurality of presentation predictions, wherein a composition comprising the drug is administered to the subject.

Provided herein is a computer system for screening a drug comprising a polypeptide sequence for immunogenicity in a subject, comprising: a database that is configured to store a plurality of peptide sequences of the polypeptide sequence; and one or more computer processors operatively coupled to said database, wherein said one or more computer processors are individually collectively programmed to: process amino acid information of the plurality of peptide sequences using a machine-learning HLA-peptide presentation prediction model to generate a presentation prediction for each of the plurality of peptide sequences, each presentation prediction indicative of a likelihood that one or more proteins encoded by a class I or II MHC allele of a cell of the subject can present an epitope sequence of a given peptide sequence of the plurality of peptide sequences, wherein the machine-learning HLA-peptide presentation prediction model is trained using training data comprising sequence information of sequences of peptides presented by a HLA protein expressed in cells and identified by mass spectrometry; and determine or predict that at least one of the plurality of peptide sequences of the polypeptide sequence would be immunogenic to the subject based on the plurality of presentation predictions.

Provided herein is a non-transitory computer readable medium comprising machine-executable code that, upon execution by one or more computer processors, implements a method for identifying peptide sequences for a personalized cancer therapy of a subject, said method comprising: obtaining a plurality of peptide sequences of the subject; processing amino acid information of the plurality of peptide sequences using a machine-learning HLA-peptide presentation prediction model to generate a presentation prediction for each of the plurality of peptide sequences, each presentation prediction indicative of a likelihood that one or more proteins encoded by a class I or class II MHC allele of a cell of the subject can present a given peptide sequence of the plurality of peptide sequences, wherein the machine-learning HLA-peptide presentation prediction model is trained using training data comprising sequence information of sequences of peptides presented by an HLA protein expressed in cells and identified by mass spectrometry; and selecting a subset of the plurality of peptide sequences for the personalized cancer therapy of the subject based at least on the plurality of presentation predictions.

Provided herein is a non-transitory computer readable medium comprising machine-executable code that, upon execution by one or more computer processors, implements a method for identifying HLA class II specific peptides for immunotherapy for a subject, comprising: obtaining a candidate peptide comprising an epitope, and a plurality of peptide sequences, each comprising the epitope; processing amino acid information of the plurality of peptide sequences a machine-learning HLA-peptide presentation prediction model to generate a presentation prediction for each of the plurality of peptide sequences to an immune cell, each presentation prediction indicative of a likelihood that one or more proteins encoded by an HLA class I or HLA class II allele can present a given peptide sequence of the plurality of peptide sequences, wherein the machine-learning HLA-peptide presentation prediction model is trained using training data comprising sequence information of sequences of peptides presented by an HLA protein expressed in cells and identified by mass spectrometry; selecting a protein from the one or more proteins encoded by the HLA class II allele of a cell of the subject, predicted to bind to the candidate peptide by the machine-learning HLA-peptide presentation prediction model, wherein the protein has a probability greater than a threshold presentation prediction probability value for presenting the candidate peptide to an immune cell; and identifying the candidate peptide as a peptide for immunotherapy specific for the selected protein based on whether the candidate peptide displaces the placeholder peptide, upon contacting the candidate peptide with the selected protein, such that the candidate peptide competes with a placeholder peptide.

Provided herein is a non-transitory computer readable medium comprising machine-executable code that, upon execution by one or more computer processors, implements a method of screening a drug comprising a polypeptide sequence for immunogenicity in a subject, comprising: obtaining a plurality of peptide sequences of the polypeptide sequence; processing amino acid information of the plurality of peptide sequences using a machine-learning HLA-peptide presentation prediction model to generate a presentation prediction for each of the plurality of peptide sequences, each presentation prediction indicative of a likelihood that one or more proteins encoded by a class I or II MHC allele of a cell of the subject can present an epitope sequence of a given peptide sequence of the plurality of peptide sequences, wherein the machine-learning HLA-peptide presentation prediction model is trained using training data comprising sequence information associated with the HLA protein expressed in cells; and determining or predicting that each of the plurality of peptide sequences of the polypeptide sequence would not be immunogenic to the subject based on the plurality of presentation predictions, wherein a composition comprising the drug is administered to the subject.

Provided herein is a non-transitory computer readable medium comprising machine-executable code that, upon execution by one or more computer processors, implements a method of screening a drug comprising a polypeptide sequence for immunogenicity in a subject, comprising: obtaining a plurality of peptide sequences of the polypeptide sequence; processing amino acid information of the plurality of peptide sequences using a machine-learning HLA-peptide presentation prediction model to generate a presentation prediction for each of the plurality of peptide sequences, each presentation prediction indicative of a likelihood that one or more proteins encoded by a class I or II MHC allele of a cell of the subject can present an epitope sequence of a given peptide sequence of the plurality of peptide sequences, wherein the machine-learning HLA-peptide presentation prediction model is trained using training data comprising sequence information of sequences of peptides presented by a HLA protein expressed in cells and identified by mass spectrometry; and determining or predicting that at least one of the plurality of peptide sequences of the polypeptide sequence would be immunogenic to the subject based on the plurality of presentation predictions.

Provided herein is a method comprising: processing amino acid information of a plurality of candidate peptide sequences using a machine learning HLA peptide presentation prediction model to generate a plurality of presentation predictions, wherein each candidate peptide sequences of the plurality is encoded by a genome or exome of a subject, wherein the plurality of presentation predictions comprises an HLA presentation prediction for each of the plurality of candidate peptide sequences, wherein each presentation prediction indicative of a likelihood that one or more proteins encoded by a HLA class I or HLA class II allele of a cell of the subject can present a given candidate peptide sequence of the plurality, wherein the machine learning HLA peptide presentation prediction model is trained using training data comprising sequence information of sequences of training peptides identified by mass spectrometry to be presented by an HLA protein expressed in training cells; and identifying, based at least on the plurality of presentation predictions, a peptide sequence of the plurality of peptide sequences that has a probability greater than a threshold presentation prediction probability value of being presented by at least one of the one or more proteins encoded by a HLA class I or HLA class II allele of a cell of the subject; wherein the machine learning HLA peptide presentation prediction model has a positive predictive value (PPV) of at least 0.07 when amino acid information of a plurality of test peptide sequences are processed to generate a plurality of test presentation predictions, each test presentation prediction indicative of a likelihood that the one or more proteins encoded by a HLA class I or HLA class II allele of a cell of the subject can present a given test peptide sequence of the plurality of test peptide sequences, wherein the plurality of test peptide sequences comprises at least 500 test peptide sequences comprising (i) at least one hit peptide sequence identified by mass spectrometry to be presented by an HLA protein expressed in cells and (ii) at least 499 decoy peptide sequences contained within a protein encoded by a genome of an organism, wherein the organism and the subject are the same species, wherein the plurality of test peptide sequences comprises a ratio of 1:499 of the at least one hit peptide sequence to the at least 499 decoy peptide sequences and 0.2% of the plurality of test peptide sequences are predicted to be presented by the HLA protein expressed in cells by the machine learning HLA peptide presentation prediction model.

Provided herein is a method comprising: processing amino acid information of a plurality of peptide sequences of encoded by a genome or exome of a subject using a machine-learning HLA-peptide binding prediction model to generate a plurality of binding predictions, wherein the plurality of binding predictions comprises an HLA binding prediction for each of the plurality of candidate peptide sequences, each binding prediction indicative of a likelihood that one or more proteins encoded by a HLA class I or HLA class II of a cell of the subject binds to a given candidate peptide sequence of the plurality of candidate peptide sequences, wherein the machine learning HLA peptide binding prediction model is trained using training data comprising sequence information of sequences of peptides identified to bind to an HLA class I or HLA class II protein or an HLA class I or HLA class II protein analog; and identifying, based at least on the plurality of binding predictions, a peptide sequence of the plurality of peptide sequences that has a probability greater than a threshold binding prediction probability value of binding to at least one of the one or more proteins encoded by a HLA class I or HLA class II allele of a cell of the subject; wherein the machine learning HLA peptide binding prediction model has a positive predictive value (PPV) of at least 0.1 when amino acid information of a plurality of test peptide sequences are processed to generate a plurality of test binding predictions, each test binding prediction indicative of a likelihood that the one or more proteins encoded by a HLA class I or HLA class II of a cell of the subject binds to a given test peptide sequence of the plurality of test peptide sequences, wherein the plurality of test peptide sequences comprises at least 50 test peptide sequences comprising (i) at least one hit peptide sequence identified by mass spectrometry to be presented by an HLA protein expressed in cells and (ii) at least 19 decoy peptide sequences contained within a protein comprising a peptide sequence identified by mass spectrometry to be presented by an HLA protein expressed in cells, wherein the organism and the subject are the same species, wherein the plurality of test peptide sequences comprises a ratio of 1:19 of the at least one hit peptide sequence to the at least 19 decoy peptide sequences and 5% of the plurality of test peptide sequences are predicted to bind to the HLA protein expressed in cells by the machine learning HLA peptide presentation prediction model.

In some embodiments, the machine learning HLA peptide presentation prediction model is trained using training data comprising sequence information of sequences of training peptides identified by mass spectrometry to be presented by an HLA protein expressed in training cells.

In some embodiments, one or more of the 0.2% of the plurality of test peptide sequences predicted to be presented by the by the machine learning HLA peptide presentation prediction model has a probability greater than the threshold presentation prediction probability value of being presented by at least one of the one or more proteins encoded by a HLA class I or HLA class II allele of a cell of the subject.

In some embodiments, each of the 0.2% of the plurality of test peptide sequences predicted to be presented by the by the machine learning HLA peptide presentation prediction model has a probability greater than the threshold presentation prediction probability value of being presented by at least one of the one or more proteins encoded by a HLA class I or HLA class II allele of a cell of the subject.

Provided herein is a method for preparing a personalized cancer therapy, the method comprising: identifying peptide sequences, wherein the peptide sequences are associated with cancer, wherein identifying comprises comparing DNA, RNA or protein sequences from the cancer cells of the subject to DNA, RNA or protein sequences from the normal cells of the subject; inputting amino acid position information of the peptide sequences identified, using a computer processor, into a machine-learning HLA-peptide presentation prediction model to generate a set of presentation predictions for the peptide sequences identified, each presentation prediction representing a probability that one or more proteins encoded by an HLA class I or HLA class II allele of a cell of the subject will present a given sequence of a peptide sequence identified; wherein the machine-learning HLA-peptide presentation prediction model comprises: a plurality of predictor variables identified at least based on training data wherein the training data comprises: sequence information of sequences of peptides presented by an HLA protein expressed in cells and identified by mass spectrometry; training peptide sequence information comprising amino acid position information, wherein the training peptide sequence information is associated with the HLA protein expressed in cells; and a function representing a relation between the amino acid position information received as input and the presentation likelihood generated as output based on the amino acid position information and the predictor variables; and selecting a subset of the peptide sequences identified based on the set of presentation predictions for preparing the personalized cancer therapy; wherein the prediction model has a positive predictive value of at least 0.1 at a recall rate of at least 0.1%, from 0.1%-50% or at the most 50%.

Provided herein is a method comprising training a machine-learning HLA-peptide presentation prediction model, wherein training comprises inputting amino acid position information sequences of HLA-peptides isolated from one or more HLA-peptide complexes from a cell expressing an HLA class II allele into the HLA-peptide presentation prediction model using a computer processor; the machine-learning HLA-peptide presentation prediction model comprising: a plurality of predictor variables identified at least based on training data that comprises: sequence information of sequences of peptides presented by an HLA protein expressed in cells and identified by mass spectrometry; training peptide sequence information comprising amino acid position information of training peptides, wherein the training peptide sequence information is associated with the HLA protein expressed in cells; and a function representing a relation between the amino acid position information received as input and a presentation likelihood generated as output based on the amino acid position information and the predictor variables.

In some embodiments, the presentation model has a positive predictive value of at least 0.25 at a recall rate at least 0.1%, from 0.1%-50% or at the most 50%.

In some embodiments, the presentation model has a positive predictive value of at least 0.4 at a recall rate of at least 0.1%, from 0.1%-50% or at the most 50%.

In some embodiments, the presentation model has a positive predictive value of at least 0.6 at a recall rate of at least 0.1%, from 0.1%-50% or at the most 50%.

In some embodiments, the mass spectrometry is mono-allelic mass spectrometry.

In some embodiments, the peptides are presented by an HLA protein expressed in cells through autophagy.

In some embodiments, the peptides are presented by an HLA protein expressed in cells through phagocytosis.

In some embodiments, quality of the training data is increased by using a plurality of quality metrics.

In some embodiments, the plurality of quality metrics comprises common contaminant peptide removal, high scored peak intensity, high score, and high mass accuracy.

In some embodiments, the scored peak intensity is at least 50%.

In some embodiments, the scored peak intensity is at least 60%.

In some embodiments, a score is at least 7.

In some embodiments, a mass accuracy is at most 5 ppm.

In some embodiments, a mass accuracy is at most 2 ppm.

In some embodiments, a backbone cleavage score is at least 5.

In some embodiments, a backbone cleavage score is at least 8.

In some embodiments, the peptides presented by an HLA protein expressed in cells are peptides presented by a single immunoprecipitated HLA protein expressed in cells.

In some embodiments, the peptides presented by an HLA protein expressed in cells are peptides presented by a single exogenous HLA protein expressed in cells.

In some embodiments, the peptides presented by an HLA protein expressed in cells are peptides presented by a single recombinant HLA protein expressed in cells.

In some embodiments, the plurality of predictor variables comprises a peptide-HLA affinity predictor variable.

In some embodiments, the plurality of predictor variables comprises a source protein expression level predictor variable.

In some embodiments, the plurality of predictor variables comprises a peptide cleavability predictor variable.

In some embodiments, the training peptide sequence information comprises sequences from the peptides presented by the HLA protein, which comprise peptides identified by searching a no-enzyme specificity without modification to a peptide database. In some embodiments, the peptides presented by the HLA protein comprise peptides identified by searching the de novo peptide sequencing tools.

In some embodiments, the peptides presented by the HLA protein comprise peptides identified by searching a peptide database using a reversed-database search strategy.

In some embodiments, the HLA protein comprises an HLA-DR, and HLA-DP or an HLA-DQ protein. In some embodiments, the HLA protein comprises an HLA-DR protein selected from the group consisting of an HLA-DR, and HLA-DP or an HLA-DQ protein. In some embodiments, the HLA protein comprises an HLA-DR protein selected from the group consisting of HLA-DPB1*01:01/HLA-DPA1*01:03, HLA-DPB1*02:01/HLA-DPA1*01:03, HLA-DPB1*03:01/HLA-DPA1*01:03, HLA-DPB1*04:01/HLA-DPA1*01:03, HLA-DPB1*04:02/HLA-DPA1*01:03, HLA-DPB1*06:01/HLA-DPA1*01:03, HLA-DQB1*02:01/HLA-DQA1*05:01, HLA-DQB1*02:02/HLA-DQA1*02:01, HLA-DQB1*06:02/HLA-DQA1*01:02, HLA-DQB1*06:04/HLA-DQA1*01:02, HLA-DRB1*01:01, HLA-DRB1*01:02, HLA-DRB1*03:01, HLA-DRB1*03:02, HLA-DRB1*04:01, HLA-DRB1*04:02, HLA-DRB1*04:03, HLA-DRB1*04:04, HLA-DRB1*04:05, HLA-DRB1*04:07, HLA-DRB1*07:01, HLA-DRB1*08:01, HLA-DRB1*08:02, HLA-DRB1*08:03, HLA-DRB1*08:04, HLA-DRB1*09:01, HLA-DRB1*10:01, HLA-DRB1*11:01, HLA-DRB1*11:02, HLA-DRB1*11:04, HLA-DRB1*12:01, HLA-DRB1*12:02, HLA-DRB1*13:01, HLA-DRB1*13:02, HLA-DRB1*13:03, HLA-DRB1*14:01, HLA-DRB1*15:01, HLA-DRB1*15:02, HLA-DRB1*15:03, HLA-DRB1*16:01, HLA-DRB3*01:01, HLA-DRB3*02:02, HLA-DRB3*03:01, HLA-DRB4*01:01, and HLA-DRB5*01:01.

In some embodiments, the peptides presented by the HLA protein comprise peptides identified by comparing MS/MS spectra of the HLA-peptides with MS/MS spectra of one or more HLA-peptides in a peptide database.

In some embodiments, the mutation is selected from the group consisting of a point mutation, a splice site mutation, a frameshift mutation, a read-through mutation, and a gene fusion mutation.

In some embodiments, the peptides presented by the HLA protein have a length of 8-12 or 15-40 amino acids.

In some embodiments, the peptides presented by the HLA protein comprise peptides identified by (a) isolating one or more HLA complexes from a cell line expressing a single HLA class I or HLA class II allele; (b) isolating one or more HLA-peptides from the one or more isolated HLA complexes; (c) obtaining MS/MS spectra for the one or more isolated HLA-peptides; and (d) obtaining a peptide sequence that corresponds to the MS/MS spectra of the one or more isolated HLA-peptides from a peptide database; wherein one or more sequences obtained from step (d) identifies the sequence of the one or more isolated HLA-peptides.

In some embodiments, the personalized cancer therapy further comprises an adjuvant.

In some embodiments, the personalized cancer therapy further comprises an immune checkpoint inhibitor.

In some embodiments, the training data comprises structured data, time-series data, unstructured data, relational data, or any combination thereof.

In some embodiments, the unstructured data comprises image data.

In some embodiments, the relational data comprises data from a customer system, an enterprise system, an operational system, a website, web accessible application program interface (API), or any combination thereof.

In some embodiments, the training data is uploaded to a cloud-based database.

In some embodiments, the training is performed using convolutional neural networks.

In some embodiments, the convolutional neural networks comprise at least two convolutional layers.

In some embodiments, the convolutional neural networks (CNN) comprise at least one batch normalization step.

In some embodiments, the convolutional neural networks comprise at least one spatial dropout step.

In some embodiments, the convolutional neural networks comprise at least one global max pooling step.

In some embodiments, the convolutional neural networks comprise at least one dense layer.

In some embodiments, identifying peptide sequences comprises identifying peptide sequences with a mutation expressed in cancer cells of a subject.

In some embodiments, identifying peptide sequences comprises identifying peptide sequences not expressed in normal cells of a subject.

In some embodiments, identifying peptide sequences comprises identifying overexpressed peptide sequences.

In some embodiments, identifying peptide sequences comprises identifying viral peptide sequences. In one aspect, provided herein is a method for identifying HLA class I or HLA class II specific peptides for immunotherapy specific for a subject, the method comprising: identifying a candidate peptide comprising an epitope; inputting amino acid information of a plurality of peptide sequences, each comprising an epitope, using a computer processor, into a machine-learning HLA-peptide presentation prediction model to generate a set of HLA presentation predictions for the peptide sequence to an immune cell, each presentation prediction representing a probability that one or more proteins encoded by an HLA class I or HLA class II allele of a cell of the subject will present a given peptide sequence comprising the epitope; wherein the prediction model has a positive predictive value of at least 0.1 at a recall rate of at least 0.10%, from 0.1%-50% or at the most 50%, selecting a protein from the one or more proteins encoded by the HLA class I or HLA class II allele of a cell of the subject, predicted to bind to the candidate peptide by the prediction model, wherein the protein has a probability greater than a threshold presentation prediction probability value for presenting the candidate peptide to an immune cell; contacting the candidate peptide with the protein encoded by the HLA class I or HLA class II allele, such that the candidate peptide competes with a placeholder peptide associated with the protein encoded by the HLA class I or HLA class II allele; and, identifying the candidate peptide as a peptide for immunotherapy specific for the protein encoded by an HLA class II allele based on whether the candidate peptide displaces the placeholder peptide.

In some embodiments, the immunotherapy is cancer immunotherapy.

In some embodiments, identifying comprises comparing DNA, RNA or protein sequences from the cancer cells of the subject to DNA, RNA or protein sequences from the normal cells of the subject. In some embodiments, the epitope is a cancer specific epitope.

In some embodiments, the placeholder peptide is a CLIP peptide. In some embodiments, the placeholder peptide is a CMV peptide. In some embodiments, the method further comprises measuring the IC₅₀ of displacement of the placeholder peptide by the target peptide. In some embodiments, the IC₅₀ of displacement of the placeholder peptide by the target peptide is less than 500 nM. In some embodiments, the target peptide is further identified by mass spectrometry. In some embodiments, the at least one protein encoded by the HLA class I or HLA class II allele of a cell of the subject is a recombinant protein. In some embodiments, the at least one protein encoded by the HLA class I or HLA class II allele of a cell of the subject is expressed in a eukaryotic cell.

In one aspect, provided herein is assay method for verifying the specificity of a candidate peptide for binding an HLA class I or HLA class II protein, the method comprising: expressing in a eukaryotic cell, a polynucleic acid construct comprising a nucleic acid sequence encoding an HLA class I or HLA class II protein comprising an alpha chain and beta chain or portions thereof, capable of binding a peptide comprising an MHC-binding epitope, and wherein the expressed HLA class I or HLA class II protein or portions thereof remains associated with a placeholder peptide; isolating the HLA class I or HLA class II protein or portions thereof expressed in the eukaryotic cell; performing a peptide exchange assay by (a) adding increasing amount of the candidate peptide to determine whether the candidate peptide displaces the placeholder peptide associated with the HLA class I or HLA class II protein or portions thereof; and (b) calculating the IC₅₀ of the displacement reaction to determine the affinity of the candidate peptide to the HLA class I or HLA class II protein or portions thereof relative to the placeholder peptide, thereby verifying the specificity of the candidate peptide for binding an HLA class I or HLA class II protein.

In some embodiments, the identity of the peptide is known. In some embodiments, the identity of the peptide is not known. In some embodiments, the identity of the peptide is determined by mass spectrometry.

In some embodiments, the peptide exchange assay comprises detection of peptide fluorescent probes or tags. In some embodiments, the placeholder peptide is a CLIP peptide.

In some embodiments, the polynucleic acid construct comprises an expression vector, further comprising one or more of: a promoter, a linker, one or more protease cleavage sites, a secretion signal, dimerization factors, ribosomal skipping sequence, one or more tags for purification and or detection.

In one aspect, provided herein is a method for assaying immunogenicity of a MHC class II binding peptide, the method comprising: selecting a protein encoded by an HLA class II allele predicted by a machine-learning HLA-peptide presentation prediction model to bind to the peptide; wherein the prediction model has a positive predictive value of at least 0.1 at a recall rate of at least 0.1%, from 0.1%-50% or at the most 50% and wherein the protein has a probability greater than a threshold presentation prediction probability value for presenting the identified peptide sequence; contacting the peptide with the selected protein encoded by the HLA class II allele such that the peptide competes with a placeholder peptide associated with the selected protein encoded by the HLA class II allele, and displaces the placeholder peptide, thereby forming a complex comprising the HLA class II protein and the identified peptide; contacting the HLA class II protein and the identified peptide complex with a CD4+ T cell, assaying for one or more of activation parameters of the CD4+ T cell, selected from induction of a cytokine, induction of a chemokine and expression of a cell surface marker.

In one aspect, provided herein is a method of screening a drug comprising a polypeptide sequence for immunogenicity in a subject, the method comprising: inputting amino acid information of peptide sequences of the polypeptide sequence, using a computer processor, into a machine-learning HLA-peptide presentation prediction model to generate a set of presentation predictions for the peptide sequences, each presentation prediction representing a probability that one or more proteins encoded by an HLA class I or II allele of a cell of the subject will present an epitope sequence of a given peptide sequence; wherein the machine-learning HLA-peptide presentation prediction model comprises: a plurality of predictor variables identified at least based on training data wherein the training data comprises: sequence information of sequences of peptides presented by an HLA protein expressed in cells and identified by mass spectrometry; training peptide sequence information comprising amino acid position information, wherein the training peptide sequence information is associated with the HLA protein expressed in cells; and a function representing a relation between the amino acid position information received as input and the presentation likelihood generated as output based on the amino acid position information and the predictor variables; (b) determining or predicting that each of the peptide sequences of the polypeptide sequence would not be immunogenic to the subject based on the set of presentation predictions; and (c) administering to the subject a composition comprising the drug.

In one aspect, provided herein is a method of screening a drug comprising a polypeptide sequence for immunogenicity in a subject, the method comprising: (a) inputting amino acid information of peptide sequences of the polypeptide sequence, using a computer processor, into a machine-learning HLA-peptide presentation prediction model to generate a set of presentation predictions for the peptide sequences, each presentation prediction representing a probability that one or more proteins encoded by an HLA class I or II allele of a cell of the subject will present an epitope sequence of a given peptide sequence; wherein the machine-learning HLA-peptide presentation prediction model comprises: a plurality of predictor variables identified at least based on training data; wherein the training data comprises: sequence information of sequences of peptides presented by an HLA protein expressed in cells and identified by mass spectrometry; training peptide sequence information comprising amino acid position information, wherein the training peptide sequence information is associated with the HLA protein expressed in cells; and a function representing a relation between the amino acid position information received as input and the presentation likelihood generated as output based on the amino acid position information and the predictor variables; (b) determining or predicting that at least one of the peptide sequences of the polypeptide sequence would be immunogenic to the subject based on the set of presentation predictions.

In one embodiment, the method further comprises deciding not to administer the drug to the subject.

In one embodiment, the drug comprises an antibody or binding fragment thereof.

In one embodiment, the peptide sequences of the polypeptide sequences comprise each contiguous peptide sequence of the polypeptide sequence that has a length of 8, 9, 10, 11 or 12 amino acids, and wherein the protein encoded by an HLA class I or II allele of a cell of the subject is a protein encoded by an HLA class I allele of a cell of the subject.

In one embodiment, the peptide sequences of the polypeptide sequences comprise each contiguous peptide sequence of the polypeptide sequence that has a length of 15, 16, 17, 18, 19, 20, 21, 22, 23, 24, or 25 amino acids, and wherein the protein encoded by an HLA class I or II allele of a cell of the subject is a protein encoded by a class II MHC allele of a cell of the subject.

In one aspect, provided herein is a method of treating a subject with an autoimmune disease or condition comprising: (a) identifying or predicting an epitope of an expressed protein presented by an HLA class I or II of a cell of the subject, wherein a complex comprising the identified or predicted epitope and the HLA class I or II is targeted by a CD8 or CD4 T cell of the subject; (b) identifying a T cell receptor (TCR) that binds to the complex; (c) expressing the TCR in a regulatory T cell from the subject or an allogeneic regulatory T cell; and (d) administering the regulatory T cell expressing the TCR to the subject.

In one embodiment, the autoimmune disease or condition is diabetes.

In one embodiment, the cell is an islet cell.

In one aspect, provided herein is a method of treating a subject with an autoimmune disease or condition comprising administering to the subject a regulatory T cell expressing a T cell receptor (TCR) that binds to a complex comprising (i) an epitope of an expressed protein identified or predicted to be presented by an HLA class I or II of a cell of the subject and (ii) the HLA class I or II, wherein the complex is targeted by a CD8 or CD4 T cell of the subject.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

In one aspect, provided herein is a method for treating a cancer in a subject the method comprising: identifying peptide sequences, wherein the peptide sequences are associated with cancer, wherein identifying comprises comparing DNA, RNA or protein sequences from the cancer cells of the subject to DNA, RNA or protein sequences from the normal cells of the subject; inputting amino acid information of the peptide sequences identified, using a computer processor, into a machine-learning HLA-peptide presentation prediction model to generate a set of presentation predictions for the peptide sequences identified, each presentation prediction representing a probability that one or more proteins encoded by an HLA class I or HLA class II allele of a cell of the subject will present a given sequence of a peptide sequence identified; wherein the machine-learning HLA-peptide presentation prediction model comprises: a plurality of predictor variables identified at least based on training data wherein the training data comprises: sequence information of sequences of peptides presented by an HLA protein expressed in cells and identified by mass spectrometry; training peptide sequence information comprising amino acid position information, wherein the training peptide sequence information is associated with the HLA protein expressed in cells; and a function representing a relation between the amino acid position information received as input and the presentation likelihood generated as output based on the amino acid position information and the predictor variables; and selecting a subset of the peptide sequences identified based on the set of presentation predictions for preparing the personalized cancer therapy; and administering to the subject a composition comprising one or more of the peptides, wherein the prediction model has a positive predictive value of at least 0.1 at a recall rate of at least 0.1%, from 0.1%-50% or at most 50%.

In some embodiments, the machine-learning HLA-peptide presentation prediction model comprises sequence information of sequences of peptides presented by an HLA protein expressed in cells and identified by mass spectrometry after performing reverse phase offline fractionation.

In some embodiments, the prediction model exhibits a 1.1× to 100× fold improvement compared to NetMHCIIpan or NetMHCI. In some embodiments, the prediction model exhibits a 1.1, 2, 3, 4, 5, 6, 7, 7.4, 8, 9, 10, 11, 12, 13, 14, 15, 16, 17, 18, 18, 20, 21, 22, 23, 24, 25, 26, 27, 28, 29, 30, 31, 32, 33, 34, 35, 36, 37, 38, 39, 40, 41, 42, 43, 44, 45, 50, 55, 60, 61, 62, 63, 64, 65, 66, 67, 68, 69, 70, 71, 72, 73, 74, 75, 76, 77, 78, 79, 80, 81, 8, 83, 84, 85, 86, 87, 88, 89, 90, 91, 92, 93, 94, 95, 96, 97, 98, 99, 100-fold or more improvement compared to NetMHCIIpan or NetMHCI.

In one aspect, the present disclosure provides method for predicting peptides that can accurately pair with, or bind to, a specific HLA class I or class II molecule, such that the high fidelity binding of the peptide to HLA class I or class II protein (comprising the alpha and beta chain heterodimer) ensures presentation of the specific peptide to the T lymphocytes, thereby eliciting a specific immune response and avoid any cross-reactivity or immune promiscuity. Several recent studies have shown that CD8+ or CD4+ T cells can also recognize HLA class I or class II presented ligands and contribute to tumor control. Cancer vaccines and other immunotherapies would ideally take advantage of directing CD8+ or CD4+ T cell responses, but current efforts have forgone HLA class I or class II antigen prediction entirely because the accuracy of current prediction tools is inadequate.

In one aspect, the present disclosure provides method for predicting peptides that can accurately bind to a specific HLA class I or class II protein, such that a more sustained and robust immune response can be activated with the peptide, when the peptide is administered therapeutically to a subject expressing the specific cognate HLA class I or class II protein, by means of the ability of HLA class I or class II protein's activation of CD8+ or CD4+ T cells and stimulate immunological memory. In some embodiments, the method provided herein exhibits an improvement in a specific HLA class I or class II protein prediction over currently available predictor. In some embodiments, the method provided herein exhibits at least about a 1.1-fold improvement in a specific HLA class I or class II protein prediction over currently available predictor. In some embodiments, the method provided herein exhibits at least about a 2-fold improvement in a specific HLA class I or class II protein prediction over currently available predictor. In some embodiments, the method provided herein exhibits at least about a 3-fold improvement in a specific HLA class I or class II protein prediction over currently available predictor. In some embodiments, the method provided herein exhibits at least about a 4-fold improvement in a specific HLA class I or class II protein prediction over currently available predictor. In some embodiments, the method provided herein exhibits at least about a 5-fold improvement in a specific HLA class I or class II protein prediction over currently available predictor. In some embodiments, the method provided herein exhibits at least about a 6-fold improvement in a specific HLA class I or class II protein prediction over currently available predictor. In some embodiments, the method provided herein exhibits at least about a 7-fold improvement in a specific HLA class I or class II protein prediction over currently available predictor. In some embodiments, the method provided herein exhibits at least about a 8-fold improvement in a specific HLA class I or class II protein prediction over currently available predictor. In some embodiments, the method provided herein exhibits at least about a 9-fold improvement in a specific HLA class I or class II protein prediction over currently available predictor. In some embodiments, the method provided herein exhibits at least about a 10-fold improvement in a specific HLA class I or class II protein prediction over currently available predictor. In some embodiments, the method provided herein exhibits at least about a 15-fold improvement in a specific HLA class I or class II protein prediction over currently available predictor. In some embodiments, the method provided herein exhibits at least about a 20-fold improvement in a specific HLA class I or class II protein prediction over currently available predictor. In some embodiments, the method provided herein exhibits at least about a 30-fold improvement in a specific HLA class I or class II protein prediction over currently available predictor. In some embodiments, the method provided herein exhibits at least about a 40-fold improvement in a specific HLA class I or class II protein prediction over currently available predictor. In some embodiments, the method provided herein exhibits at least about a 50-fold improvement in a specific HLA class I or class II protein prediction over currently available predictor. In some embodiments, the method provided herein exhibits at least about a 60-fold improvement in a specific HLA class I or class II protein prediction over currently available predictor.

In one aspect, presented herein are methods of immunotherapy tailored or personalized for a specific subject. Every subject or patient expresses a specific array of HLA class I and HLA class II proteins. HLA typing is a well-known technique that allows determination of the specific repertoire of HLA proteins expressed by the subject. Once the HLA heterodimers expressed by a specific subject is known, having an improved, sophisticated and reliable method as described herein for predicting peptides that can bind to a specific HLA class I or class II molecule or complex, with high fidelity can ensure that a specific immune response can be generated tailored specifically for the subject.

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting. The terms “one or more” or “at least one,” such as one or more or at least one member(s) of a group of members, is clear per se, by means of further exemplification, the term encompasses inter alia a reference to any one of said members, or to any two or more of said members, such as, e.g., any >3, ≥4, ≥5, ≥6 or ≥7 etc. of said members, and up to all said members.

Reference in the specification to “some embodiments,” “an embodiment,” “one embodiment” or “other embodiments” means that a feature, structure, or characteristic described in connection with the embodiments is included in at least some embodiments, but not necessarily all embodiments, of the present disclosure.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the disclosure can be used to achieve methods of the disclosure.

The term “about” or “approximately” as used herein when referring to a measurable value such as a parameter, an amount, a temporal duration, and the like, is meant to encompass variations of +/−20% or less, +/−10% or less, +/−5% or less, or +/−1% or less of and from the specified value, insofar such variations are appropriate to perform in the present disclosure. It is to be understood that the value to which the modifier “about” or “approximately” refers is itself also specifically disclosed.

The term “immune response” includes T cell mediated and/or B cell mediated immune responses that are influenced by modulation of T cell costimulation. Exemplary immune responses include T cell responses, e.g., cytokine production, and cellular cytotoxicity. In addition, the term immune response includes immune responses that are indirectly affected by T cell activation, e.g., antibody production (humoral responses) and activation of cytokine responsive cells, e.g., macrophages.

A “receptor” is to be understood as meaning a biological molecule or a molecule grouping capable of binding a ligand. A receptor can serve to transmit information in a cell, a cell formation or an organism. The receptor comprises at least one receptor unit and can contain two or more receptor units, where each receptor unit can consist of a protein molecule, e.g., a glycoprotein molecule. The receptor has a structure that complements the structure of a ligand and can complex the ligand as a binding partner. Signaling information can be transmitted by conformational changes of the receptor following binding with the ligand on the surface of a cell. According to the present disclosure, a receptor can refer to proteins of MHC classes I and II capable of forming a receptor/ligand complex with a ligand, e.g., a peptide or peptide fragment of suitable length. The class I and class II MHC peptides that are encoded by HLA class I and class II alleles are often referred to here as HLA class I and HLA class II peptides respectively, or HLA class I and HLA class II peptides, or HLA class I class II proteins, or HLA class I and HLA class II proteins, or HLA class I and class II molecules, or such common variants thereof, as is well understood within the context of the discussion by one of ordinary skill in the art.

A “ligand” is a molecule which is capable of forming a complex with a receptor. According to the present disclosure, a ligand is to be understood as meaning, for example, a peptide or peptide fragment which has a suitable length and suitable binding motifs in its amino acid sequence, so that the peptide or peptide fragment is capable of binding to and forming a complex with proteins of MHC class I or MHC class II (i.e., HLA class I and HLA class II proteins).

An “antigen” is a molecule capable of stimulating an immune response, and can be produced by cancer cells or infectious agents or an autoimmune disease. Antigens recognized by T cells, whether helper T lymphocytes (T helper (TH) cells) or cytotoxic T lymphocytes (CTLs), are not recognized as intact proteins, but rather as small peptides in association with HLA class I or class II proteins on the surface of cells. During the course of a naturally occurring immune response, antigens that are recognized in association with HLA class II molecules on antigen presenting cells (APCs) are acquired from outside the cell, internalized, and processed into small peptides that associate with the HLA class II molecules. APCs can also cross-present peptide antigens by processing exogenous antigens and presenting the processed antigens on HLA class I molecules. Antigens that give rise to peptides that are recognized in association with HLA class I MHC molecules are generally peptides that are produced within the cells, and these antigens are processed and associated with class I MHC molecules. It is now understood that the peptides that associate with given HLA class I or class II molecules are characterized as having a common binding motif, and the binding motifs for a large number of different HLA class I and II molecules have been determined. Synthetic peptides that correspond to the amino acid sequence of a given antigen and that contain a binding motif for a given HLA class I or II molecule can also be synthesized. These peptides can then be added to appropriate APCs, and the APCs can be used to stimulate a T helper cell or CTL response either in vitro or in vivo. The binding motifs, methods for synthesizing the peptides, and methods for stimulating a T helper cell or CTL response are all known and readily available to one of ordinary skill in the art.

The term “peptide” is used interchangeably with “mutant peptide” and “neoantigenic peptide” in the present specification. Similarly, the term “polypeptide” is used interchangeably with “mutant polypeptide” and “neoantigenic polypeptide” in the present specification. By “neoantigen” or “neoepitope” is meant a class of tumor antigens or tumor epitopes which arises from tumor-specific mutations in expressed protein. The present disclosure further includes peptides that comprise tumor specific mutations, peptides that comprise known tumor specific mutations, and mutant polypeptides or fragments thereof identified by the method of the present disclosure. These peptides and polypeptides are referred to herein as “neoantigenic peptides” or “neoantigenic polypeptides.” The polypeptides or peptides can be a variety of lengths, either in their neutral (uncharged) forms or in forms which are salts, and either free of modifications such as glycosylation, side chain oxidation, phosphorylation, or any post-translational modification or containing these modifications, subject to the condition that the modification not destroy the biological activity of the polypeptides as herein described. In some embodiments, the neoantigenic peptides of the present disclosure can include: for HLA class I, 22 residues or less in length, e.g., from about 8 to about 22 residues, from about 8 to about 15 residues, or 9 or 10 residues; for HLA Class II, 40 residues or less in length, e.g., from about 8 to about 40 residues in length, from about 8 to about 24 residues in length, from about 12 to about 19 residues, or from about 14 to about 18 residues. In some embodiments, a neoantigenic peptide or neoantigenic polypeptide comprises a neoepitope.

The term “epitope” includes any protein determinant capable of specific binding to an antibody, antibody peptide, and/or antibody-like molecule (including but not limited to a T cell receptor) as defined herein. Epitopic determinants typically consist of chemically active surface groups of molecules such as amino acids or sugar side chains and generally have specific three-dimensional structural characteristics as well as specific charge characteristics.

A “T cell epitope” is a peptide sequence which can be bound by the MHC molecules of class I or II in the form of a peptide-presenting MHC molecule or MHC complex and then, in this form, be recognized and bound by cytotoxic T-lymphocytes or T-helper cells, respectively.

The term “antibody” as used herein includes IgG (including IgG1, IgG2, IgG3, and IgG4), IgA (including IgAQ1 and IgA2), IgD, IgE, IgM, and IgY, and is meant to include whole antibodies, including single-chain whole antibodies, and antigen-binding (Fab) fragments thereof. Antigen-binding antibody fragments include, but are not limited to, Fab, Fab′ and F(ab′)2, Fd (consisting of VH and CH1), single-chain variable fragment (scFv), single-chain antibodies, disulfide-linked variable fragment (dsFv) and fragments comprising either a VL or VH domain. The antibodies can be from any animal origin. Antigen-binding antibody fragments, including single-chain antibodies, can comprise the variable region(s) alone or in combination with the entire or partial of the following: hinge region, CH1, CH2, and CH3 domains. Also included are any combinations of variable region(s) and hinge region, CH1, CH2, and CH3 domains. Antibodies can be monoclonal, polyclonal, chimeric, humanized, and human monoclonal and polyclonal antibodies which, e.g., specifically bind an HLA-associated polypeptide or an HLA-HLA binding peptide (HLA-peptide) complex. A person of skill in the art will recognize that a variety of immunoaffinity techniques are suitable to enrich soluble proteins, such as soluble HLA-peptide complexes or membrane bound HLA-associated polypeptides, e.g., which have been proteolytically cleaved from the membrane. These include techniques in which (1) one or more antibodies capable of specifically binding to the soluble protein are immobilized to a fixed or mobile substrate (e.g., plastic wells or resin, latex or paramagnetic beads), and (2) a solution containing the soluble protein from a biological sample is passed over the antibody coated substrate, allowing the soluble protein to bind to the antibodies. The substrate with the antibody and bound soluble protein is separated from the solution, and optionally the antibody and soluble protein are disassociated, for example by varying the pH and/or the ionic strength and/or ionic composition of the solution bathing the antibodies. Alternatively, immunoprecipitation techniques in which the antibody and soluble protein are combined and allowed to form macromolecular aggregates can be used. The macromolecular aggregates can be separated from the solution by size exclusion techniques or by centrifugation.

The term “immunopurification (IP)” (or immunoaffinity purification or immunoprecipitation) is a process well known in the art and is widely used for the isolation of a desired antigen from a sample. In general, the process involves contacting a sample containing a desired antigen with an affinity matrix comprising an antibody to the antigen covalently attached to a solid phase. The antigen in the sample becomes bound to the affinity matrix through an immunochemical bond. The affinity matrix is then washed to remove any unbound species. The antigen is removed from the affinity matrix by altering the chemical composition of a solution in contact with the affinity matrix. The immunopurification can be conducted on a column containing the affinity matrix, in which case the solution is an eluent. Alternatively, the immunopurification can be in a batch process, in which case the affinity matrix is maintained as a suspension in the solution. An important step in the process is the removal of antigen from the matrix. This is commonly achieved by increasing the ionic strength of the solution in contact with the affinity matrix, for example, by the addition of an inorganic salt. An alteration of pH can also be effective to dissociate the immunochemical bond between antigen and the affinity matrix.

An “agent” is any small molecule chemical compound, antibody, nucleic acid molecule, or polypeptide, or fragments thereof.

An “alteration” or “change” is an increase or decrease. An alteration can be by as little as 1%, 2%, 3%, 4%, 5%, 10%, 20%, 30%, or by 40%, 50%, 60%, or even by as much as 70%, 75%, 80%, 90%, or 100%.

A “biologic sample” is any tissue, cell, fluid, or other material derived from an organism. As used herein, the term “sample” includes a biologic sample such as any tissue, cell, fluid, or other material derived from an organism. “Specifically binds” refers to a compound (e.g., peptide) that recognizes and binds a molecule (e.g., polypeptide), but does not substantially recognize and bind other molecules in a sample, for example, a biological sample.

“Capture reagent” refers to a reagent that specifically binds a molecule (e.g., a nucleic acid molecule or polypeptide) to select or isolate the molecule (e.g., a nucleic acid molecule or polypeptide).

As used herein, the terms “determining”, “assessing”, “assaying”, “measuring”, “detecting” and their grammatical equivalents refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

A “fragment” is a portion of a protein or nucleic acid that is substantially identical to a reference protein or nucleic acid. In some embodiments, the portion retains at least 50%, 75%, or 80%, or 90%, 95%, or even 99% of the biological activity of the reference protein or nucleic acid described herein.

The terms “isolated,” “purified”, “biologically pure” and their grammatical equivalents refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of the present disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications can give rise to different isolated proteins, which can be separately purified.

An “isolated” polypeptide (e.g., a peptide from an HLA-peptide complex) or polypeptide complex (e.g., an HLA-peptide complex) is a polypeptide or polypeptide complex of the present disclosure that has been separated from components that naturally accompany it. Typically, the polypeptide or polypeptide complex is isolated when it is at least 60%, by weight, free from the proteins and naturally-occurring organic molecules with which it is naturally associated. The preparation can be at least 75%, at least 90%, or at least 99%, by weight, a polypeptide or polypeptide complex of the present disclosure. An isolated polypeptide or polypeptide complex of the present disclosure can be obtained, for example, by extraction from a natural source, by expression of a recombinant nucleic acid encoding such a polypeptide or one or more components of a polypeptide complex, or by chemically synthesizing the polypeptide or one or more components of the polypeptide complex. Purity can be measured by any appropriate method, for example, column chromatography, polyacrylamide gel electrophoresis, or by HPLC analysis. In some cases, an HLA allele-encoded MHC Class II protein (i.e., an MHC class II peptide) is interchangeably referred to within this document as an HLA class II protein (or HLA class II peptide).

The term “vectors” refers to a nucleic acid molecule capable of transporting or mediating expression of a heterologous nucleic acid. A plasmid is a species of the genus encompassed by the term “vector.” A vector typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. In general, expression vectors of utility are often in the form of “plasmids” which refer to circular double stranded DNA molecules which, in their vector form are not bound to the chromosome, and typically comprise entities for stable or transient expression or the encoded DNA. Other expression vectors that can be used in the methods as disclosed herein include, but are not limited to plasmids, episomes, bacterial artificial chromosomes, yeast artificial chromosomes, bacteriophages or viral vectors, and such vectors can integrate into the host's genome or replicate autonomously in the cell. A vector can be a DNA or RNA vector. Other forms of expression vectors known by those skilled in the art which serve the equivalent functions can also be used, for example, self-replicating extrachromosomal vectors or vectors capable of integrating into a host genome. Exemplary vectors are those capable of autonomous replication and/or expression of nucleic acids to which they are linked.

The terms “spacer” or “linker” as used in reference to a fusion protein refers to a peptide that joins the proteins comprising a fusion protein. Generally, a spacer has no specific biological activity other than to join or to preserve some minimum distance or other spatial relationship between the proteins or RNA sequences. However, in some embodiments, the constituent amino acids of a spacer can be selected to influence some property of the molecule such as the folding, net charge, or hydrophobicity of the molecule. Suitable linkers for use in an embodiment of the present disclosure are well known to those of skill in the art and include, but are not limited to, straight or branched-chain carbon linkers, heterocyclic carbon linkers, or peptide linkers. The linker is used to separate two antigenic peptides by a distance sufficient to ensure that, in some embodiments, each antigenic peptide properly folds. Exemplary peptide linker sequences adopt a flexible extended conformation and do not exhibit a propensity for developing an ordered secondary structure. Typical amino acids in flexible protein regions include Gly, Asn and Ser.

Virtually any permutation of amino acid sequences containing Gly, Asn and Ser would be expected to satisfy the above criteria for a linker sequence. Other near neutral amino acids, such as Thr and Ala, also can be used in the linker sequence. Still other amino acid sequences that can be used as linkers are disclosed in Maratea et al. (1985), Gene 40: 39-46; Murphy et al. (1986) Proc. Nat'l. Acad. Sci. USA 83: 8258-62; U.S. Pat. Nos. 4,935,233; 4,751,180.

The term “neoplasia” refers to any disease that is caused by or results in inappropriately high levels of cell division, inappropriately low levels of apoptosis, or both. Glioblastoma is one non-limiting example of a neoplasia or cancer. The terms “cancer” or “tumor” or “hyperproliferative disorder” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. Cancers include, but are not limited to, B cell cancer (e.g., multiple myeloma, Waldenstrom's macroglobulinemia), the heavy chain diseases (such as, for example, alpha chain disease, gamma chain disease, and mu chain disease), benign monoclonal gammopathy, and immunocytic amyloidosis, melanomas, breast cancer, lung cancer, bronchus cancer, colorectal cancer, prostate cancer (e.g., metastatic, hormone refractory prostate cancer), pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers can be characterized in various other ways including, but not limited to, serous, endometrioid, mucinous, clear cell, brenner, or undifferentiated. In some embodiments, the present disclosure is used in the treatment, diagnosis, and/or prognosis of lymphoma or its subtypes, including, but not limited to, mantle cell lymphoma. Lymphoproliferative disorders are also considered to be proliferative diseases.

The term “vaccine” is to be understood as meaning a composition for generating immunity for the prophylaxis and/or treatment of diseases (e.g., neoplasia/tumor/infectious agents/autoimmune diseases). Accordingly, vaccines are medicaments which comprise antigens and are intended to be used in humans or animals for generating specific defense and protective substance by vaccination. A “vaccine composition” can include a pharmaceutically acceptable excipient, carrier or diluent. Aspects of the present disclosure relate to use of the technology in preparing an antigen-based vaccine. In these embodiments, vaccine is meant to refer one or more disease-specific antigenic peptides (or corresponding nucleic acids encoding them). In some embodiments, the antigen-based vaccine contains at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, or more antigenic peptides. In some embodiments, the antigen-based vaccine contains from 2 to 100, 2 to 75, 2 to 50, 2 to 25, 2 to 20, 2 to 19, 2 to 18, 2 to 17, 2 to 16, 2 to 15, 2 to 14, 2 to 13, 2 to 12, 2 to 10, 2 to 9, 2 to 8, 2 to 7, 2 to 6, 2 to 5, 2 to 4, 3 to 100, 3 to 75, 3 to 50, 3 to 25,3 to 20, 3 to 19, 3 to 18, 3 to 17, 3 to 16, 3 to 15,3 to 14, 3 to 13, 3 to 12, 3 to 10, 3 to 9, 3 to 8, 3 to 7, 3 to 6, 3 to 5, 4 to 100, 4 to 75, 4 to 50, 4 to 25, 4 to 20, 4 to 19, 4 to 18, 4 to 17, 4 to 16, 4 to 15, 4 to 14, 4 to 13, 4 to 12, 4 to 10, 4 to 9, 4 to 8, 4 to 7, 4 to 6, 5 to 100, 5 to 75, 5 to 50, 5 to 25, 5 to 20, 5 to 19, 5 to 18, 5 to 17, 5 to 16, 5 to 15,5 to 14,5 to 13, 5 to 12,5 to 10,5 to 9, 5 to 8, or 5 to 7 antigenic peptides. In some embodiments, the antigen-based vaccine contains 2, 3, 4, 5, 6, 7, 8, 9, 10, 11, 12, 13, 14, 15, 16, 17, 18, 19, or 20 antigenic peptides. In some cases, the antigenic peptides are neoantigenic peptides. In some cases, the antigenic peptides comprise one or more neoepitopes.

The term “pharmaceutically acceptable” refers to approved or approvable by a regulatory agency of the Federal or a state government or listed in the U.S. Pharmacopeia or other generally recognized pharmacopeia for use in animals, including humans. A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent. A “pharmaceutically acceptable salt” of pooled disease specific antigens as recited herein can be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluene sulfonic, methane sulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC-(CH2)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled disease specific antigens provided herein, including those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence, but will typically exhibit substantial identity. Polynucleotides having substantial identity to an endogenous sequence are typically capable of hybridizing with at least one strand of a double-stranded nucleic acid molecule. “Hybridize” refers to when nucleic acid molecules pair to form a double-stranded molecule between complementary polynucleotide sequences, or portions thereof, under various conditions of stringency. (See, e.g., Wahl, G. M. and S. L. Berger (1987) Methods Enzymol. 152:399; Kimmel, A. R. (1987) Methods Enzymol. 152:507). For example, stringent salt concentration can ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions can ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an exemplary embodiment, hybridization can occur at 30° C. in 750 mM NaCl, 75 mM trisodium citrate, and 1% SDS. In another exemplary embodiment, hybridization can occur at 37° C. in 500 mM NaCl, 50 mM trisodium citrate, 1% SDS, 35% formamide, and 100 μg/ml denatured salmon sperm DNA (ssDNA). In another exemplary embodiment, hybridization can occur at 42° C. in 250 mM NaCl, 25 mM trisodium citrate, 1% SDS, 50% formamide, and 200 μg/ml ssDNA. Useful variations on these conditions will be readily apparent to those skilled in the art. For most applications, washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps can be less than about 30 mM NaCl and 3 mM trisodium citrate, or less than about 15 mM NaCl and 1.5 mM trisodium citrate. Stringent temperature conditions for the wash steps can include a temperature of at least about 25° C., of at least about 42° C., or at least about 68° C. In exemplary embodiments, wash steps can occur at 25° C. in 30 mM NaCl, 3 mM trisodium citrate, and 0.10% SDS. In other exemplary embodiments, wash steps can occur at 42° C. in 15 mM NaCl, 1.5 mM trisodium citrate, and 0.10% SDS. In another exemplary embodiment, wash steps can occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Substantially identical” refers to a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence can be at least 60%, 80% or 85%, 90%, 95%, 96%, 97%, 98%, or even 99% or more identical at the amino acid level or nucleic acid to the sequence used for comparison. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program can be used, with a probability score between e-3 and e-m° indicating a closely related sequence. A “reference” is a standard of comparison.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

The terms “treat,” “treated,” “treating,” “treatment,” and the like are meant to refer to reducing, preventing, or ameliorating a disorder and/or symptoms associated therewith (e.g., a neoplasia or tumor or infectious agent or an autoimmune disease). “Treating” can refer to administration of the therapy to a subject after the onset, or suspected onset, of a disease (e.g., cancer or infection by an infectious agent or an autoimmune disease). “Treating” includes the concepts of “alleviating”, which refers to lessening the frequency of occurrence or recurrence, or the severity, of any symptoms or other ill effects related to the disease and/or the side effects associated with therapy. The term “treating” also encompasses the concept of “managing” which refers to reducing the severity of a disease or disorder in a patient, e.g., extending the life or prolonging the survivability of a patient with the disease, or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

The term “prevent”, “preventing”, “prevention” and their grammatical equivalents as used herein, means avoiding or delaying the onset of symptoms associated with a disease or condition in a subject that has not developed such symptoms at the time the administering of an agent or compound commences.

The term “therapeutic effect” refers to some extent of relief of one or more of the symptoms of a disorder (e.g., a neoplasia, tumor, or infection by an infectious agent or an autoimmune disease) or its associated pathology. “Therapeutically effective amount” as used herein refers to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian can start doses of the compounds of the present disclosure employed in a pharmaceutical composition at levels lower than that required in order to achieve the desired therapeutic effect and gradually increase the dosage until the desired effect is achieved. Disease, condition, and disorder are used interchangeably herein.

Those of ordinary skill in the art will recognize that the terms “peptide tag,” “affinity tag,” “epitope tag,” or “affinity acceptor tag” are used interchangeably herein. As used herein, the term “affinity acceptor tag” refers to an amino acid sequence that permits the tagged protein to be readily detected or purified, for example, by affinity purification. An affinity acceptor tag is generally (but need not be) placed at or near the N- or C-terminus of an HLA allele. Various peptide tags are well known in the art. Non-limiting examples include poly-histidine tag (e.g., 4 to 15 consecutive His residues (SEQ ID NO: 4), such as 8 consecutive His residues (SEQ ID NO: 5)); poly-histidine-glycine tag; HA tag (e.g., Field et al., Mol. Cell. Biol., 8:2159, 1988); c-myc tag (e.g., Evans et al., Mol. Cell. Biol., 5:3610, 1985); Herpes simplex virus glycoprotein D (gD) tag (e.g., Paborsky et al., Protein Engineering, 3:547, 1990); FLAG tag (e.g., Hopp et al., BioTechnology, 6:1204, 1988; U.S. Pat. Nos. 4,703,004 and 4,851,341); KT3 epitope tag (e.g., Martine et al., Science, 255:192, 1992); tubulin epitope tag (e.g., Skinner, Biol. Chem., 266:15173, 1991); T7 gene 10 protein peptide tag (e.g., Lutz-Freyemuth et al., Proc. Natl. Acad. Sci. USA, 87:6393, 1990); streptavidin tag (StrepTag.TM. or StrepTagII.TM; see, e.g., Schmidt et al., J. Mol. Biol., 255(5):753-766, 1996 or U.S. Pat. No. 5,506,121; also commercially available from Sigma-Genosys); or a VSV-G epitope tag derived from the Vesicular Stomatis viral glycoprotein; or a V5 tag derived from a small epitope (Pk) found on the P and V proteins of the paramyxovirus of simian virus 5 (SV5). In some embodiments, the affinity acceptor tag is an “epitope tag,” which is a type of peptide tag that adds a recognizable epitope (antibody binding site) to the HLA-protein to provide binding of corresponding antibody, thereby allowing identification or affinity purification of the tagged protein. Non-limiting example of an epitope tag is protein A or protein G, which binds to IgG. In some embodiments, the matrix of IgG Sepharose 6 Fast Flow chromatography resin is covalently coupled to human IgG. This resin allows high flow rates, for rapid and convenient purification of a protein tagged with protein A. Numerous other tag moieties are known to, and can be envisioned by, the ordinarily skilled artisan, and are contemplated herein. Any peptide tag can be used as long as it is capable of being expressed as an element of an affinity acceptor tagged HLA-peptide complex.

As used herein, the term “affinity molecule” refers to a molecule or a ligand that binds with chemical specificity to an affinity acceptor peptide. Chemical specificity is the ability of a protein's binding site to bind specific ligands. The fewer ligands a protein can bind, the greater its specificity. Specificity describes the strength of binding between a given protein and ligand. This relationship can be described by a dissociation constant (K_{D}), which characterizes the balance between bound and unbound states for the protein-ligand system.

The term “affinity acceptor tagged HLA-peptide complex” refers to a complex comprising an HLA class I or class II-associated peptide or a portion thereof specifically bound to a single allelic recombinant HLA class I or class II peptide comprising an affinity acceptor peptide.

The terms “specific binding” or “specifically binding” when used in reference to the interaction of an affinity molecule and an affinity acceptor tag or an epitope and an HLA peptide mean that the interaction is dependent upon the presence of a particular structure (e.g., the antigenic determinant or epitope) on the protein; in other words, the affinity molecule is recognizing and binding to a specific affinity acceptor peptide structure rather than to proteins in general.

As used herein, the term “affinity” refers to a measure of the strength of binding between two members of a binding pair, for example, an “affinity acceptor tag” and an “affinity molecule” and an HLA-binding peptide and an HLA class I or II molecule. K_{D} is the dissociation constant and has units of molarity. The affinity constant is the inverse of the dissociation constant. An affinity constant is sometimes used as a generic term to describe this chemical entity. It is a direct measure of the energy of binding. Affinity can be determined experimentally, for example by surface plasmon resonance (SPR) using commercially available Biacore SPR units. Affinity can also be expressed as the inhibitory concentration 50 (IC₅₀), that concentration at which 50% of the peptide is displaced. Likewise, InIC₅₀ refers to the natural log of the IC₅₀. K_{off} refers to the off-rate constant, for example, for dissociation of an affinity molecule from the affinity acceptor tagged HLA-peptide complex.

In some embodiments, an affinity acceptor tagged HLA-peptide complex comprises biotin acceptor peptide (BAP) and is immunopurified from complex cellular mixtures using streptavidin/NeutrAvidin beads. The biotin-avidin/streptavidin binding is the strongest non-covalent interaction known in nature. This property is exploited as a biological tool for a wide range of applications, such as immunopurification of a protein to which biotin is covalently attached. In an exemplary embodiment, the nucleic acid sequence encoding the HLA allele implements biotin acceptor peptide (BAP) as an affinity acceptor tag for immunopurification. BAP can be specifically biotinylated in vivo or in vitro at a single lysine residue within the tag (e.g., U.S. Pat. Nos. 5,723,584; 5,874,239; and 5,932,433; and U.K Pat. No. GB2370039). BAP is typically 15 amino acids long and contains a single lysine as a biotin acceptor residue. In some embodiments, BAP is placed at or near the N- or C-terminus of a single allele HLA peptide. In some embodiments, BAP is placed in between a heavy chain domain and β2 microglobulin domain of an HLA class I peptide. In some embodiments, BAP is placed in between β-chain domain and α-chain domain of an HLA class II peptide. In some embodiments, BAP is placed in loop regions between α1, α2, and α3 domains of the heavy chain of HLA class I, or between α1 and α2 and β1 and β2 domains of the α-chain and β3-chain, respectively of HLA class II.

As used herein, the term “biotin” refers to the compound biotin itself and analogues, derivatives and variants thereof. Thus, the term “biotin” includes biotin (cis-hexahydro-2-oxo-1H-thieno [3,4]imidazole-4-pentanoic acid) and any derivatives and analogs thereof, including biotin-like compounds. Such compounds include, for example, biotin-e-N-lysine, biocytin hydrazide, amino or sulfhydryl derivatives of 2-iminobiotin and biotinyl-E-aminocaproic acid-N-hydroxysuccinimide ester, sulfosuccinimideiminobiotin, biotinbromoacetylhydrazide, p-diazobenzoyl biocytin, 3-(N-maleimidopropionyl)biocytin, desthiobiotin, and the like. The term “biotin” also comprises biotin variants that can specifically bind to one or more of a Rhizavidin, avidin, streptavidin, tamavidin moiety, or other avidin-like peptides.

As used herein, a “PPV determination method” can refer to a presentation PPV determination method. For example, a “PPV determination method” can refer to a method comprising (a) processing amino acid information of a plurality of test peptide sequences using an HLA peptide presentation prediction model, such as a machine learning HLA peptide presentation prediction model, to generate a plurality of test presentation predictions, each test presentation prediction indicative of a likelihood that one or more proteins encoded by a class II HLA allele of a cell, such as a class II HLA allele of a cell of a subject, can present a given test peptide sequence of the plurality of test peptide sequences, wherein the plurality of test peptide sequences comprises at least 500 test peptide sequences comprising (i) at least one hit peptide sequence identified by mass spectrometry to be presented by an HLA protein expressed in cells and (ii) at least 499 decoy peptide sequences contained within a protein encoded by a genome of an organism, such as an organism that is the same species as the subject, wherein the plurality of test peptide sequences comprises a ratio of less than one of the number of hit peptide sequences to the number of decoy peptide sequences, such as a ratio of 1:499 of the at least one hit peptide sequences to the at least 499 decoy peptide sequences; (b) identifying or calling a top percentage of the plurality of test peptide sequences, such as a top 0.2% of the plurality of test peptide sequences, as being presented by the class II HLA allele of a cell; and (c) calculating a PPV of the HLA peptide presentation prediction model, wherein the PPV is the fraction of the test peptide sequences of the plurality that were identified or called as being presented by the class II HLA allele of a cell that are peptides observed by mass spectrometry as being presented by the class II HLA allele of a cell. In some embodiments, a decoy peptide is of the same length, i.e., comprises the same number of amino acids as a hit peptide. In some embodiments, a decoy peptide may comprise one more or one less amino acid as compared to the hit peptide. In some embodiments the decoy peptide is a peptide that is an endogenous peptide. In some embodiments a decoy peptide is a synthetic peptide. In some embodiments the decoy peptide is an endogenous peptide that has been identified by mass spectrometry to bind to a first MHC class I or class II protein, wherein the first MHC class I or class II protein is distinct from a second MHC class I or class II protein that binds to a hit peptide. In some embodiments, the decoy peptide may be a scrambled peptide, e.g., the decoy peptide may comprise an amino acid sequence in which the amino acid positions are rearranged relative to that of the hit peptide within the length of the peptide. In some embodiments, the PPV determination method can be a presentation PPV determination method.

In some embodiments, the ratio of the number of hit peptide sequences to the number of decoy peptide sequences is about 1:10, 1:20, 1:50, 1:100, 1:250, 1:500, 1:1000, 1:1500, 1:2000, 1:2500, 1:5000, 1:7500, 1:10000, 1:25000, 1:50000 or 1:100000. In some embodiments, the at least one hit peptide sequence comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 hit peptide sequences. In some embodiments, the at least 499 decoy peptide sequences comprises at least 500 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 52500, 55000, 57500, 60000, 62500, 65000, 67500, 70000, 72500, 75000, 77500, 80000, 82500, 85000, 87500, 90000, 92500, 95000, 97500, 100000, 125000, 150000, 175000, 200000, 225000, 250000, 275000, 300000, 325000, 350000, 375000, 400000, 425000, 450000, 475000, 500000, 600000, 700000, 800000, 900000 or 1000000 decoy peptide sequences. In some embodiments, the at least 500 test peptide sequences comprises at least 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 52500, 55000, 57500, 60000, 62500, 65000, 67500, 70000, 72500, 75000, 77500, 80000, 82500, 85000, 87500, 90000, 92500, 95000, 97500, 100000, 125000, 150000, 175000, 200000, 225000, 250000, 275000, 300000, 325000, 350000, 375000, 400000, 425000, 450000, 475000, 500000, 600000, 700000, 800000, 900000 or 1000000 test peptide sequences. In some embodiments, identifying or calling atop percentage of the plurality of test peptide sequences as being presented by the class II HLA allele of a cell comprises identifying or calling a top 0.20%, 0.30%, 0.40%, 0.50%, 0.60%, 0.70%, 0.80%, 0.90%, 1.00%, 1.10%, 1.20%, 1.30%, 1.40%, 1.50%, 1.60%, 1.70%, 1.80%, 1.90%, 2.00%, 2.10%, 2.20%, 2.30%, 2.40%, 2.50%, 2.60%, 2.70%, 2.80%, 2.90%, 3.00%, 3.10%, 3.20%, 3.30%, 3.40%, 3.50%, 3.60%, 3.70%, 3.80%, 3.90%, 4.00%, 4.10%, 4.20%, 4.30%, 4.40%, 4.50%, 4.60%, 4.70%, 4.80%, 4.90%, 5.00%, 5.10%, 5.20%, 5.30%, 5.40%, 5.50%, 5.60%, 5.70%, 5.80%, 5.90%, 6.00%, 6.10%, 6.20%, 6.30%, 6.40%, 6.50%, 6.60%, 6.70%, 6.80%, 6.90%, 7.00%, 7.10%, 7.20%, 7.30%, 7.40%, 7.50%, 7.60%, 7.70%, 7.80%, 7.90%, 8.00%, 8.10%, 8.20%, 8.30%, 8.40%, 8.50%, 8.60%, 8.70%, 8.80%, 8.90%, 9.00%, 9.10%, 9.20%, 9.30%, 9.40%, 9.50%, 9.60%, 9.70%, 9.80%, 9.90%, 10%, 11%, 12%, 13%, 14%, 15%, 16%, 17%, 18%, 19% or 20% as being presented by the class II HLA allele of a cell. In some embodiments, the cell is a mono-allelic cell.

As used herein, a “PPV determination method” can refer to a binding PPV determination method. For example, a “PPV determination method” can refer to a method comprising (a) processing amino acid information of a plurality of test peptide sequences using an HLA peptide binding prediction model, such as a machine learning HLA peptide binding prediction model, to generate a plurality of test binding predictions, each test binding prediction indicative of a likelihood that the one or more proteins encoded by a class I or class II HLA allele of a cell, such as a class I or class II HLA allele of a cell of a subject, binds to a given test peptide sequence of the plurality of test peptide sequences, wherein the plurality of test peptide sequences comprises at least 20 test peptide sequences comprising (i) at least one hit peptide sequence identified by mass spectrometry to be presented by an HLA protein expressed in cells and (ii) at least 19 decoy peptide sequences contained within a protein comprising at least one peptide sequence identified by mass spectrometry to be presented by an HLA protein expressed in cells, wherein the plurality of test peptide sequences comprises a ratio of less than one of the number of hit peptide sequences to the number of decoy peptide sequences, such as a ratio of 1:19 of the at least one hit peptide sequences to the at least 19 decoy peptide sequences; (b) identifying or calling a top percentage of the plurality of test peptide sequences, such as a top 5% of the plurality of test peptide sequences, as binding to the HLA protein; and (c) calculating a PPV of the HLA peptide binding prediction model, wherein the PPV is the fraction of the test peptide sequences of the plurality that were identified or called as binding to the class I or class II HLA allele of a cell that are peptides observed by mass spectrometry as being presented by the class I or class II HLA allele of a cell. In some embodiments, the ratio of the number of hit peptide sequences to the number of decoy peptide sequences is about 1:2, 1:3, 1:4, 1:5, 1:10, 1:20, 1:25, 1:30, 1:40, 1:50, 1:75, 1:100, 1:200, 1:250, 1:500 or 1:1000. In some embodiments, the at least one hit peptide sequence comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 hit peptide sequences. In some embodiments, the at least 19 decoy peptide sequences comprises at least 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 52500, 55000, 57500, 60000, 62500, 65000, 67500, 70000, 72500, 75000, 77500, 80000, 82500, 85000, 87500, 90000, 92500, 95000, 97500, 100000, 125000, 150000, 175000, 200000, 225000, 250000, 275000, 300000, 325000, 350000, 375000, 400000, 425000, 450000, 475000, 500000, 600000, 700000, 800000, 900000 or 1000000 decoy peptide sequences. In some embodiments, the at least 20 test peptide sequences comprises at least 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 52500, 55000, 57500, 60000, 62500, 65000, 67500, 70000, 72500, 75000, 77500, 80000, 82500, 85000, 87500, 90000, 92500, 95000, 97500, 100000, 125000, 150000, 175000, 200000, 225000, 250000, 275000, 300000, 325000, 350000, 375000, 400000, 425000, 450000, 475000, 500000, 600000, 700000, 800000, 900000 or 1000000 test peptide sequences. In some embodiments, identifying or calling a top percentage of the plurality of test peptide sequences as being presented by the class II HLA allele of a cell comprises identifying or calling a top 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% as being presented by the class II HLA allele of a cell. In some embodiments, the cell is a mono-allelic cell.

### Human Leukocyte Antigen (HLA) System

The immune system can be classified into two functional subsystems: the innate and the adaptive immune system. The innate immune system is the first line of defense against infections, and most potential pathogens are rapidly neutralized by this system before they can cause, for example, a noticeable infection. The adaptive immune system reacts to molecular structures, referred to as antigens, of the intruding organism. Unlike the innate immune system, the adaptive immune system is highly specific to a pathogen. Adaptive immunity can also provide long-lasting protection; for example, someone who recovers from measles is now protected against measles for their lifetime. There are two types of adaptive immune reactions, which include the humoral immune reaction and the cell-mediated immune reaction. In the humoral immune reaction, antibodies secreted by B cells into bodily fluids bind to pathogen-derived antigens, leading to the elimination of the pathogen through a variety of mechanisms, e.g. complement-mediated lysis. In the cell-mediated immune reaction, T cells capable of destroying other cells are activated. For example, if proteins associated with a disease are present in a cell, they are fragmented proteolytically to peptides within the cell. Specific cell proteins then attach themselves to the antigen or peptide formed in this manner and transport them to the surface of the cell, where they are presented to the molecular defense mechanisms, in T cells, of the body. Cytotoxic T cells recognize these antigens and kill the cells that harbor the antigens.

The term “major histocompatibility complex (MHC)”, “MHC molecules”, or “MHC proteins” refers to proteins capable of binding peptides resulting from the proteolytic cleavage of protein antigens and representing potential T cell epitopes, transporting them to the cell surface and presenting the peptides to specific cells, e.g., in cytotoxic T-lymphocytes or T-helper cells.

The human MHC is also called the HLA complex. Thus, the term “human leukocyte antigen (HLA) system”, “HLA molecules” or “HLA proteins” refers to a gene complex encoding the MHC proteins in humans. The term MHC is referred as the “H-2” complex in murine species. Those of ordinary skill in the art will recognize that the terms “major histocompatibility complex (MHC)”, “MHC molecules”, “MHC proteins” and “human leukocyte antigen (HLA) system”, “HLA molecules”, “HLA proteins” are used interchangeably herein.

HLA proteins are classified into two types, referred to as HLA class I and HLA class II. The structures of the proteins of the two HLA classes are very similar; however, they have very different functions. HLA class I proteins are present on the surface of almost all cells of the body, including most tumor cells. HLA class I proteins are loaded with antigens that usually originate from endogenous proteins or from pathogens present inside cells and are then presented to naïve or cytotoxic T-lymphocytes (CTLs). HLA class II proteins are present on antigen presenting cells (APCs), including but not limited to dendritic cells, B cells, and macrophages. They mainly present peptides, which are processed from external antigen sources, e.g. outside of the cells, to helper T cells. Most of the peptides bound by the HLA class I proteins originate from cytoplasmic proteins produced in the healthy host cells of an organism itself, and do not normally stimulate an immune reaction.

HLA class I molecules consist of two non-covalently linked polypeptide chains, an HLA-encoded a chain (heavy chain, 44 to 47 kD) and a non-HLA encoded subunit called β2 microglobulin (or, β2m), (12 kD). The α chain has three extracellular domains, α1, α2 and α3 and a transmembrane region, of which the α1 and α2 regions are capable of binding a peptide of about 7 to 13 amino acids (e.g., about 8 to 11 amino acids, or 9 or 10 amino acids). An HLA class 1 molecule binds to a peptide that has the suitable binding motifs, and presents it to cytotoxic T-lymphocytes. HLA class 1 heavy chains can be the protein product of an HLA-A allele, also termed as an HLA-A monomer, or the protein product of HLA-B allele (likewise, an HLA-B monomer) or the protein product of HLA-C allele (an HLA-C monomer), each of which complexes with a β-2-microglobulin. The α1 rests upon the non-HLA protein β2m; β2m is encoded by beta-2-microglobulin gene located on human chromosome 15. The α3 domain is connected to the transmembrane region, anchoring the HLA class I molecule to the cell membrane.

The peptide being presented is held by the floor of the peptide-binding groove, in the central region of the α1/α2 heterodimer (a molecule composed of two non-identical subunits). HLA class I-A, HLA class I-B or HLA class I-C are highly polymorphic. Each of a HLA class 1-A gene (termed HLA-A gene), a HLA class 1-B gene (termed HLA-B gene) and a HLA class 1-C gene (termed HLA-C gene) contains 8 exons, exon 1 encodes the leader peptide, exons 2 and 3 encode the α1 and α2 domains, exon 5 encodes the transmembrane region and exons 6 and 7 encode the cytoplasmic tail. Polymorphisms of exon 2 and exon 3 are responsible for the peptide binding specificity of each class 1 molecule. HLA class I-B gene (HLA-B) has many possible variations, expression patterns and presented antigens. This group is subdivided into a group encoded within HLA loci, e.g., HLA-E, HLA-F, HLA-G, as well as those not, e.g., stress ligands such as ULBPs, Rael and H60. The antigen/ligand for many of these molecules remains unknown, but they can interact with each of CD8+ T cells, NKT cells, and NK cells.

In some embodiments, the present disclosure utilizes a non-classical HLA class I-E allele. HLA-E molecules are recognized by natural killer (NK) cells and CD8+ T cells. HLA-E is expressed in almost all tissues including lung, liver, skin and placental cells. HLA-E expression is also detected in solid tumors (e.g., osteosarcoma and melanoma). HLA-E molecule binds to TCR expressed on CD8+ T cells, resulting in T cell activation. HLA-E is also known to bind CD94/NKG2 receptor expressed on NK cells and CD8+ T cells. CD94 can pair with several different isoforms of NKG2 to form receptors with potential to either inhibit (NKG2A, NKG2B) or promote (NKG2C) cellular activation. HLA-E can bind to a peptide derived from amino acid residues 3-11 of the leader sequences of most HLA-A, —B, —C, and -G molecules, but cannot bind to its own leader peptide. HLA-E has also been shown to present peptides derived from endogenous proteins similar to HLA-A, —B, and -C alleles. Under physiological conditions, the engagement of CD94/NKG2A with HLA-E, loaded with peptides from the HLA class I leader sequences, usually induces inhibitory signals. Cytomegalovirus (CMV) utilizes the mechanism for escape from NK cell immune surveillance via expression of the UL40 glycoprotein, mimicking the HLA-A leader. However, it is also reported that CD8+ T cells can recognize HLA-E loaded with the UL40 peptide derived from CMV Toledo strain and play a role in defense against CMV. A number of studies revealed several important functions of HLA-E in infectious disease and cancer.

The peptide antigens attach themselves to the molecules of HLA class I by competitive affinity binding within the endoplasmic reticulum before they are presented on the cell surface. Here, the affinity of an individual peptide antigen is directly linked to its amino acid sequence and the presence of specific binding motifs in defined positions within the amino acid sequence. If the sequence of such a peptide is known, it is possible to manipulate the immune system against diseased cells using, for example, peptide vaccines.

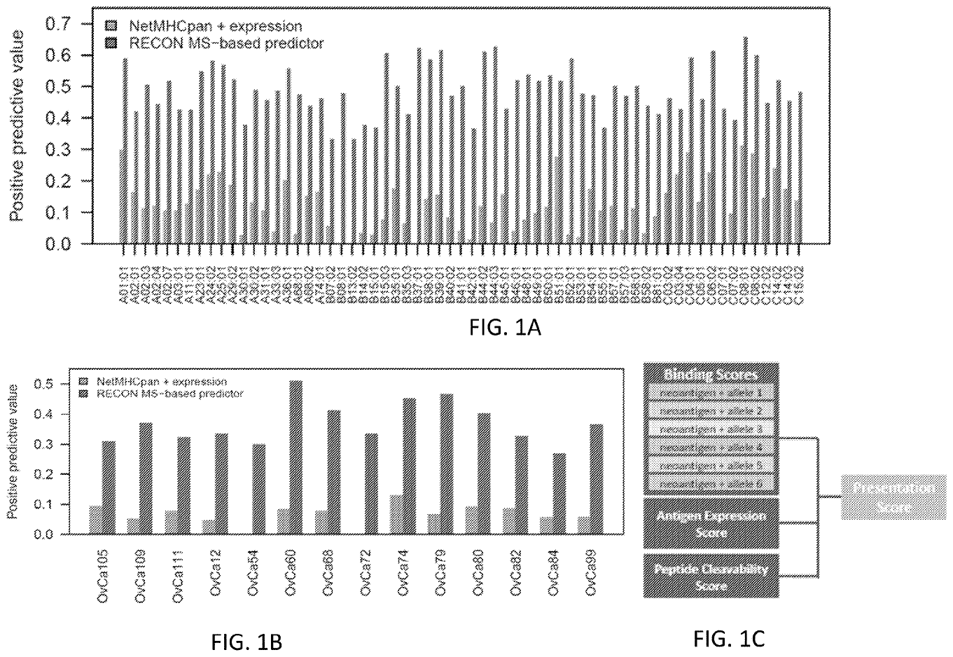

MHC molecules are highly polymorphic, that is, there are many MHC variants. Each variant is encoded by a variation of the gene encoding the protein, and each such variant gene is called an allele. For human beings, MHC is known as Human Leukocyte Antigens (HLA), which involves three types of HLA class II molecules: DP, DQ and DR. HLA class II peptides (FIG. 1) have two chains, α and β, each having two domains—α1 and α2 and β1 and β2—each chain having a transmembrane domain, α2 and β2, respectively, anchoring the HLA class II molecule to the cell membrane. The peptide-binding groove is formed from the heterodimer of α1 and β1. The most widely studied HLA-DR molecules have DRA and DRB, corresponding to α and β domains, respectively. The DRB is diverse, DRA is almost identical. Thus, the binding specificity of a DRB allele indicates that of the corresponding HLA-DR. Each MHC protein has its own binding specificity, meaning that a set of peptides binding to an MHC molecule can be different from those to another MHC molecule. Classic molecules present peptides to CD4+ lymphocytes. Nonclassic molecules, accessories, with intracellular functions, are not exposed on cell membranes but in internal membranes in lysosomes, normally loading the antigenic peptides onto classic HLA class II molecules.

In HLA class II system, phagocytes such as macrophages and immature dendritic cells take up entities by phagocytosis into phagosomes—though B cells exhibit the more general endocytosis into endosomes—which fuse with lysosomes whose acidic enzymes cleave the uptaken protein into many different peptides. Autophagy is another source of HLA class II peptides. Via physicochemical dynamics in molecular interaction with the HLA class II variants borne by the host, encoded in the host's genome, a particular peptide exhibits immunodominance and loads onto HLA class II molecules. These are trafficked to and externalized on the cell surface. The most studied subclasses of HLA class II genes are: HLA-DPA1, HLA-DPB1, HLA-DQA1, HLA-DQB1, HLA-DRA, and HLA-DRB1.

Presentation of peptides by HLA class II molecules to CD4+ helper T cells is required for immune responses to foreign antigens (Roche and Furuta, 2015). Once activated, CD4+ T cells promote B cell differentiation and antibody production, as well as CD8+ T cell (CTL) responses. CD4+ T cells also secrete cytokines and chemokines that activate and induce differentiation of other immune cells. HLA class II molecules are heterodimers of α—and β-chains that interact to form a peptide-binding groove that is more open than HLA class I peptide-binding grooves (Unanue et al., 2016). Peptides bound to HLA class II molecules are believed to have a 9-amino acid binding core with flanking residues on either N- or C-terminal side that overhang from the groove (Jardetzky et al., 1996; Stem et al., 1994). These peptides are usually 12-16 amino acids in length and often contain 3-4 anchor residues at positions P1, P4, P6/7 and P9 of the binding register (Rossjohn et al., 2015).

HLA alleles are expressed in codominant fashion, meaning that the alleles (variants) inherited from both parents are expressed equally. For example, each person carries 2 alleles of each of the 3 class I genes, (HLA-A, HLA-B and HLA-C) and so can express six different types of HLA class II. In the HLA class II locus, each person inherits a pair of HLA-DP genes (DPA1 and DPB1, which encode α and β chains), HLA-DQ (DQA1 and DQB1, for α and β chains), one gene HLA-DRα (DRA1), and one or more genes HLA-DRβ (DRB1 and DRB3, −4 or −5). HLA-DRB1, for example, has more than nearly 400 known alleles. That means that one heterozygous individual can inherit six or eight functioning HLA class II alleles: three or more from each parent. Thus, the HLA genes are highly polymorphic; many different alleles exist in the different individuals inside a population. Genes encoding HLA proteins have many possible variations, allowing each person's immune system to react to a wide range of foreign invaders. Some HLA genes have hundreds of identified versions (alleles), each of which is given a particular number. In some embodiments, the HLA class I alleles are HLA-A*02:01, HLA-B*14:02, HLA-A*23:01, HLA-E*01:01 (non-classical). In some embodiments, HLA class II alleles are HLA-DRB*01:01, HLA-DRB*01:02, HLA-DRB*11:01, HLA-DRB*15:01, and HLA-DRB*07:01.

Subject specific HLA alleles or HLA genotype of a subject can be determined by any method known in the art. In exemplary embodiments, HLA genotypes are determined by any method described in International Patent Application number PCT/US2014/068746, published Jun. 11, 2015 as WO2015085147, which is incorporated herein by reference in its entirety. Briefly, the methods include determining polymorphic gene types that can comprise generating an alignment of reads extracted from a sequencing data set to a gene reference set comprising allele variants of the polymorphic gene, determining a first posterior probability or a posterior probability derived score for each allele variant in the alignment, identifying the allele variant with a maximum first posterior probability or posterior probability derived score as a first allele variant, identifying one or more overlapping reads that aligned with the first allele variant and one or more other allele variants, determining a second posterior probability or posterior probability derived score for the one or more other allele variants using a weighting factor, identifying a second allele variant by selecting the allele variant with a maximum second posterior probability or posterior probability derived score, the first and second allele variant defining the gene type for the polymorphic gene, and providing an output of the first and second allele variant.

In some embodiments the MHC class II peptide: antigenic peptide binding and presenting prediction methods described herein have the capacity to predict binders from a large repertoire MHC class II peptides encoded by individual HLA alleles. In some embodiments, the MAPTAC technology is trained with a large database of mass spectrometry validated HLA-matched peptides. In some embodiments, the large database of mass spectrometry validated HLA-matched peptides comprise greater than 1.2×10\hat{ }6 such HLA-matched peptides. In some embodiments, the large database of mass spectrometry validated HLA-matched peptides cover greater than 150 HLA alleles including both MHC Class I and Class II allelic subtypes. In some embodiments, the database covers at least 95% of US population for HLA-I and HLA-II (DR subtype).

As described herein, there is a large body of evidence in both animals and humans that mutated epitopes are effective in inducing an immune response and that cases of spontaneous tumor regression or long term survival correlate with CD8+ T cell responses to mutated epitopes and that “immunoediting” can be tracked to alterations in expression of dominant mutated antigens in mice and man.

Sequencing technology has revealed that each tumor contains multiple, patient-specific mutations that alter the protein coding content of a gene. Such mutations create altered proteins, ranging from single amino acid changes (caused by missense mutations) to additions of long regions of novel amino acid sequences due to frame shifts, read-through of termination codons or translation of intron regions (novel open reading frame mutations; neoORFs). These mutated proteins are valuable targets for the host's immune response to the tumor as, unlike native proteins, they are not subject to the immune-dampening effects of self-tolerance. Therefore, mutated proteins are more likely to be immunogenic and are also more specific for the tumor cells compared to normal cells of the patient. In essence, short peptides (8-24 amino acids long) containing a cancer associated mutation are candidates for cancer immunotherapy.

In some embodiments the algorithm driving the prediction method can be further utilized for mutation calling on a peptide. In some embodiments, the prediction method may be used for determining driver mutation status, and/or RNA expression status, and/or cleavage prediction within the peptide.

The term “T cell” includes CD4+ T cells and CD8+ T cells. The term T cell also includes both T helper 1 type T cells and T helper 2 type T cells. T cells as used herein are generally classified by function and cell surface antigens (cluster differentiation antigens, or CDs), which also facilitate T cell receptor binding to antigen, into two major classes: helper T (TH) cells and cytotoxic T-lymphocytes (CTLs).

Mature helper T (TH) cells express the surface protein CD4 and are referred as CD4+ T cells. Following T cell development, matured, naïve T cells leave the thymus and begin to spread throughout the body, including the lymph nodes. Naïve T cells are those T cells that have never been exposed to the antigen that they are programmed to respond to. Like all T cells, they express the T cell receptor-CD3 complex. The T cell receptor (TCR) consists of both constant and variable regions. The variable region determines what antigen the T cell can respond to. CD4+ T cells have TCRs with an affinity for MHC class II, proteins and CD4 are involved in determining MHC affinity during maturation in the thymus. MHC class II proteins are generally only found on the surface of specialized antigen-presenting cells (APCs). Specialized antigen presenting cells (APCs) are primarily dendritic cells, macrophages and B cells, although dendritic cells are the only cell group that expresses MHC Class II constitutively (at all times). Some APCs also bind native (or unprocessed) antigens to their surface, such as follicular dendritic cells, but unprocessed antigens do not interact with T cells and are not involved in their activation. The peptide antigens that bind to HLA class I proteins are typically shorter than peptide antigens that bind to HLA class II proteins.

Cytotoxic T-lymphocytes (CTLs), also known as cytotoxic T cells, cytolytic T cells, CD8+ T cells, or killer T cells, refer to lymphocytes which induce apoptosis in targeted cells. CTLs form antigen-specific conjugates with target cells via interaction of TCRs with processed antigen (Ag) on target cell surfaces, resulting in apoptosis of the targeted cell. Apoptotic bodies are eliminated by macrophages. The term “CTL response” is used to refer to the primary immune response mediated by CTL cells. Cytotoxic T-lymphocytes have both T cell receptors (TCR) and CD8 molecules on their surface. T cell receptors are capable of recognizing and binding peptides complexed with the molecules of HLA class I. Each cytotoxic T-lymphocyte expresses a unique T cell receptor which is capable of binding specific MHC/peptide complexes. Most cytotoxic T cells express T cell receptors (TCRs) that can recognize a specific antigen. In order for the TCR to bind to the HLA class I molecule, the former must be accompanied by a glycoprotein called CD8, which binds to the constant portion of the HLA class I molecule. Therefore, these T cells are called CD8+ T cells. The affinity between CD8 and the MHC molecule keeps the T cell and the target cell bound closely together during antigen-specific activation. CD8+ T cells are recognized as T cells once they become activated and are generally classified as having a pre-defined cytotoxic role within the immune system. However, CD8+ T cells also have the ability to make some cytokines.

“T cell receptors (TCR)” are cell surface receptors that participate in the activation of T cells in response to the presentation of antigen. The TCR is generally made from two chains, alpha and beta, which assemble to form a heterodimer and associates with the CD3-transducing subunits to form the T cell receptor complex present on the cell surface. Each alpha and beta chain of the TCR consists of an immunoglobulin-like N-terminal variable (V) and constant (C) region, a hydrophobic transmembrane domain, and a short cytoplasmic region. As for immunoglobulin molecules, the variable regions of the alpha and beta chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of GVHD. It has been shown that normal surface expression of the TCR depends on the coordinated synthesis and assembly of all seven components of the complex (Ashwell and Klusner 1990). The inactivation of TCRα or TCRβ can result in the elimination of the TCR from the surface of T cells preventing recognition of alloantigen and thus GVHD.

However, TCR disruption generally results in the elimination of the CD3 signaling component and alters the means of further T cell expansion.

The term “HLA peptidome” refers to a pool of peptides which specifically interacts with a particular HLA class and can encompass thousands of different sequences. HLA peptidomes include a diversity of peptides, derived from both normal and abnormal proteins expressed in the cells. Thus, the HLA peptidomes can be studied to identify cancer specific peptides, for development of tumor immunotherapeutics and as a source of information about protein synthesis and degradation schemes within the cancer cells. In some embodiments, HLA peptidome is a pool of soluble HLA peptides (sHLA). In some embodiments, HLA peptidome is a pool of membrane associated HLA (mHLA).

“Antigen presenting cell” or “APC” includes professional antigen presenting cells (e.g., B lymphocytes, macrophages, monocytes, dendritic cells, Langerhans cells), as well as other antigen presenting cells (e.g., keratinocytes, endothelial cells, astrocytes, fibroblasts, oligodendrocytes, thymic epithelial cells, thyroid epithelial cells, glial cells (brain), pancreatic beta cells, and vascular endothelial cells). An “antigen presenting cell” or “APC” is a cell that expresses the Major Histocompatibility complex (MHC) molecules and can display foreign antigen complexed with MHC on its surface.

### Mono-Allelic HLA Cell Lines

A mono-allelic cell line expressing either a single HLA class I allele, a single pair of HLA class II alleles, or a single HLA class I allele and a single pair of HLA class II alleles can be generated by transducing or transfecting a suitable cell population with a polynucleic acid, e.g., a vector, coding a single HLA allele. Suitable cell populations include, e.g., HLA class I deficient cells lines in which a single HLA class I allele is exogenously expressed, HLA class II deficient cell lines in which a single exogenous pair of HLA class II alleles are expressed, or class I and class II deficient cell lines in which a single HLA class I and/or single pair of class II alleles are exogenously expressed. As an exemplary embodiment, the HLA class I deficient B cell line is B721.221. However, it is clear to a skilled person that other cell populations can be generated which are HLA class I and/or HLA class II deficient. An exemplary method for deleting/inactivating endogenous HLA class I or HLA class II genes includes CRISPR-Cas9 mediated genome editing in, for example, THP-1 cells. In some embodiments, the populations of cells are professional antigen presenting cells, such as macrophages, B cells, and dendritic cells.

The cells can be B cells or dendritic cells. In some embodiments, the cells are tumor cells or cells from a tumor cell line. In some embodiments, the cells are isolated from a patient. In some embodiments, the cells contain an infectious agent or a portion thereof. In some embodiments, the population of cells comprises at least 107 cells. In some embodiments, the population of cells are further modified, such as by increasing or decreasing the expression and/or activity of at least one gene. In some embodiments, the gene encodes a member of the immunoproteasome. The immunoproteasome is known to be involved in the processing of HLA class I binding peptides and includes the LMP2 (βli), MECL-1 (β2i), and LMP7 (β5i) subunits. The immunoproteasome can also be induced by interferon-gamma. Accordingly, in some embodiments, the population of cells can be contacted with one or more cytokines, growth factors, or other proteins. The cells can be stimulated with inflammatory cytokines such as interferon-gamma, IL-10, IL-6, and/or TNF-α. The population of cells can also be subjected to various environmental conditions, such as stress (heat stress, oxygen deprivation, glucose starvation, DNA damaging agents, etc.). In some embodiments, the cells are contacted with one or more of a chemotherapy drug, radiation, targeted therapies, or immunotherapy. The methods disclosed herein can therefore be used to study the effect of various genes or conditions on HLA peptide processing and presentation. In some embodiments, the conditions used are selected so as to match the condition of the patient for which the population of HLA-peptides is to be identified.

A single HLA-allele of the present disclosure can be encoded and expressed using a viral based system (e.g., an adenovirus system, an adeno associated virus (AAV) vector, a poxvirus, or a lentivirus). Plasmids that can be used for adeno associated virus, adenovirus, and lentivirus delivery have been described previously (see e.g., U.S. Pat. Nos. 6,955,808 and 6,943,019, and U.S. Patent application No. 20080254008, hereby incorporated by reference). Among vectors that can be used in the practice of the present disclosure, integration in the host genome of a cell is possible with retrovirus gene transfer methods, often resulting in long term expression of the inserted transgene. In an exemplary embodiment, the retrovirus is a lentivirus. Additionally, high transduction efficiencies have been observed in many different cell types and target tissues. The tropism of a retrovirus can be altered by incorporating foreign envelope proteins, expanding the potential target population of target cells. A retrovirus can also be engineered to allow for conditional expression of the inserted transgene, such that only certain cell types are infected by the lentivirus. Cell type specific promoters can be used to target expression in specific cell types. Lentiviral vectors are retroviral vectors (and hence both lentiviral and retroviral vectors can be used in the practice of the present disclosure). Moreover, lentiviral vectors are able to transduce or infect non-dividing cells and typically produce high viral titers.

Selection of a retroviral gene transfer system can depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the desired nucleic acid into the target cell to provide permanent expression. Widely used retroviral vectors that can be used in the practice of the present disclosure include those based upon murine leukemia virus (MuLV), gibbon ape leukemia virus (GaLV), Simian Immunodeficiency virus (SIV), human immunodeficiency virus (HIV), and combinations thereof (see, e.g., Buchscher et al., (1992) J. Virol. 66:2731-2739; Johann et al., (1992) J. Virol. 66:1635-1640; Sommnerfelt et al., (1990) Virol. 176:58-59; Wilson et al., (1998) J. Virol. 63:2374-2378; Miller et al., (1991) J. Virol. 65:2220-2224; PCT/US94/05700). Also, useful in the practice of the present disclosure is a minimal non-primate lentiviral vector, such as a lentiviral vector based on the equine infectious anemia virus (EIAV) (see, e.g., Balagaan, (2006) J Gene Med; 8: 275-285, Published online 21 Nov. 2005 in Wiley InterScience DOI: 10.1002/jgm.845). The vectors can have cytomegalovirus (CMV) promoter driving expression of the target gene. Accordingly, the present disclosure contemplates amongst vector(s) useful in the practice of the present disclosure: viral vectors, including retroviral vectors and lentiviral vectors.

Any HLA allele can be expressed in the cell population. In an exemplary embodiment, the HLA allele is an HLA class I allele. In some embodiments, the HLA class I allele is an HLA-A allele or an HLA-B allele. In some embodiments, the HLA allele is an HLA class II allele. Sequences of HLA class I and class II alleles can be found in the IPD-IMGT/HLA Database. Exemplary HLA alleles include, but are not limited to, HLA-A*02:01, HLA-B*14:02, HLA-A*23:01, HLA-E*01:01, HLA-DRB*01:01, HLA-DRB*01:02, HLA-DRB*11:01, HLA-DRB*15:01, and HLA-DRB*07:01.

In some embodiments, the HLA allele is selected so as to correspond to a genotype of interest. In some embodiments, the HLA allele is a mutated HLA allele, which can be non-naturally occurring allele or a naturally occurring allele in an afflicted patient. The methods disclosed herein have the further advantage of identifying HLA binding peptides for HLA alleles associated with various disorders as well as alleles which are present at low frequency.

Accordingly, in some embodiments, the method provided herein can identify the HLA allele even if it is present at a frequency of less than 1% within a population, such as within the Caucasian population.

In some embodiments, the nucleic acid sequence encoding the HLA allele further comprises an affinity acceptor tag which can be used to immunopurify the HLA-protein. Suitable tags are well-known in the art. In some embodiments, an affinity acceptor tag is poly-histidine tag, poly-histidine-glycine tag, poly-arginine tag, poly-aspartate tag, poly-cysteine tag, poly-phenylalanine, c-myc tag, Herpes simplex virus glycoprotein D (gD) tag, FLAG tag, KT3 epitope tag, tubulin epitope tag, T7 gene 10 protein peptide tag, streptavidin tag, streptavidin binding peptide (SPB) tag, Strep-tag, Strep-tag II, albumin-binding protein (ABP) tag, alkaline phosphatase (AP) tag, bluetongue virus tag (B-tag), calmodulin binding peptide (CBP) tag, chloramphenicol acetyl transferase (CAT) tag, choline-binding domain (CBD) tag, chitin binding domain (CBD) tag, cellulose binding domain (CBP) tag, dihydrofolate reductase (DHFR) tag, galactose-binding protein (GBP) tag, maltose binding protein (MBP), glutathione-S-transferase (GST), Glu-Glu (EE) tag, human influenza hemagglutinin (HA) tag, horseradish peroxidase (HRP) tag, NE-tag, HSV tag, ketosteroid isomerase (KSI) tag, KT3 tag, LacZ tag, luciferase tag, NusA tag, PDZ domain tag, AviTag, Calmodulin-tag, E-tag, S-tag, SBP-tag, Softag 1, Softag 3, TC tag, VSV-tag, Xpress tag, Isopeptag, SpyTag, SnoopTag, Profinity eXact tag, Protein C tag, S1-tag, S-tag, biotin-carboxy carrier protein (BCCP) tag, green fluorescent protein (GFP) tag, small ubiquitin-like modifier (SUMO) tag, tandem affinity purification (TAP) tag, HaloTag, Nus-tag, Thioredoxin-tag, Fc-tag, CYD tag, HPC tag, TrpE tag, ubiquitin tag, a VSV-G epitope tag derived from the Vescular Stomatis viral glycoprotein, or a V5 tag derived from a small epitope (Pk) found on the P and V proteins of the paramyxovirus of simian virus 5 (SV5). In some embodiments, the affinity acceptor tag is an “epitope tag,” which is a type of peptide tag that adds a recognizable epitope (antibody binding site) to the HLA-protein to provide binding of corresponding antibody, thereby allowing identification or affinity purification of the tagged protein. Non-limiting example of an epitope tag is protein A or protein G, which binds to IgG. In some embodiments, affinity acceptor tags include the biotin acceptor peptide (BAP) or Human influenza hemagglutinin (HA) peptide sequence. Numerous other tag moieties are known to, and can be envisioned by, the ordinarily skilled artisan, and are contemplated herein. Any peptide tag can be used as long as it is capable of being expressed as an element of an affinity acceptor tagged HLA-peptide complex.

The methods provided herein comprise isolating HLA-peptide complexes from the cells transfected or transduced with affinity pulldown of HLA constructs. In some embodiments, the complexes can be isolated using standard immunoprecipitation techniques known in the art with commercially available antibodies. The cells can be first lysed. HLA class I-peptide complexes can be isolated using HLA class I specific antibodies such as the W6/32 antibody, while HLA class II-peptide complexes can be isolated using HLA class II specific antibodies such as the M5/114.15.2 monoclonal antibody. In some embodiments, the single (or pair of) HLA alleles are expressed as a fusion protein with a peptide tag and the HLA-peptide complexes are isolated using binding molecules that recognize the peptide tags.

The methods further comprise isolating peptides from said HLA-peptide complexes and sequencing the peptides. The peptides are isolated from the complex by any method known to one of skill in the art, such as acid elution. While any sequencing method can be used, methods employing mass spectrometry, such as liquid chromatography-mass spectrometry (LC-MS or LC-MS/MS, or alternatively HPLC-MS or HPLC-MS/MS) are utilized in some embodiments.

These sequencing methods are well-known to a skilled person and are reviewed in Medzihradszky KF and Chalkley RJ. Mass Spectrom Rev. 2015 Jan-Feb;34(1):43-63.

In some embodiments, the population of cells expresses one or more endogenous HLA alleles. In some embodiments, the population of cells is an engineered population of cells lacking one or more endogenous HLA class I alleles. In some embodiments, the population of cells is an engineered population of cells lacking endogenous HLA class I alleles. In some embodiments, the population of cells is an engineered population of cells lacking one or more endogenous HLA class II alleles. In some embodiments, the population of cells is an engineered population of cells lacking endogenous HLA class II alleles or an engineered population of cells lacking endogenous HLA class I alleles and endogenous HLA class II alleles. In some embodiments, the population of cells comprises cells that have been enriched or sorted, such as by fluorescence activated cell sorting (FACS). In some embodiments, fluorescence activated cell sorting (FACS) is used to sort the population of cells. In some embodiments, the population of cells is previously FACS sorted for cell surface expression of either HLA class I or class II or both HLA class I and class II. For example, FACS can be used to sort the population of cells for cell surface expression of an HLA class I allele, an HLA class II allele, or a combination thereof.

### Methods for Preparing a Personalized Cancer Vaccine

Once a mutation specific for a cancer is identified, such that the mutation exists in the DNA in cancer cells but not in the normal cells of the same human subject, and the mutation leads to a change in one or more amino acids in the protein encoded by the DNA, the mutation can be a target for the host immune response. A natural immune response can be directed against the mutated protein leading to the destruction of cancer cells expressing the protein. Because of the natural tolerance response and immunocompromised environment in the cancerous tissue, immunotherapy is a clinical path that attempts augmenting such immune response to override the body's tolerance and immunosuppressive effects. A protein or a peptide comprising the mutation as described above is therefore a suitable candidate for immunotherapy.

A mutated protein is ingested by professional phagocytes acting as antigen presenting cells (APCs), chopped and displayed as antigens on the cell surface for T cell activation in an antigen presentation complex comprising a Major Histocompatibility Complex (MHC) protein. Human MHC proteins are called Human Leukocytic antigens, HLAs. The MHC protein can be a MHC-class I or a class II protein, and while several functional distinctions are attributed to the presentation of peptides by either class I or class II MHC proteins (HLA class I and HLA class II proteins), one salient distinction lies in the fact that HLA class I-peptide complexes present antigens to cytotoxic CD8+ T cells, whereas the HLA class II peptide complexes are also capable of activating CD4+ T cell leading to prolonged immune response. CD8+ T cells are indispensable in the task of cell-by-cell elimination of a diseased cell, such as an infected cell or a tumor cell. CD4+ T cells have a more sustained effects upon activation, the most important of those being generation of immunological memory. CD4 subsets are differentially recruited according to the type of immunologic threat, and multiple subsets with overlapping or disparate functions may be co-recruited. This helps in balancing the immunological response with respect to the pathogenic threat. In these respects, HLA class I or class II peptide mediated antigen presentation effects a sustained and tailored immune response. On the other hand, HLA class I or class II binding to peptides may be promiscuous and therefore non-specific peptide binding and presentation to the immune system leads to aberrant immune response, such as autoimmunity.

In one aspect, the present disclosure provides method for predicting peptides that can accurately pair with, or bind to, a specific HLA class I or class II molecule, such that the high fidelity binding of the peptide to HLA class I or class II protein ensures presentation of the specific peptide to the T lymphocytes, thereby eliciting a specific immune response and avoid any cross-reactivity or immune promiscuity.

In one aspect, the present disclosure provides method for predicting peptides that can accurately bind to a specific HLA class I or class II protein, such that a more sustained and robust immune response can be activated with the peptide, when the peptide is administered therapeutically to a subject expressing the specific cognate HLA class I or class II protein, by dint of the ability of HLA class I or class II protein's activation of CD4+ T cells and stimulate immunological memory. In some embodiments, the given peptide that is predicted to bind to a HLA class I or class II protein with high specificity is a peptide comprising a mutation, wherein the mutation is prevalent in a cancer or a tumor cell of a subject; whereas the same HLA class I or class II protein predicted to bind the mutated peptide either (a) does not bind, or (b) binds with distinctly lower affinity to the corresponding non-mutated wild type peptide compared to the affinity for binding to the mutated peptide of the subject. The preferential binding of the HLA to the mutated peptide is advantageous in the development of an immunotherapeutic, since the cells expressing the wild type peptide will be spared from the immune attack by the T cells reactive to the HLA-presented peptide. In some embodiments, predicted peptides that bind specifically to the HLA class I or class II proteins are peptides that have post-translation modifications. Exemplary post-translational modifications include but are not limited to: phosphorylation, ubiquitylation, dephosphorylation, glycosylation, methylation, or, acetylation. In some embodiments, the predicted peptides are subjected to post-translational modifications prior for use in immunotherapy.

In some embodiments, the immunotherapy methods and strategies disclosed herein could also be applicable in suppressing unwanted immune activation, such as, in an autoimmune reaction. Specifically, peptides identified as potential binders for specific HLA subtypes could be tailored to bind to the specific HLA molecule and induces tolerance rather than cause immunogenic response.

In one aspect, presented herein are methods of immunotherapy tailored or personalized for a specific subject. Every subject or patient expresses a specific array of HLA class I and HLA class II proteins. HLA typing is a well-known technique that allows determination of the specific repertoire of HLA proteins expressed by the subject. Once the HLA heterodimers expressed by a specific subject is known, having an improved, sophisticated and reliable method as described herein for predicting peptides that can bind to a specific HLA class I or class II complex, with high fidelity can ensure that a specific immune response can be generated tailored specifically for the subject.

The genes coding for HLA heterodimers are highly polymorphic, with more 4,000 HLA class II allele variants identified across the human population. From maternal and paternal HLA haplotypes, an individual can inherit different alleles for each of the HLA class II loci, and each HLA class II heterodimer is made of an α—and β-chain. Because of the large number of α—and β-chain pairing combinations, especially for HLA-DP and HLA-DQ alleles, the population of possible HLA heterodimers is highly complex. HLA class II heterodimers are translated in the endoplasmic reticulum (ER) and assembled into a stable complex with the invariant chain (II) derived from the protein CD74. The II stabilizes the class II complex by allowing proper protein folding and enables the export of HLA class II heterodimers into endosomal/lysosomal compartments. Inside these HLA class II loading compartments, the II is proteolytically cleaved by cathepsins into a placeholder peptide called CLIP. CLIP is then exchanged for higher-affinity peptides in a low pH environment by the chaperone HLA-DM, a non-classical HLA class II heterodimer. High affinity peptide-loaded HLA class II complexes are then to the trans-Golgi and finally to the cell surface for display for CD4+ T cells.

Each HLA heterodimer is estimated to bind thousands of peptides with allele-specific binding preferences. In fact, each HLA allele is estimated to bind and present ˜1,000-10,000 unique peptides to T cells. Given such diversity in HLA binding, accurate prediction of whether a peptide is likely to bind to a specific HLA allele is highly challenging. Less is known about allele-specific peptide-binding characteristics of HLA class II molecules because of the heterogeneity of α—and β-chain pairing, complexity of data limiting the ability to confidently assign core binding epitopes, and the lack of immunoprecipitation grade, allele-specific antibodies required for high-resolution biochemical analyses. Furthermore, analyzing peptide epitopes derived from a given HLA allele raises ambiguity when multiple HLA alleles are presented on a cell surface.

Disclosed herein are methods to preparing a personalized cancer vaccine. The method for preparing a personalized cancer vaccine may comprise identifying peptide sequences with a mutation expressed in cancer cells of a subject; inputting amino acid position information of the peptide sequences identified, using a computer processor, into a machine-learning HLA-peptide presentation prediction model to generate a set of presentation predictions for the peptide sequences identified, each presentation prediction representing a probability that one or more proteins encoded by a class I or class II MHC allele of a cancer cell of the subject will present a given sequence of a peptide sequence identified; and selecting a subset of the peptide sequences identified based on the set of presentation predictions for preparing the personalized cancer vaccine.

In some embodiments, one or more results obtained from a method described herein may provide a quantitative value or values indicative of one or more of the following: a likelihood of diagnostic accuracy, a likelihood of a presence of a condition in a subject, a likelihood of a subject developing a condition, a likelihood of success of a particular treatment, or any combination thereof. In some embodiments, a method as described herein may predict a risk or likelihood of developing a condition. In some embodiments, a method as described herein may be an early diagnostic indicator of developing a condition. In some embodiments, a method as described herein may confirm a diagnosis or a presence of a condition. In some embodiments, a method as described herein may monitor the progression of a condition. In some embodiments, a method as described herein may monitor the efficacy of a treatment for a condition in a subject.

### Method for Identification of MHC Presenting Peptides

In one aspect, presented herein is a method of identifying one or more peptides that are presented by MHC proteins for immune activation. In some embodiments, the one r more peptides comprise an epitope. In some embodiments, the method involves computational prediction of the likelihood that specific epitopes are presented by an MHC protein. In some embodiments, the method involves computational prediction of the specificity of an epitope for MHC presentation. In some embodiments, the computational prediction methods involve an assessment of peptide-MHC interactions. In some embodiments, the computational prediction methods involve an prediction of the allelic specificity of a peptide for antigen presentation.

In some embodiments, the computational prediction methods involve integration of bioinformatics information, for example, nucleotide sequences, structural motifs of biomolecules, protein-protein interaction features and functional potency such as immunogenicity. In some embodiments, the computational prediction methods involve machine learning. Many immunoinformatics methods for prediction of peptide-MHC interactions have been developed for both MHC class I and II, based on machine learning approaches such as simple pattern motif, support vector machine (SVM), hidden Markov model (HMM), neural network (NN) models, quantitative structure-activity relationship (QSAR) analysis, structure-based methods, and biophysical methods. These methods can be divided into two categories, namely, intra-allele (allele-specific) and trans-allele (pan-specific) methods. Intra-allelic methods are trained for a specific MHC molecule on a limited set of experimental peptide-binding data and applied for prediction of peptides binding to that molecule. Because of the extreme polymorphism of MHC molecules, the existence of thousands of allele variants, combined with the lack of sufficient experimental binding data, it is impossible to build a prediction model for each allele. Thus, trans-allele and general purpose methods such as NetMHCIIpan (Karosiene E etal., NetMHCIIpan-3.0, a common pan-specific MHC class II prediction method including all three human MHC class II isotypes, HLA-DR, HLA-DP and HLADQ. Immunogenetics (2013) 65(10):711-24), and TEPITOPEpan (Zhang L, et al., TEPITOPEpan: extending TEPITOPE for peptide binding prediction covering over 700 HLA-DR molecules. PLoS One (2012) 7(2):e30483) have been developed using peptide-binding data expanding over many alleles or across species. Similar methods for MHC-I are also available such as NetMHCpan and KISS.

In some embodiments, the peptide sequences may not be expressed in normal cells of the subject. In some embodiments, each and every cell of the subject may not be cancer cells. The cancer cells may be produced through different cancers, including, but not limited to, thyroid cancer, adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's disease, cervical cancer, childhood Non-Hodgkin's lymphoma, lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, uterine cancer (e.g. uterine sarcoma), vaginal cancer, vulvar cancer, or Waldenstrom's macroglobulinemia.

The identifying may comprise comparing DNA, RNA or protein sequences from the cancer cells of the subject to DNA, RNA or protein sequences from the normal cells of the subject. The DNA, RNA or protein sequences from the cancer cells of the subject may be different from the DNA, RNA or protein sequences from the normal cells of the subject. The identifying may identify nucleic acid variants with high sensitivity.

The machine-learning HLA-peptide presentation prediction model may comprise a plurality of predictor variables identified at least based on training data. The training data may comprises sequence information of sequences of peptides presented by an HLA protein expressed in cells and identified by mass spectrometry; training peptide sequence information comprising amino acid position information, wherein the training peptide sequence information is associated with the HLA protein expressed in cells; and a function representing a relation between the amino acid position information received as input and the presentation likelihood generated as output based on the amino acid position information and the predictor variables.

In some embodiments, the training data may further comprise structured data, time-series data, unstructured data, and relational data. Unstructured data may comprise audio data, image data, video, mechanical data, electrical data, chemical data, and any combination thereof, for use in accurately simulating or training robotics or simulations. Time-series data may comprise data from one or more of a smart meter, a smart appliance, a smart device, a monitoring system, a telemetry device, or a sensor. Relational data comprises data from a customer system, an enterprise system, an operational system, a website, web accessible application program interface (API), or any combination thereof. This may be done by a user through any method of inputting files or other data formats into software or systems.

In some embodiments, the training data may be stored in a database. A database can be stored in computer readable format. A computer processor may be configured to access the data stored in the computer readable memory. In some embodiments, the computer system may be used to analyze the data to obtain a result. The result may be stored remotely or internally on storage medium, and communicated to personnel such as medication professionals. In some embodiments, the computer system may be operatively coupled with components for transmitting the result. Components for transmitting can include wired and wireless components. Examples of wired communication components can include a Universal Serial Bus (USB) connection, a coaxial cable connection, an Ethernet cable such as a Cat5 or Cat6 cable, a fiber optic cable, or a telephone line. Examples or wireless communication components can include a Wi-Fi receiver, a component for accessing a mobile data standard such as a 3G or 4G LTE data signal, or a Bluetooth receiver. In some embodiments, all these data in the storage medium is collected and archived to build a data warehouse.

In some embodiments, the database comprises an external database. The external database may be a medical database, for example, but not limited to, Adverse Drug Effects Database, AHFS Supplemental File, Allergen Picklist File, Average WAC Pricing File, Brand Probability File, Canadian Drug File v2, Comprehensive Price History, Controlled Substances File, Drug Allergy Cross-Reference File, Drug Application File, Drug Dosing & Administration Database, Drug Image Database v2.0/Drug Imprint Database v2.0, Drug Inactive Date File, Drug Indications Database, Drug Lab Conflict Database, Drug Therapy Monitoring System (DTMS) v2.2/DTMS Consumer Monographs, Duplicate Therapy Database, Federal Government Pricing File, Healthcare Common Procedure Coding System Codes (HCPCS) Database, ICD-10 Mapping Files, Immunization Cross-Reference File, Integrated A to Z Drug Facts Module, Integrated Patient Education, Master Parameters Database, Medi-Span Electronic Drug File (MED-File) v2, Medicaid Rebate File, Medicare Plans File, Medical Condition Picklist File, Medical Conditions Master Database, Medication Order Management Database (MOMD), Parameters to Monitor Database, Patient Safety Programs File, Payment Allowance Limit-Part B (PAL-B) v2.0, Precautions Database, RxNorm Cross-Reference File, Standard Drug Identifiers Database, Substitution Groups File, Supplemental Names File, Uniform System of Classification Cross-Reference File, or Warning Label Database.

In some embodiments, the training data may also be obtained through other data sources. The data sources may include sensors or smart devices, such as appliances, smart meters, wearables, monitoring systems, data stores, customer systems, billing systems, financial systems, crowd source data, weather data, social networks, or any other sensor, enterprise system or data store. Example of smart meters or sensors may include meters or sensors located at a customer site, or meters or sensors located between customers and a generation or source location. By incorporating data from a broad array of sources, the system may be capable of performing complex and detailed analyses. In some embodiments, the data sources may include sensors or databases for other medical platforms without limitation.

HLA-typing is conventionally carried out by either serological methods using antibodies or by PCR-based methods such as Sequence Specific Oligonucleotide Probe Hybridization (SSOP), or Sequence Based Typing (SBT). While the first is hampered by the potentially high degree of cross reactivity and limited resolution capabilities, the second suffers from difficulties associated with the efficiency of the PCR due to very limited possibilities for positioning primers because of polymorphic positions.

In some embodiments, the sequence information is identified by either sequencing methods or methods employing mass spectrometry, such as liquid chromatography-mass spectrometry (LC-MS or LC-MS/MS, or alternatively HPLC-MS or HPLC-MS/MS). These sequencing methods may be well-known to a skilled person and are reviewed in Medzihradszky KF and Chalkley RJ. Mass Spectrom Rev. 2015 Jan-Feb;34(1):43-63. In some embodiments, the mass spectrometry is mono-allelic mass spectrometry. In some embodiments, the mass spectrometry may be MS analysis, MS/MS analysis, LC-MS/MS analysis, or a combination thereof. In some embodiments, MS analysis may be used to determine a mass of an intact peptide. For example, the determining can comprise determining a mass of an intact peptide (e.g., MS analysis). In some embodiments, MS/MS analysis may be used to determine a mass of peptide fragments. For example, the determining can comprise determining a mass of peptide fragments, which can be used to determine an amino acid sequence of a peptide or portion thereof (e.g., MS/MS analysis). In some embodiments, the mass of peptide fragments may be used to determine a sequence of amino acids within the peptide. In some embodiments, LC-MS/MS analysis may be used to separate complex peptide mixtures. For example, the determining can comprise separating complex peptide mixtures, such as by liquid chromatography, and determining a mass of an intact peptide, a mass of peptide fragments, or a combination thereof (e.g., LC-MS/MS analysis). This data can be used, e.g., for peptide sequencing.

In some embodiments, the training peptide sequence information comprises amino acid position information of training peptides. In some embodiments, the training peptide sequence information comprises at most about 90%, 80%, 70%, 60%, 50%, 40%, 30%, 20%, 10% or less sequence information of sequences of peptides presented by an HLA protein expressed in cells and identified by mass spectrometry. In some embodiments, the training peptide sequence information may comprise at least about 10%, 20%, 30%, 40%, 50%, 60%, 70%, 80%, 90% or more sequence information of sequences of peptides presented by an HLA protein expressed in cells and identified by mass spectrometry.

Any information and data may be paired with a subject who is the source of the information and data. The subject or medical professional can retrieve the information and data from a storage or a server through a subject identity. A subject identity may comprise patient's photo, name, address, social security number, birthday, telephone number, zip code, or any combination thereof. A subject identity may be encrypted and encoded in a visual graphical code. A visual graphical code may be a one-time barcode that can be uniquely associated with a subject identity. A barcode may be a UPC barcode, EAN barcode, Code 39 barcode, Code 128 barcode, ITF barcode, CodaBar barcode, GS1 DataBar barcode, MSI Plessey barcode, QR barcode, Datamatrix code, PDF417 code, or an Aztec barcode. A visual graphical code may be configured to be displayed on a display screen. A barcode may comprise QR that can be optically captured and read by a machine. A barcode may define an element such as a version, format, position, alignment, or timing of the barcode to enable reading and decoding of the barcode. A barcode can encode various types of information in any type of suitable format, such as binary or alphanumeric information. A QR code can have various symbol sizes as long as the QR code can be scanned from a reasonable distance by an imaging device. A QR code can be of any image file format (e.g. EPS or SVG vector graphs, PNG, TIF, GIF, or JPEG raster graphics format).

In some embodiments, the function representing a relation between the amino acid position information received as input and the presentation likelihood generated as output based on the amino acid position information and the predictor variables comprises a linear or non-linear function. The function may be, for example, a rectified linear unit (ReLU) activation function, a Leaky ReLu activation function, or other function such as a saturating hyperbolic tangent, identity, binary step, logistic, arcTan, softsign, parameteric rectified linear unit, exponential linear unit, softPlus, bent identity, softExponential, Sinusoid, Sinc, Gaussian, or sigmoid function, or any combination thereof.

In some embodiments, the linear function is obtained through linear regression. In some embodiments, the linear regression is a method to predict a target variable by fitting the best linear relationship between the dependent and independent variable. The best fit may mean that the sum of all the distances between the shape and the actual observations at each point is the least. Linear regression may comprise simple linear regression or multiple linear regression. The simple linear regression may use a single independent variable to predict a dependent variable. The multiple linear regressions may use more than one independent variables to predict a dependent variable by fitting a best linear relationship. The non-linear function may be obtained through non-linear regression. The nonlinear regression may be a form of regression analysis in which observational data are modeled by a function which is a nonlinear combination of the model parameters and depends on one or more independent variables. The nonlinear regression may comprise a step function, piecewise function, spline, and generalized additive model.

In some embodiments, the presentation likelihood is presented by one-dimensional values (e.g., probabilities). In some embodiments, the probability is configured to measure the likelihood that an event may occur. In some embodiments, the probability ranges from about 0 and 1, 0.1 to 0.9, 0.2 to 0.8, 0.3 to 0.7, or 0.4 to 0.6. The higher the probability of an event, the more likely the event may occur. In some embodiments, the event comprises any type of situation, including, by way of non-limiting examples, whether the HLA-peptide will present some peptide with certain amino acid position information, and whether a person will be sick based on amino acid position information. In some embodiments, the likelihood may be presented by multi-dimensional values. The multi-dimensional values may be presented by multi-dimensional space, heatmap, or spreadsheet.

In one embodiment, selecting a subset of the peptide sequences identified based on the set of presentation predictions is configured to prepare the personalized cancer vaccine. In some embodiments, the subset comprises at most about 90%, 80%, 70%, 60%, 50%, 40%, 30%, 20%, 10% or less of the peptide sequences identified based on the set of presentation predictions. In other cases, the subset may comprise at least about 10%, 20%, 30%, 40%, 50%, 60%, 70%, 80%, 90% or more of the peptide sequences identified based on the set of presentation predictions. A cancer vaccine may be a vaccine that either treats existing cancer or prevents development of a cancer. Vaccines may be prepared from samples taken from the patient, and may be specific to that patient.

In some embodiments, a Poxvirus is used in the disease (e.g., cancer) vaccine or immunogenic composition. These include orthopoxvirus, avipox, vaccinia, MVA, NYVAC, canarypox, ALVAC, fowlpox, TROVAC, etc. Advantages of the vectors may include simple construction, ability to accommodate large amounts of foreign DNA and high expression levels. Information concerning poxviruses that can be used in the practice of the disclosure, such as Chordopoxvirinae subfamily poxviruses (poxviruses of vertebrates), for instance, orthopoxviruses and avipoxviruses, e.g., vaccinia virus (e.g., Wyeth Strain, WR Strain (e.g., ATCC® VR-1354), Copenhagen Strain, NYVAC, NYVAC.1, NYVAC.2, MVA, MVA-BN), canarypox virus (e.g., Wheatley C93 Strain, ALVAC), fowlpox virus (e.g., FP9 Strain, Webster Strain, TROVAC), dovepox, pigeonpox, quailpox, and raccoon pox, inter alia, synthetic or non-naturally occurring recombinants thereof, uses thereof, and methods for making and using such recombinants can be found in scientific and patent literature.

In some embodiments, a vaccinia virus is used in the disease vaccine or immunogenic composition to express an antigen. The recombinant vaccinia virus may be able to replicate within the cytoplasm of the infected host cell and the polypeptide of interest may therefore induce an immune response.

In some embodiments, ALVAC is used as a vector in a disease vaccine or immunogenic composition. ALVAC may be a canarypox virus that can be modified to express foreign transgenes and has been used as a method for vaccination against both prokaryotic and eukaryotic antigens.

In some embodiments, a Modified Vaccinia Ankara (MVA) virus is used as a viral vector for an antigen vaccine or immunogenic composition. MVA may be a member of the Orthopoxvirus family and has been generated by about 570 serial passages on chicken embryo fibroblasts of the Ankara strain of Vaccinia virus (CVA). As a consequence of these passages, the resulting MVA virus may comprise 31 kilobases fewer genomic information compared to CVA, and is highly host-cell restricted. MVA may be characterized by its extreme attenuation, namely, by a diminished virulence or infectious ability, but still holds an excellent immunogenicity. When tested in a variety of animal models, MVA may be proven to be avirulent, even in immuno-suppressed individuals. Moreover, MVA-BN®-HER2 may be a candidate immunotherapy designed for the treatment of HER-2-positive breast cancer and is currently in clinical trials.

In some embodiments, a positive predictive value (PPV) is used as part of the prediction model. A PPV, also known as a precision measurement, is the probability that an individual diagnosed with a disease or condition through, for example, a test or model, actually has the disease or condition. It can be calculated by dividing the number of true positive results by the total number of results that returned positive (results that include false positives). PPV=True Positives/(True positives+False positives). For example, if in a set of 100 patients, the model identified a positive result in 50 patients, of which 25 were true positives, the PPV would be 25/50=0.5. A PPV closer to 1 represents a more accurate diagnosis method, such as a test or model. A PPV may be used to determine the accuracy of the prediction model. A PPV may be used to adjust the prediction model to accommodate for false positive results that may be generated by the model.

A recall rate may be used as part of the prediction model. A recall rate may be considered as the percentage of true positive results out of the total number of positives in the sample set. Recall=True Positives/(True positives+False Negatives). For example, if in a set of 100 patients, the model identified a positive result in 50 patients, of which 25 were true positives, and there were a total of 75 positives in the set of patients, the recall rate would be {25/(25+25)}×100=50%. A recall rate may be used to determine the accuracy of the prediction model. A recall rate may be used to adjust the prediction model to accommodate for false positive results or false negative results that may be generated by the model.

In some embodiments, the prediction model has a positive predictive value of at least 0.05, 0.1, 0.2, 0.25, 0.3, 0.4, 0.5, 0.6, 0.7, 0.8, 0.9 or greater at a recall rate of from 0.1%-10%. In some embodiments, the prediction model may have a positive predictive value of at most 0.9, 0.8, 0.7, 0.6, 0.5, 0.4, 0.3, 0.2, 0.1 or less at a recall rate of from 0.1%-10%. The prediction model may have a positive predictive value of at least 0.05, 0.1, 0.2, 0.3, 0.4, 0.5, 0.6, 0.7, 0.8, 0.9 or greater at a recall rate less than 0.1%. In some embodiments, the prediction model may have a positive predictive value of at most 0.9, 0.8, 0.7, 0.6, 0.5, 0.4, 0.3, 0.2, 0.1 or less at a recall rate less than 0.1%. The prediction model may have a positive predictive value of at least 0.05, 0.1, 0.2, 0.3, 0.4, 0.5, 0.6, 0.7, 0.8, 0.9 or greater at a recall rate more than 10%. In some embodiments, the prediction model may have a positive predictive value of at most 0.9, 0.8, 0.7, 0.6, 0.5, 0.4, 0.3, 0.2, 0.1 or less at a recall rate more than 10%.

In some embodiments, the prediction model has a positive predictive value of at least 0.05, 0.1, 0.2, 0.25, 0.3, 0.4, 0.5, 0.6, 0.7, 0.8, 0.9 or greater at a recall rate of 0.1% to 10%. In some embodiments, the prediction model has a positive predictive value of at least 0.05, 0.1, 0.2, 0.25, 0.3, 0.4, 0.5, 0.6, 0.7, 0.8, 0.9 or greater at a recall rate of 0.10% to 0.50%, 0.10% to 10%, 0.10% to 2%, 0.10% to 30%, 0.10% to 4%, 0.10% to 50%, 0.10% to 6%, 0.10% to 70%, 0.10% to 80%, 0.10% to 9%, 0.10% to 10%, 0.5% to 1%, 0.5% to 2%, 0.5% to 3%, 0.5% to 4%, 0.5% to 5%, 0.5% to 6%, 0.5% to 7%, 0.5% to 8%, 0.5% to 9%, 0.5% to 10%, 1% to 2%, 1% to 3%, 1% to 4%, 1% to 5%, 1% to 6%, 1% to 7%, 1% to 8%, 1% to 9%, 1% to 10%, 2% to 3%, 2% to 4%, 2% to 5%, 2% to 6%, 2% to 7%, 2% to 8%, 2% to 9%, 2% to 10%, 3% to 4%, 3% to 5%, 3% to 6%, 3% to 7%, 3% to 8%, 3% to 9%, 3% to 10%, 4% to 5%, 4% to 6%, 4% to 7%, 4% to 8%, 4% to 9%, 4% to 10%, 5% to 6%, 5% to 7%, 5% to 8%, 5% to 9%, 5% to 10%, 6% to 7%, 6% to 8%, 6% to 9%, 6% to 10%, 7% to 8%, 7% to 9%, 7% to 10%, 8% to 9%, 8% to 10%, or 9% to 10%. In some embodiments, the prediction model has a positive predictive value of at least 0.05, 0.1, 0.2, 0.25, 0.3, 0.4, 0.5, 0.6, 0.7, 0.8, 0.9 or greater at a recall rate of 0.1%, 0.5%, 1%, 2%, 3%, 4%, 5%, 6%, 7%, 8%, 9%, or 10%. In some embodiments, the prediction model has a positive predictive value of at least 0.05, 0.1, 0.2, 0.25, 0.3, 0.4, 0.5, 0.6, 0.7, 0.8, 0.9 or greater at a recall rate of at least 0.1%, 0.5%, 1%, 2%, 3%, 4%, 5%, 6%, 7%, 8%, or 9%. In some embodiments, the prediction model has a positive predictive value of at least 0.05, 0.1, 0.2, 0.25, 0.3, 0.4, 0.5, 0.6, 0.7, 0.8, 0.9 or greater at a recall rate of at most 0.5%, 1%, 2%, 3%, 4%, 5%, 6%, 7%, 8%, 9%, or 10%.

In some embodiments, the prediction model has a positive predictive value of at least 0.05, 0.1, 0.2, 0.25, 0.3, 0.4, 0.5, 0.6, 0.7, 0.8, 0.9 or greater at a recall rate of 10% to 20%. In some embodiments, the prediction model has a positive predictive value of at least 0.05, 0.1, 0.2, 0.25, 0.3, 0.4, 0.5, 0.6, 0.7, 0.8, 0.9 or greater at a recall rate of 10% to 11%, 10% to 12%, 10% to 13%, 10% to 14%, 10% to 15%, 10% to 16%, 10% to 17%, 10% to 18%, 10% to 19%, 10% to 20%, 11% to 12%, 11% to 13%, 11% to 14%, 11% to 15%, 11% to 16%, 11% to 17%, 11% to 18%, 11% to 19%, 11% to 20%, 12% to 13%, 12% to 14%, 12% to 15%, 12% to 16%, 12% to 17%, 12% to 18%, 12% to 19%, 12% to 20%, 13% to 14%, 13% to 15%, 13% to 16%, 13% to 17%, 13% to 18%, 13% to 19%, 13% to 20%, 14% to 15%, 14% to 16%, 14% to 17%, 14% to 18%, 14% to 19%, 14% to 20%, 15% to 16%, 15% to 17%, 15% to 18%, 15% to 19%, 15% to 20%, 16% to 17%, 16% to 18%, 16% to 19%, 16% to 20%, 17% to 18%, 17% to 19%, 17% to 20%, 18% to 19%, 18% to 20%, or 19% to 20%. In some embodiments, the prediction model has a positive predictive value of at least 0.05, 0.1, 0.2, 0.25, 0.3, 0.4, 0.5, 0.6, 0.7, 0.8, 0.9 or greater at a recall rate of 10%, 11%, 12%, 13%, 14%, 15%, 16%, 17%, 18%, 19%, or 20%. In some embodiments, the prediction model has a positive predictive value of at least 0.05, 0.1, 0.2, 0.25, 0.3, 0.4, 0.5, 0.6, 0.7, 0.8, 0.9 or greater at a recall rate of at least 10%, 11%, 12%, 13%, 14%, 15%, 16%, 17%, 18%, or 19%. In some embodiments, the prediction model has a positive predictive value of at least 0.05, 0.1, 0.2, 0.25, 0.3, 0.4, 0.5, 0.6, 0.7, 0.8, 0.9 or greater at a recall rate of at most 11%, 12%, 13%, 14%, 15%, 16%, 17%, 18%, 19%, or 2⁰%.

In some embodiments, the prediction model has a positive predictive value of at least 0.05, 0.1, 0.2, 0.25, 0.3, 0.4, 0.5, 0.6, 0.7, 0.8, 0.9 or greater at a recall rate of at least 0.1%, 0.5%, 1%, 2%, 3%, 4%, 5%, 6%, 7%, 8%, 9%10%1, 1%1, 2%1, 3%1, 14%, 15%, 6%1, 7%1, 8%1, 19%, or 20%. For example, prediction model may have a positive predictive value of at least 0.1 at a recall rate of at least 10%. For example, prediction model may have a positive predictive value of at least 0.2 at a recall rate of at least 10%. For example, prediction model may have a positive predictive value of at least 0.3 at a recall rate of at least 10%. For example, prediction model may have a positive predictive value of at least 0.4 at a recall rate of at least 10%. For example, prediction model may have a positive predictive value of at least 0.5 at a recall rate of at least 10%. For example, prediction model may have a positive predictive value of at least 0.6 at a recall rate of at least 10%. For example, prediction model may have a positive predictive value of at least 0.7 at a recall rate of at least 10%. For example, prediction model may have a positive predictive value of at least 0.8 at a recall rate of at least 10%. For example, prediction model may have a positive predictive value of at least 0.9 at a recall rate of at least 10%. For example, prediction model may have a positive predictive value of at least 0.1 at a recall rate of at least 5%. For example, prediction model may have a positive predictive value of at least 0.2 at a recall rate of at least 5%. For example, prediction model may have a positive predictive value of at least 0.3 at a recall rate of at least 5%. For example, prediction model may have a positive predictive value of at least 0.4 at a recall rate of at least 5%. For example, prediction model may have a positive predictive value of at least 0.5 at a recall rate of at least 5%. For example, prediction model may have a positive predictive value of at least 0.6 at a recall rate of at least 5%. For example, prediction model may have a positive predictive value of at least 0.7 at a recall rate of at least 5%. For example, prediction model may have a positive predictive value of at least 0.8 at a recall rate of at least 5%. For example, prediction model may have a positive predictive value of at least 0.9 at a recall rate of at least 5%. For example, prediction model may have a positive predictive value of at least 0.1 at a recall rate of at least 20%. For example, prediction model may have a positive predictive value of at least 0.2 at a recall rate of at least 20%. For example, prediction model may have a positive predictive value of at least 0.3 at a recall rate of at least 20%. For example, prediction model may have a positive predictive value of at least 0.4 at a recall rate of at least 20%. For example, prediction model may have a positive predictive value of at least 0.5 at a recall rate of at least 20%. For example, prediction model may have a positive predictive value of at least 0.6 at a recall rate of at least 20%. For example, prediction model may have a positive predictive value of at least 0.7 at a recall rate of at least 20%. For example, prediction model may have a positive predictive value of at least 0.8 at a recall rate of at least 20%. For example, prediction model may have a positive predictive value of at least 0.9 at a recall rate of at least 20%.

In some embodiments, the prediction model has a positive predictive value of at least 0.05, 0.1, 0.2, 0.25, 0.3, 0.4, 0.5, 0.6, 0.7, 0.8, 0.9 or greater at a recall rate of about 0.1%, 0.5%, 1%, 2%, 3%, 4%, 5%, 6%, 7%, 8%, 9%10%1, 1%1, 2%1, 3%1, 14%, 15%, 6%1, 7%1, 8%1, 19%, or 20%. For example, prediction model may have a positive predictive value of at least 0.1 at a recall rate of about 10%. For example, prediction model may have a positive predictive value of at least 0.2 at a recall rate of about 10%. For example, prediction model may have a positive predictive value of at least 0.3 at a recall rate of about 10%. For example, prediction model may have a positive predictive value of at least 0.4 at a recall rate of about 10%. For example, prediction model may have a positive predictive value of at least 0.5 at a recall rate of about 10%. For example, prediction model may have a positive predictive value of at least 0.6 at a recall rate of about 10%. For example, prediction model may have a positive predictive value of at least 0.7 at a recall rate of about 10%. For example, prediction model may have a positive predictive value of at least 0.8 at a recall rate of about 10%. For example, prediction model may have a positive predictive value of at least 0.9 at a recall rate of about 10%. For example, prediction model may have a positive predictive value of at least 0.1 at a recall rate of about 5%. For example, prediction model may have a positive predictive value of at least 0.2 at a recall rate of about 5%. For example, prediction model may have a positive predictive value of at least 0.3 at a recall rate of about 5%. For example, prediction model may have a positive predictive value of at least 0.4 at a recall rate of about 5%. For example, prediction model may have a positive predictive value of at least 0.5 at a recall rate of about 5%. For example, prediction model may have a positive predictive value of at least 0.6 at a recall rate of about 5%. For example, prediction model may have a positive predictive value of at least 0.7 at a recall rate of about 5%. For example, prediction model may have a positive predictive value of at least 0.8 at a recall rate of about 5%. For example, prediction model may have a positive predictive value of at least 0.9 at a recall rate of about 5%. For example, prediction model may have a positive predictive value of at least 0.1 at a recall rate of about 20%. For example, prediction model may have a positive predictive value of at least 0.2 at a recall rate of about 20%. For example, prediction model may have a positive predictive value of at least 0.3 at a recall rate of about 20%. For example, prediction model may have a positive predictive value of at least 0.4 at a recall rate of about 20%. For example, prediction model may have a positive predictive value of at least 0.5 at a recall rate of about 20%. For example, prediction model may have a positive predictive value of at least 0.6 at a recall rate of about 20%. For example, prediction model may have a positive predictive value of at least 0.7 at a recall rate of about 20%. For example, prediction model may have a positive predictive value of at least 0.8 at a recall rate of about 20%. For example, prediction model may have a positive predictive value of at least 0.9 at a recall rate of about 20%.

In some embodiments, the prediction model has a positive predictive value of at least 0.05, 0.1, 0.2, 0.25, 0.3, 0.4, 0.5, 0.6, 0.7, 0.8, 0.9 or greater at a recall rate of less than 0.1%, 0.5%, 1%, 2%, 3%, 4%, 5%, 6%, 7%, 8%, 9%10%1, 1%1, 2%1, 3%1, 14%, 15%, 6%1, 7%1, 8%1, 19%, or 20%. For example, prediction model may have a positive predictive value of at least 0.1 at a recall rate of at most 10%. For example, prediction model may have a positive predictive value of at least 0.2 at a recall rate of at most 10%. For example, prediction model may have a positive predictive value of at least 0.3 at a recall rate of at most 10%. For example, prediction model may have a positive predictive value of at least 0.4 at a recall rate of at most 10%. For example, prediction model may have a positive predictive value of at least 0.5 at a recall rate of at most 10%. For example, prediction model may have a positive predictive value of at least 0.6 at a recall rate of at most 10%. For example, prediction model may have a positive predictive value of at least 0.7 at a recall rate of at most 10%. For example, prediction model may have a positive predictive value of at least 0.8 at a recall rate of at most 10%. For example, prediction model may have a positive predictive value of at least 0.9 at a recall rate of at most 10%. For example, prediction model may have a positive predictive value of at least 0.1 at a recall rate of at most 5%. For example, prediction model may have a positive predictive value of at least 0.2 at a recall rate of at most 5%. For example, prediction model may have a positive predictive value of at least 0.3 at a recall rate of at most 5%. For example, prediction model may have a positive predictive value of at least 0.4 at a recall rate of at most 5%. For example, prediction model may have a positive predictive value of at least 0.5 at a recall rate of at most 5%. For example, prediction model may have a positive predictive value of at least 0.6 at a recall rate of at most 5%. For example, prediction model may have a positive predictive value of at least 0.7 at a recall rate of at most 5%. For example, prediction model may have a positive predictive value of at least 0.8 at a recall rate of at most 5%. For example, prediction model may have a positive predictive value of at least 0.9 at a recall rate of at most 5%. For example, prediction model may have a positive predictive value of at least 0.1 at a recall rate of at most 20%. For example, prediction model may have a positive predictive value of at least 0.2 at a recall rate of at most 20%. For example, prediction model may have a positive predictive value of at least 0.3 at a recall rate of at most 20%. For example, prediction model may have a positive predictive value of at least 0.4 at a recall rate of at most 20%. For example, prediction model may have a positive predictive value of at least 0.5 at a recall rate of at most 20%. For example, prediction model may have a positive predictive value of at least 0.6 at a recall rate of at most 20%. For example, prediction model may have a positive predictive value of at least 0.7 at a recall rate of at most 20%. For example, prediction model may have a positive predictive value of at least 0.8 at a recall rate of at most 20%. For example, prediction model may have a positive predictive value of at least 0.9 at a recall rate of at most 20%.

In some embodiments, at a recall rate of about 0.1%, 0.5%, 1%, 2%, 3%, 4%, 5%, 6%, 7%, 8%, 9%, 10%, 11%, 12%, 13%, 14%, 15%, 16%, 17%, 18%, 19%, or 20% the prediction model has a positive predictive value of 0.05% to 0.6%. At a recall rate of about 0.1%, 0.5%, 1%, 2%, 3%, 4%, 5%, 6%, 7%, 8%, 9%, 10%, 11%, 12%, 13%, 14%, 15%, 16%, 17%, 18%, 19%, or 20% the prediction model may have a positive predictive value of 0.05% to 0.1%, 0.05% to 0.15%, 0.05% to 0.2%, 0.05% to 0.25%, 0.05% to 0.3%, 0.05% to 0.35%, 0.05% to 0.4%, 0.05% to 0.45%, 0.05% to 0.5%, 0.05% to 0.55%, 0.05% to 0.6%, 0.1% to 0.15%, 0.1% to 0.2%, 0.1% to 0.25%, 0.1% to 0.3%, 0.1% to 0.35%, 0.1% to 0.4%, 0.1% to 0.45%, 0.1% to 0.5%, 0.1% to 0.55%, 0.1% to 0.6%, 0.15% to 0.2%, 0.15% to 0.25%, 0.15% to 0.3%, 0.15% to 0.35%, 0.15% to 0.4%, 0.15% to 0.45%, 0.15% to 0.5%, 0.15% to 0.55%, 0.15% to 0.6%, 0.2% to 0.25%, 0.2% to 0.3%, 0.2% to 0.35%, 0.2% to 0.4%, 0.2% to 0.45%, 0.2% to 0.5%, 0.2% to 0.55%, 0.2% to 0.6%, 0.25% to 0.3%, 0.25% to 0.35%, 0.25% to 0.4%, 0.25% to 0.45%, 0.25% to 0.5%, 0.25% to 0.55%, 0.25% to 0.6%, 0.3% to 0.35%, 0.3% to 0.4%, 0.3% to 0.45%, 0.3% to 0.5%, 0.3% to 0.55%, 0.3% to 0.6%, 0.35% to 0.4%, 0.35% to 0.45%, 0.35% to 0.5%, 0.35% to 0.55%, 0.35% to 0.6%, 0.4% to 0.45%, 0.4% to 0.5%, 0.4% to 0.55%, 0.4% to 0.6%, 0.45% to 0.5%, 0.45% to 0.55%, 0.45% to 0.6%, 0.5% to 0.55%, 0.5% to 0.6%, or 0.55% to 0.6%. At a recall rate of about 0.1%, 0.5%, 1%, 2%, 3%, 4%, 5%, 6%, 7%, 8%, 9%, 10%, 11%, 12%, 13%, 14%, 1%, %¹⁶%, 17%, 18%, 19%, or 20% the prediction model may have a positive predictive value of 0.05%, 0.1%, 0.15%, 0.2%, 0.25%, 0.3%, 0.35%, 0.4%, 0.45%, 0.5%, 0.55%, or 0.6%. At a recall rate of about 0.1%, 0.5%, 1%, 2%, 3%, 4%, 5%, 6%, 7%, 8%, 9%, 10%, 11%, 12%, 13%, 14%, 15%, 16%, 17%, 18%, 19%, or 20% the prediction model may have a positive predictive value of at least 0.05%, 0.1%, 0.15%, 0.2%, 0.25%, 0.3%, 0.35%, 0.4%, 0.45%, 0.5%, or 0.55%. At a recall rate of about 0.1%, 0.5%, 1%, 2%, 3%, 4%, 5%, 6%, 7%, 8%, 9%, 10%, 11%, 12%, 13%, 14%, 15%, 16%, 17%, 18%, 19%, or 20% the prediction model may have a positive predictive value of at most 0.1%, 0.15%, 0.2%, 0.25%, 0.3%, 0.35%, 0.4%, 0.45%, 0.5%, 0.55%, or 0.6%.

At a recall rate of about 0.1%, 0.5%, 1%, 2%, 3%, 4%, 5%, 6%, 7%, 8%, 9%, 10%, 11%, 12%, 13%, 14%, 15%, 16%, 17%, 18%, 19%, or 20% the prediction model may have a positive predictive value of 0.45% to 0.98%. At a recall rate of about 0.1%, 0.5%, 1%, 2%, 3%, 4%, 5%, 6%, 7%, 8%, 9%,10%1, 11%, 12%, 13%, 14%, 15%, 16%, 17%, 18%, 19%, or 20% the prediction model may have a positive predictive value of 0.45% to 0.5%, 0.45% to 0.55%, 0.45% to 0.6%, 0.45% to 0.65%, 0.45% to 0.7%, 0.45% to 0.75%, 0.45% to 0.8%, 0.45% to 0.85%, 0.45% to 0.9%, 0.45% to 0.96%, 0.45% to 0.98%, 0.5% to 0.55%, 0.5% to 0.6%, 0.5% to 0.65%, 0.5% to 0.7%, 0.5% to 0.75%, 0.5% to 0.8%, 0.5% to 0.85%, 0.5% to 0.9%, 0.5% to 0.96%, 0.5% to 0.98%, 0.55% to 0.6%, 0.55% to 0.65%, 0.55% to 0.7%, 0.55% to 0.75%, 0.55% to 0.8%, 0.55% to 0.85%, 0.55% to 0.9%, 0.55% to 0.96%, 0.55% to 0.98%, 0.6% to 0.65%, 0.6% to 0.7%, 0.6% to 0.75%, 0.6% to 0.8%, 0.6% to 0.85%, 0.6% to 0.9%, 0.6% to 0.96%, 0.6% to 0.98%, 0.65% to 0.7%, 0.65% to 0.75%, 0.65% to 0.8%, 0.65% to 0.85%, 0.65% to 0.9%, 0.65% to 0.96%, 0.65% to 0.98%, 0.7% to 0.75%, 0.7% to 0.8%, 0.7% to 0.85%, 0.7% to 0.9%, 0.7% to 0.96%, 0.7% to 0.98%, 0.75% to 0.8%, 0.75% to 0.85%, 0.75% to 0.9%, 0.75% to 0.96%, 0.75% to 0.98%, 0.8% to 0.85%, 0.8% to 0.9%, 0.8% to 0.96%, 0.8% to 0.98%, 0.85% to 0.9%, 0.85% to 0.96%, 0.85% to 0.98%, 0.9% to 0.96%, 0.9% to 0.98%, or 0.96% to 0.98%. At a recall rate of about 0.1%, 0.5%, 1%, 2%, 3%, 4%, 5%, 6%, 7%, 8%, 9%, 10, 1%, 12%, 13%, 14%, 15%, 16%, 17%, 18%, 19%, or 20% the prediction model may have a positive predictive value of 0.45%, 0.5%, 0.55%, 0.6%, 0.65%, 0.7%, 0.75%, 0.8%, 0.85%, 0.9%, 0.96%, or 0.98%. At a recall rate of about 0.1%, 0.5%, 1%, 2%, 3%, 4%, 5%, 6%, 7%, 8%, 9%, 10, 1%, 12%, 13%, 14%, 15%, 16%, 17%, 18%, 19%, or 20% the prediction model may have a positive predictive value of at least 0.45%, 0.5%, 0.55%, 0.6%, 0.65%, 0.7%, 0.75%, 0.8%, 0.85%, 0.9%, or 0.96%. At a recall rate of about 0.1%, 0.5%, 1%, 2%, 3%, 4%, 5%, 6%, 7%, 8%, 9%, 10, 1%, 12%, 13%, 14%, 15%, 16%, 17%, 18%, 19%, or 20% the prediction model may have a positive predictive value of at most 0.5%, 0.55%, 0.6%, 0.65%, 0.7%, 0.75%, 0.8%, 0.85%, 0.9%, 0.96%, or 0.98%.

### Methods of Training a Machine-Learning HLA-Peptide Presentation Prediction Model

In an aspect, a method of training a machine-learning HLA-peptide presentation prediction model may comprise inputting amino acid position information sequences of HLA-peptides isolated from one or more HLA-peptide complexes from a cell expressing an HLA class I or class II allele into the HLA-peptide presentation prediction model using a computer processor; training the machine-learning HLA-peptide presentation prediction model may comprise adjusting weighted values on nodes of a neural network to best match the provided training data.

The training data may comprise sequence information of sequences of peptides presented by an HLA protein expressed in cells and identified by mass spectrometry; training peptide sequence information comprising amino acid position information of training peptides, wherein the training peptide sequence information is associated with the HLA protein expressed in cells; and a function representing a relation between the amino acid position information received as input and a presentation likelihood generated as output based on the amino acid position information and the predictor variables. The training data, training peptide sequence information, function, and presentation likelihood are disclosed elsewhere herein.

The trained algorithm may comprise one or more neural networks. A neural network may be a type of computing system based upon a graph of several connected neurons (or nodes) in a series of layers. A neural network may comprise an input layer, to which data is presented; one or more internal, and/or “hidden,” layers; and an output layer, from which results are presented. A neural network may learn the relationships between an input data set and a target data set by adjusting a series of connection weights. A neuron may be connected to neurons in other layers via connections that have weights, which are parameters that control the strength of a connection. The number of neurons in each layer may be related to the complexity of a problem to be solved. The minimum number of neurons required in a layer may be determined by the problem complexity, and the maximum number may be limited by the ability of a neural network to generalize. Input neurons may receive data being presented and then transmit that data to a node in the first hidden layer through connection weights, which are modified during training. The result node may sum up the products of all pairs of inputs and their associated weights. The weighted sum may be offset with a bias to adjust the value of the result node. The output of a node or neuron may be gated using a threshold or activation function. An activation function may be a linear or non-linear function. An activation function may be, for example, a rectified linear unit (ReLU) activation function, a Leaky ReLu activation function, or other function such as a saturating hyperbolic tangent, identity, binary step, logistic, arcTan, softsign, parameteric rectified linear unit, exponential linear unit, softPlus, bent identity, softExponential, Sinusoid, Sinc, Gaussian, or sigmoid function, or any combination thereof.

A hidden layer in the neural network may process data and transmit its result to the next layer through a second set of weighted connections. Each subsequent layer may “pool” results from previous layers into more complex relationships. Neural networks may be trained with a known sample set of training data (data collected from one or more sensors) by allowing them to modify themselves during (and after) training so as to provide a desired output from a given set of inputs, such as an output value. A trained algorithm may comprise convolutional neural networks, recurrent neural networks, dilated convolutional neural networks, fully connected neural networks, deep generative models, and Boltzmann machines.

Weighing factors, bias values, and threshold values, or other computational parameters of a neural network, may be “taught” or “learned” in a training phase using one or more sets of training data. For example, parameters may be trained using input data from a training data set and a gradient descent or backward propagation method so that output value(s) from a neural network are consistent with examples included in a training data set.

The number of nodes used in an input layer of a neural network may be at least about 10, 50, 100, 200, 300, 400, 500, 600, 700, 800, 900, 1000, 2000, 3000, 4000, 5000, 6000, 7000, 8000, 9000, 10,000, 20,000, 30,000, 40,000, 50,000, 60,000, 70,000, 80,000, 90,000, 100,000 or greater.

In other instances, the number of node used in an input layer may be at most about 100,000, 90,000, 80,000, 70,000, 60,000, 50,000, 40,000, 30,000, 20,000, 10,000, 9000, 8000, 7000, 6000, 5000, 4000, 3000, 2000, 1000, 900, 800, 700, 600, 500, 400, 300, 200, 100, 50, or 10 or smaller. In some instance, the total number of layers used in a neural network (including input and output layers) may be at least about 3, 4, 5, 10, 15, 20, or greater. In other instances, the total number of layers may be at most about 20, 15, 10, 5, 4, 3 or less.

In some instances, the total number of learnable or trainable parameters, e.g., weighting factors, biases, or threshold values, used in a neural network may be at least about 10, 50, 100, 200, 300, 400, 500, 600, 700, 800, 900, 1000, 2000, 3000, 4000, 5000, 6000, 7000, 8000, 9000, 10,000, 20,000, 30,000, 40,000, 50,000, 60,000, 70,000, 80,000, 90,000, 100,000 or greater. In other instances, the number of learnable parameters may be at most about 100,000, 90,000, 80,000, 70,000, 60,000, 50,000, 40,000, 30,000, 20,000, 10,000, 9000, 8000, 7000, 6000, 5000, 4000, 3000, 2000, 1000, 900, 800, 700, 600, 500, 400, 300, 200, 100, 50, or 10 or smaller.

A neural network may comprise a convolutional neural network. A convolutional neural network may comprise one or more convolutional layers, dilated layers or fully connected layers. The number of convolutional layers may be between 1-10 and dilated layers between 0-10. The total number of convolutional layers (including input and output layers) may be at least about 1,2, 3, 4, 5, 10, 15, 20, or greater, and the total number of dilated layers may be at least about 1,2, 3, 4, 5, 10, 15, 20, or greater. The total number of convolutional layers may be at most about 20, 15, 10, 5, 4, 3 or less, and the total number of dilated layers may be at most about 20, 15, 10, 5, 4, 3 or less. In some embodiments, the number of convolutional layers is between 1-10 and fully connected layers between 0-10. The total number of convolutional layers (including input and output layers) may be at least about 1,2, 3, 4, 5, 10, 15, 20, or greater, and the total number of fully connected layers may be at least about 1,2, 3, 4, 5, 10, 15, 20, or greater. The total number of convolutional layers may be at most about 20, 15, 10, 5, 4, 3 or less, and the total number of fully connected layers may be at most about 20, 15, 10, 5, 4, 3 or less.

A convolutional neural network (CNN) may be a deep and feed-forward artificial neural network. A CNN may be applicable to analyzing visual imagery. A CNN may comprise an input, an output layer, and multiple hidden layers. Hidden layers of a CNN may comprise convolutional layers, pooling layers, fully connected layers and normalization layers. Layers may be organized in 3 dimensions: width, height and depth.

Convolutional layers may apply a convolution operation to an input and pass results of a convolution operation to a next layer. For processing images, a convolution operation may reduce the number of free parameters, allowing a network to be deeper with fewer parameters. In a convolutional layer, neurons may receive input from only a restricted subarea of a previous layer. Convolutional layer's parameters may comprise a set of learnable filters (or kernels). Learnable filters may have a small receptive field and extend through the full depth of an input volume. During a forward pass, each filter may be convolved across the width and height of an input volume, compute a dot product between entries of a filter and an input, and produce a 2-dimensional activation map of that filter. As a result, a network may learn filters that activate when it detects some specific type of feature at some spatial position in an input.

Pooling layers may comprise global pooling layers. Global pooling layers may combine outputs of neuron clusters at one layer into a single neuron in the next layer. For example, max pooling layers may use the maximum value from each of a cluster of neurons at a prior layer; and average pooling layers may use an average value from each of a cluster of neurons at the prior layer. Fully connected layers may connect every neuron in one layer to every neuron in another layer. In a fully-connected layer, each neuron may receive input from every element of a previous layer. A normalization layer may be a batch normalization layer. A batch normalization layer may improve performance and stability of neural networks. A batch normalization layer may provide any layer in a neural network with inputs that are zero mean/unit variance. Advantages of using batch normalization layer may include faster trained networks, higher learning rates, easier to initialize weights, more activation functions viable, and simpler process of creating deep networks.

A neural network may comprise a recurrent neural network. A recurrent neural network may be configured to receive sequential data as an input, such as consecutive data inputs, and a recurrent neural network software module may update an internal state at every time step. A recurrent neural network can use internal state (memory) to process sequences of inputs. A recurrent neural network may be applicable to tasks such as handwriting recognition or speech recognition, next word prediction, music composition, image captioning, time series anomaly detection, machine translation, scene labeling, and stock market prediction. A recurrent neural network may comprise fully recurrent neural network, independently recurrent neural network, Elman networks, Jordan networks, Echo state, neural history compressor, long short-term memory, gated recurrent unit, multiple timescales model, neural Turing machines, differentiable neural computer, neural network pushdown automata, or any combination thereof.

A trained algorithm may comprise a supervised or unsupervised learning method such as, for example, SVM, random forests, clustering algorithm (or software module), gradient boosting, logistic regression, and/or decision trees. Supervised learning algorithms may be algorithms that rely on the use of a set of labeled, paired training data examples to infer the relationship between an input data and output data. Unsupervised learning algorithms may be algorithms used to draw inferences from training data sets to output data. Unsupervised learning algorithms may comprise cluster analysis, which may be used for exploratory data analysis to find hidden patterns or groupings in process data. One example of an unsupervised learning method may comprise principal component analysis. Principal component analysis may comprise reducing the dimensionality of one or more variables. The dimensionality of a given variables may be at least 1, 5, 10, 50, 100, 200, 300, 400, 500, 600, 700, 800, 900, 1000, 1100, 1200 1300, 1400, 1500, 1600, 1700, 1800, or greater. The dimensionality of a given variables may be at most 1800, 1600, 1500, 1400, 1300, 1200, 1100, 1000, 900, 800, 700, 600, 500, 400, 300, 200, 100, 50, 10 or less.

A training algorithm may be obtained through statistical techniques. In some embodiments, statistical techniques may comprise linear regression, classification, resampling methods, subset selection, shrinkage, dimension reduction, nonlinear models, tree-based methods, support vector machines, unsupervised learning, or any combination thereof.

A linear regression may be a method to predict a target variable by fitting the best linear relationship between a dependent and independent variable. The best fit may mean that the sum of all distances between a shape and actual observations at each point is the least. Linear regression may comprise simple linear regression and multiple linear regression. A simple linear regression may use a single independent variable to predict a dependent variable. A multiple linear regression may use more than one independent variable to predict a dependent variable by fitting a best linear relationship.

A classification may be a data mining technique that assigns categories to a collection of data in order to achieve accurate predictions and analysis. Classification techniques may comprise logistic regression and discriminant analysis. Logistic regression may be used when a dependent variable is dichotomous (binary). Logistic regression may be used to discover and describe a relationship between one dependent binary variable and one or more nominal, ordinal, interval or ratio-level independent variables. A resampling may be a method comprising drawing repeated samples from original data samples. A resampling may not involve a utilization of a generic distribution tables in order to compute approximate probability values. A resampling may generate a unique sampling distribution on a basis of an actual data. In some embodiments, a resampling may use experimental methods, rather than analytical methods, to generate a unique sampling distribution. Resampling techniques may comprise bootstrapping and cross-validation. Bootstrapping may be performed by sampling with replacement from original data, and take “not chosen” data points as test cases. Cross validation may be performed by split training data into a plurality of parts.

A subset selection may identify a subset of predictors related to a response. A subset selection may comprise best-subset selection, forward stepwise selection, backward stepwise selection, hybrid method, or any combination thereof. In some embodiments, shrinkage fits a model involving all predictors, but estimated coefficients are shrunken towards zero relative to the least squares estimates. This shrinkage may reduce variance. A shrinkage may comprise ridge regression and a lasso. A dimension reduction may reduce a problem of estimating n+1 coefficients to a simpler problem of m+1 coefficients, where m<n. It may be attained by computing n different linear combinations, or projections, of variables. Then these n projections are used as predictors to fit a linear regression model by least squares. Dimension reduction may comprise principal component regression and partial least squares. A principal component regression may be used to derive a low-dimensional set of features from a large set of variables. A principal component used in a principal component regression may capture the most variance in data using linear combinations of data in subsequently orthogonal directions. The partial least squares may be a supervised alternative to principal component regression because partial least squares may make use of a response variable in order to identify new features.

A nonlinear regression may be a form of regression analysis in which observational data are modeled by a function which is a nonlinear combination of model parameters and depends on one or more independent variables. A nonlinear regression may comprise a step function, piecewise function, spline, generalized additive model, or any combination thereof.

Tree-based methods may be used for both regression and classification problems. Regression and classification problems may involve stratifying or segmenting the predictor space into a number of simple regions. Tree-based methods may comprise bagging, boosting, random forest, or any combination thereof. Bagging may decrease a variance of prediction by generating additional data for training from the original dataset using combinations with repetitions to produce multistep of the same camality/size as original data. Boosting may calculate an output using several different models and then average a result using a weighted average approach. A random forest algorithm may draw random bootstrap samples of a training set. Support vector machines may be classification techniques. Support vector machines may comprise finding a hyperplane that best separates two classes of points with the maximum margin. Support vector machines may constrain an optimization problem such that a margin is maximized subject to a constraint that it perfectly classifies data.

Unsupervised methods may be methods to draw inferences from datasets comprising input data without labeled responses. Unsupervised methods may comprise clustering, principal component analysis, k-Mean clustering, hierarchical clustering, or any combination thereof.

The mass spectrometry may be mono-allelic mass spectrometry. In some embodiments, the mass spectrometry may be MS analysis, MS/MS analysis, LC-MS/MS analysis, or a combination thereof. In some embodiments, MS analysis may be used to determine a mass of an intact peptide. For example, the determining can comprise determining a mass of an intact peptide (e.g., MS analysis). In some embodiments, MS/MS analysis may be used to determine a mass of peptide fragments. For example, the determining can comprise determining a mass of peptide fragments, which can be used to determine an amino acid sequence of a peptide or portion thereof (e.g., MS/MS analysis). In some embodiments, the mass of peptide fragments may be used to determine a sequence of amino acids within the peptide. In some embodiments, LC-MS/MS analysis may be used to separate complex peptide mixtures. For example, the determining can comprise separating complex peptide mixtures, such as by liquid chromatography, and determining a mass of an intact peptide, a mass of peptide fragments, or a combination thereof (e.g., LC-MS/MS analysis). This data can be used, e.g., for peptide sequencing.

The peptides may be presented by an HLA protein expressed in cells through autophagy. Autophagy may allow the orderly degradation and recycling of cellular components. The autophagy may comprise macroautophagy, microautophagy and Chaperone mediated autophagy. The peptides may be presented by an HLA protein expressed in cells through phagocytosis. The phagocytosis may be a major mechanism used to remove pathogens and cell debris. For example, when a macrophage ingests a pathogenic microorganism, the pathogen becomes trapped in a phagosome which then fuses with a lysosome to form a phagolysosome. In HLA class II, phagocytes such as macrophages and immature dendritic cells may take up entities by phagocytosis into phagosomes—though B cells exhibit the more general endocytosis into endosomes—which fuse with lysosomes whose acidic enzymes cleave the uptaken protein into many different peptides.

The quality of the training data may be increased by using a plurality of quality metrics. The plurality of quality metrics may comprise common contaminant peptide removal, high scored peak intensity, high score, and high mass accuracy. The scored peak intensity may be used prior to performing scoring. The MS/MS Search first screens the MS/MS spectrum against candidate sequences using a simple filter. This filter may be minimum scored peak intensity. Using the scored peak intensity may enhance search speed by allowing candidate sequences to be rapidly and summarily rejected once a sufficient number of spectral peaks are examined and found not to meet the threshold established by this filter. The scored peak intensity may be at least 50%. The scored peak intensity may be at least 70%. The scored peak intensity may be at least 10%, 20%, 30%, 40%, 50%, 60%, 70%, 80%, 90% or greater. In some cases, the scored peak intensity may be at most 90%, 80%, 70%, 60%, 50%, 40%, 30%, 20%, 10% or less. The score may be at least 7. The score may be at least 1, 2, 3, 4, 5, 6, 7, 8, 9, 10, 15, 20 or greater. In some cases, the score may be at most about 20, 15, 10, 9, 8, 7, 6, 5, 4, 3, 2, 1 or less. The mass accuracy may be at most 5 ppm. The mass accuracy may be at most 10 ppm, 9 ppm, 8 ppm, 7 ppm, 6 ppm, 5 ppm, 4 ppm, 3 ppm, 2 ppm, 1 ppm or less. The mass accuracy may be at least 1 ppm, 2 ppm, 3 ppm, 4 ppm, 5 ppm, 6 ppm, 7 ppm, 8 ppm, 9 ppm, 10 ppm or greater.

In some embodiments, a mass accuracy is at most 2 ppm. In some embodiments, a backbone cleavage score is at least 5. In some embodiments, a backbone cleavage score is at least 8.

The peptides presented by an HLA protein expressed in cells may be peptides presented by a single immunoprecipitated HLA protein expressed in cells. Immunoprecipitation (IP) may be the technique of precipitating a protein antigen out of solution using an antibody that specifically binds to that particular protein. This process can be used to isolate and concentrate a particular protein from a sample containing many thousands of different proteins. Immunoprecipitation may require that the antibody be coupled to a solid substrate at some point in the procedure.

The peptides presented by an HLA protein expressed in cells may be peptides presented by a single exogenous HLA protein expressed in cells. The single exogenous HLA protein may be created by introducing one or more exogenous peptides to the population of cells. In some embodiments, the introducing comprises contacting the population of cells with the one or more exogenous peptides or expressing the one or more exogenous peptides in the population of cells. In some embodiments, the introducing comprises contacting the population of cells with one or more nucleic acids encoding the one or more exogenous peptides. In some embodiments, the one or more nucleic acids encoding the one or more peptides is DNA. In some embodiments, the one or more nucleic acids encoding the one or more peptides is RNA, optionally wherein the RNA is mRNA. In some embodiments, the enriching does not comprise use of a tetramer (or multimer) reagent.

The peptides presented by an HLA protein expressed in cells may be peptides presented by a single recombinant HLA protein expressed in cells. The recombinant HLA protein may be encoded by a recombinant HLA class I or HLA class II allele. The HLA class I may be selected from the group consisting of HLA-A, HLA-B, HLA-C. The HLA class I may be a non-classical class-I-b group. The HLA class I may be selected from the group consisting of HLA-E, HLA-F, and HLA-G. The HLA class I may be a non-classical class-I-b group selected from the group consisting of HLA-E, HLA-F, and HLA-G. In some embodiments, the HLA class II comprises an HLA class II α-chain, an HLA class II β-chain, or a combination thereof.

The plurality of predictor variables may comprise a peptide-HLA affinity predictor variable. The plurality of predictor variables may comprise a source protein expression level predictor variable. The source protein expression level may be the expression level of the source protein of the peptide within a cell. In some embodiments, the expression level may be determined by measuring the amount of source protein or the amount of RNA encoding the source protein.

The plurality of predictor variables may comprise peptide sequence, amino acid physical properties, peptide physical properties, expression level of the source protein of a peptide within a cell, protein stability, protein translation rate, ubiquitination sites, protein degradation rate, translational efficiencies from ribosomal profiling, protein cleavability, protein localization, motifs of host protein that facilitate TAP transport, host protein is subject to autophagy, motifs that favor ribosomal stalling (e.g., polyproline or polylysine stretches), protein features that favor NMD (e.g., long 3′ UTR, stop codon >50nt upstream of last exon:exon junction and peptide cleavability).

The plurality of predictor variables may comprise a peptide cleavability predictor variable. The peptide cleavability may be associated with a cleavable linker or a cleavage sequence. In some embodiments, the cleavable linker is a ribosomal skipping site or an internal ribosomal entry site (IRES) element. In some embodiments, the ribosomal skipping site or IRES is cleaved when expressed in the cells. In some embodiments, the ribosomal skipping site is selected from the group consisting of F2A, T2A, P2A, and E2A. In some embodiments, the IRES element is selected from common cellular or viral IRES sequences. A cleavage sequence, such as F2A, or an internal ribosome entry site (IRES) can be placed between the α-chain and β2-microglobulin (HLA class I) or between the α-chain and β-chain (HLA class II). In some embodiments, a single HLA class I allele is HLA-A*02:01, HLA-A*23:01 and HLA-B*14:02, or HLA-E*01:01, and HLA class II allele is HLA-DRB*01:01, HLA-DRB*01:02 and HLA-DRB*11:01, HLA-DRB*15:01, or HLA-DRB*07:01. In some embodiments, the cleavage sequence is a T2A, P2A, E2A, or F2A sequence. For example, the cleavage sequence can be E G R G S L T C G D V E N P G P (SEQ ID NO: 6) (T2A), A T N F S L K Q A G D V E N P G P (SEQ ID NO: 7) (P2A), Q C T N Y A L K L A G D V E S N P G P (SEQ ID NO: 8) (E2A), or V K Q T L N F D L K L A G D V E S N P G P (SEQ ID NO: 9) (F2A).

In some embodiments, the cleavage sequence may be a thrombin cleavage site CLIP.

The peptides presented by the HLA protein may comprise peptides that are identified by searching a no-enzyme specificity without modification peptide database. The peptide database may be a no-enzyme specificity peptide database, such as a without modification database or a with modification (e.g., phosphorylation or cysteinylation) database. In some embodiments, the peptide database is a polypeptide database. In some embodiments, the polypeptide database may be a protein database. In some embodiments, the method further comprises searching the peptide database using a reversed-database search strategy. In some embodiments, the method further comprises searching a protein database using a reversed-database search strategy. In some embodiments, a de novo search is performed, e.g., to discover new peptides that are not included in a normal peptide or protein database. The peptide database may be generated by providing a first and a second population of cells each comprising one or more cells comprising an affinity acceptor tagged HLA, wherein the sequence affinity acceptor tagged HLA comprises a different recombinant polypeptide encoded by a different HLA allele operatively linked to an affinity acceptor peptide; enriching for affinity acceptor tagged HLA-peptide complexes; characterizing a peptide or a portion thereof bound to an affinity acceptor tagged HLA-peptide complex from the enriching; and generating an HLA-allele specific peptide database.

The peptides presented by the HLA protein may comprise peptides identified by comparing a MS/MS spectra of the HLA-peptides with MS/MS spectra of one or more HLA-peptides in a peptide database.

There may be mutation on either peptides or nucleic acid that encodes peptides. The mutation may be selected from the group consisting of a point mutation, a splice site mutation, a frameshift mutation, a read-through mutation, and a gene fusion mutation. The point mutation may be a genetic mutation where a single nucleotide base is changed, inserted or deleted from a sequence of DNA or RNA. The splice site mutation may be a genetic mutation that inserts, deletes or changes a number of nucleotides in the specific site at which splicing takes place during the processing of precursor messenger RNA into mature messenger RNA. The frameshift mutation may be a genetic mutation caused by indels (insertions or deletions) of a number of nucleotides in a DNA sequence that is not divisible by three. The mutation may also comprise insertions, deletions, substitution mutations, gene duplications, chromosomal translocations, and chromosomal inversions.

In some embodiments, the HLA class II protein comprises an HLA-DR protein.

In some embodiments, the HLA class II protein comprises an HLA-DP protein.

In some embodiments, the HLA class II protein comprises an HLA-DQ protein.

In some embodiments, the HLA class II protein may be selected from the group consisting of an HLA-DR, and HLA-DP or an HLA-DQ protein. In some embodiments, the HLA protein is an HLA class II protein selected from the group consisting of: HLA-DPB1*01:01/HLA-DPA1*01:03, HLA-DPB1*02:01/HLA-DPA1*01:03, HLA-DPB1*03:01/HLA-DPA1*01:03, HLA-DPB1*04:01/HLA-DPA1*01:03, HLA-DPB1*04:02/HLA-DPA1*01:03, HLA-DPB1*06:01/HLA-DPA1*01:03, HLA-DQB1*02:01/HLA-DQA1*05:01, HLA-DQB1*02:02/HLA-DQA1*02:01, HLA-DQB1*06:02/HLA-DQA1*01:02, HLA-DQB1*06:04/HLA-DQA1*01:02, HLA-DRB1*01:01, HLA-DRB1*01:02, HLA-DRB1*03:01, HLA-DRB1*03:02, HLA-DRB1*04:01, HLA-DRB1*04:02, HLA-DRB1*04:03, HLA-DRB1*04:04, HLA-DRB1*04:05, HLA-DRB1*04:07, HLA-DRB1*07:01, HLA-DRB1*08:01, HLA-DRB1*08:02, HLA-DRB1*08:03, HLA-DRB1*08:04, HLA-DRB1*09:01, HLA-DRB1*10:01, HLA-DRB1*11:01, HLA-DRB1*11:02, HLA-DRB1*11:04, HLA-DRB1*12:01, HLA-DRB1*12:02, HLA-DRB1*13:01, HLA-DRB1*13:02, HLA-DRB1*13:03, HLA-DRB1*14:01, HLA-DRB1*15:01, HLA-DRB1*15:02, HLA-DRB1*15:03, HLA-DRB1*16:01, HLA-DRB3*01:01, HLA-DRB3*02:02, HLA-DRB3*03:01, HLA-DRB4*01:01, HLA-DRB5*01:01). The peptides presented by the HLA protein may have a length of from 15-40 amino acids. The peptides presented by the HLA protein may have a length of at least 1, 2, 3, 4, 5, 6, 7, 8, 9, 10, 11, 12, 13, 14, 15, 16, 17, 18, 19, 20, 21, 22, 23, 24, 25, 26, 27, or greater amino acids. In some embodiments, the peptides presented by the HLA protein may have a length of at most 30, 29, 28, 27, 26, 25, 24, 23, 22, 21, 20, 19, 18, 17, 16, 15, 14, 13, 12, 11, or less amino acids.

The peptides presented by the HLA protein may comprise peptides identified by (a) isolating one or more HLA complexes from a cell line expressing a single HLA class II allele; (b) isolating one or more HLA-peptides from the one or more isolated HLA complexes; (c) obtaining MS/MS spectra for the one or more isolated HLA-peptides; and (d) obtaining a peptide sequence that corresponds to the MS/MS spectra of the one or more isolated HLA-peptides from a peptide database; wherein one or more sequences obtained from steps (a, b, c) and (d) identifies the sequence of the one or more isolated HLA-peptides.

The isolating may comprise isolating HLA-peptide complexes from the cells transfected or transduced with affinity tagged HLA constructs. In some embodiments, the complexes can be isolated using standard immunoprecipitation techniques known in the art with commercially available antibodies. The cells can be first lysed. HLA class II-peptide complexes can be isolated using HLA class II specific antibodies such as the M5/114.15.2 monoclonal antibody. In some embodiments, the single (or pair of) HLA alleles are expressed as a fusion protein with a peptide tag and the HLA-peptide complexes are isolated using binding molecules that recognize the peptide tags.

The isolating may comprise isolating peptides from the HLA-peptide complexes and sequencing the peptides. The peptides are isolated from the complex by any method known to one of skill in the art, such as acid elution. While any sequencing method can be used, methods employing mass spectrometry, such as liquid chromatography-mass spectrometry (LC-MS or LC-MS/MS, or alternatively HPLC-MS or HPLC-MS/MS) are utilized in some embodiments. These sequencing methods may be well-known to a skilled person and are reviewed in Medzihradszky KF and Chalkley RJ. Mass Spectrom Rev. 2015 Jan-Feb;34(1):43-63.

Additional candidate components and molecules suitable for isolation or purification may comprise binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes. Purification methods such as cation exchange chromatography can be used to separate conjugates by charge difference, which effectively separates conjugates into their various molecular weights. The content of the fractions obtained by cation exchange chromatography can be identified by molecular weight using conventional methods, for example, mass spectroscopy, SDS-PAGE, or other known methods for separating molecular entities by molecular weight.

In some embodiments, the method further comprises isolating peptides from the affinity acceptor tagged HLA-peptide complexes before the characterizing. In some embodiments, an HLA-peptide complex is isolated using an anti-HLA antibody. In some cases, an HLA-peptide complex with or without an affinity tag is isolated using an anti-HLA antibody. In some cases, a soluble HLA (sHLA) with or without an affinity tag is isolated from media of a cell culture. In some cases, a soluble HLA (sHLA) with or without an affinity tag is isolated using an anti-HLA antibody. For example, an HLA, such as a soluble HLA (sHLA) with or without an affinity tag, can be isolated using a bead or column containing an anti-HLA antibody. In some embodiments, the peptides are isolated using anti-HLA antibodies. In some cases, a soluble HLA (sHLA) with or without an affinity tag is isolated using an anti-HLA antibody. In some cases, a soluble HLA (sHLA) with or without an affinity tag is isolated using a column containing an anti-HLA antibody. In some embodiments, the method further comprises removing one or more amino acids from a terminus of a peptide bound to an affinity acceptor tagged HLA-peptide complex.

The personalized cancer vaccine may further comprise an adjuvant. For example, poly-ICLC, an agonist of TLR3 and the RNA helicase-domains of MDA5 and RIG3, has shown several desirable properties for a vaccine adjuvant. These properties may include the induction of local and systemic activation of immune cells in vivo, production of stimulatory chemokines and cytokines, and stimulation of antigen-presentation by DCs. Furthermore, poly-ICLC can induce durable CD4+ and CD8+ responses in humans. Importantly, striking similarities in the upregulation of transcriptional and signal transduction pathways may be seen in subjects vaccinated with poly-ICLC and in volunteers who had received the highly effective, replication-competent yellow fever vaccine. Furthermore, >90% of ovarian carcinoma patients immunized with poly-ICLC in combination with a NYESO-1 peptide vaccine (in addition to Montanide) showed induction of CD4+ and CD8+ T cell, as well as antibody responses to the peptide in a recent phase 1 study.

The personalized cancer vaccine may further comprise an immune checkpoint inhibitor. The immune checkpoint inhibitor may comprise a type of drug that blocks certain proteins made by some types of immune system cells, such as T cells, and some cancer cells. These proteins help keep immune responses in check and can keep T cells from killing cancer cells. When these proteins are blocked, the “brakes” on the immune system are released and T cells are able to kill cancer cells better. Examples of checkpoint proteins found on T cells or cancer cells include PD-1/PD-L1 and CTLA-4/B7-1/B7-2. Some immune checkpoint inhibitors are used to treat cancer.

The training data may further comprise structured data, time-series data, unstructured data, and relational data. Unstructured data may comprise audio data, image data, video, mechanical data, electrical data, chemical data, and any combination thereof, for use in accurately simulating or training robotics or simulations. Time-series data may comprise data from one or more of a smart meter, a smart appliance, a smart device, a monitoring system, a telemetry device, or a sensor. Relational data comprises data from a customer system, an enterprise system, an operational system, a website, web accessible application program interface (API), or any combination thereof. This may be done by a user through any method of inputting files or other data formats into software or systems.

The training data may be uploaded to a cloud-based database. The cloud-based database may be accessible from local and/or remote computer systems on which the machine learning-based sensor signal processing algorithms are running. The cloud-based database and associated software may be used for archiving electronic data, sharing electronic data, and analyzing electronic data. The data or datasets generated locally may be uploaded to a cloud-based database, from which it may be accessed and used to train other machine learning-based detection systems at the same site or a different site. Sensor device and system test results generated locally may be uploaded to a cloud-based database and used to update the training data set in real time for continuous improvement of sensor device and detection system test performance.

The training may be performed using convolutional neural networks. The convolutional neural network (CNN) is described elsewhere herein. The convolutional neural networks may comprise at least two convolutional layers. The number of convolutional layers may be between 1-10 and the dilated layers between 0-10. The total number of convolutional layers (including input and output layers) may be at least about 1,2, 3, 4, 5, 10, 15, 20, or greater, and the total number of dilated layers may be at least about 1,2, 3, 4, 5, 10, 15, 20, or greater. The total number of convolutional layers may be at most about 20, 15, 10, 5, 4, 3 or less, and the total number of dilated layers may be at most about 20, 15, 10, 5, 4, 3 or less. In some embodiments, the number of convolutional layers is between 1-10 and the fully connected layers between 0-10. The total number of convolutional layers (including input and output layers) may be at least about 1,2, 3, 4, 5, 10, 15, 20, or greater, and the total number of fully connected layers may be at least about 1,2, 3, 4, 5, 10, 15, 20, or greater. The total number of convolutional layers may be at most about 20, 15, 10, 5, 4, 3 or less, and the total number of fully connected layers may be at most about 20, 15, 10, 5, 4, 3 or less.

The convolutional neural networks may comprise at least one batch normalization step. The batch normalization layer may improve the performance and stability of neural networks. The batch normalization layer may provide any layer in a neural network with inputs that are zero mean/unit variance. The total number of batch normalization layers may be at least about 3, 4, 5, 10, 15, 20 or more. The total number of batch normalization layers may be at most about 20, 15, 10, 5, 4, 3 or less.

The convolutional neural networks may comprise at least one spatial dropout step. The total number of spatial dropout steps may be at least about 3, 4, 5, 10, 15, 20 or more, and the total number of spatial dropout steps may be at most about 20, 15, 10, 5, 4, 3 or less.

The convolutional neural networks may comprise at least one global max pooling step. The global pooling layers may combine the outputs of neuron clusters at one layer into a single neuron in the next layer. For example, max pooling layers may use the maximum value from each of a cluster of neurons at the prior layer; and average pooling layers may use the average value from each of a cluster of neurons at the prior layer. The convolutional neural networks may comprise at least about 1, 2, 3, 4, 5, 10, 15, 20, or greater global max pooling steps. The convolutional neural networks may comprise at most about 20, 15, 10, 5, 4, 3 or less global max pooling steps.

The convolutional neural networks may comprise at least one dense layer. The convolutional neural networks may comprise at least about 1, 2, 3, 4, 5, 10, 15, 20, or greater dense layers. The convolutional neural networks may comprise at most about 20, 15, 10, 5, 4, 3 or less dense layers.

### Therapeutic Methods

Personalized immunotherapy using tumor-specific peptides has been described. Tumor neoantigens, which arise as a result of genetic change (e.g., inversions, translocations, deletions, missense mutations, splice site mutations, etc.) within malignant cells, represent the most tumor-specific class of antigens. Neoantigens have rarely been used in cancer vaccine or immunogenic compositions due to technical difficulties in identifying them, selecting optimized antigens, and producing neoantigens for use in a vaccine or immunogenic composition. Efficiently choosing which particular peptides to utilize as an immunogen requires the ability to predict which tumor-specific peptides would efficiently bind to the HLA alleles present in a patient and would be effectively presented to the patient's immune system for inducing anti-tumor immunity. One of the critical barriers to developing curative and tumor-specific immunotherapy is the identification and selection of highly specific and restricted tumor antigens to avoid autoimmunity. This is particularly important in case of candidate tumor specific peptides for immunotherapy that are presented by MHC class II antigens, because there is a certain level of promiscuity in MHC class II-peptide binding and presentation to the immune system. At the same time, MHC class II presented peptides are required for activation of not only cytotoxic cells but also CD4+ve memory T cells. MHC class II mediated immunogenic response is therefore needed for a robust, offer long term immunogenicity for greater effectiveness in tumor protection. These problems can be addressed by: having a reliable peptide-MHC predicting algorithm and having a reliable system for assaying and validating the peptide-MHC interaction and immunogenicity. Therefore, in some embodiments, a highly efficient and immunogenic cancer vaccine may be produced by identifying candidate mutations in neoplasias/tumors which are present at the DNA level in tumor but not in matched germline samples from a high proportion of subjects having cancer; analyzing the identified mutations with one or more peptide-MHC binding prediction algorithms to identify which MHC (human leukocytic antigen or HLA in case of humans) bind to a high proportion of patient HLA alleles; and synthesizing the plurality of neoantigenic peptides selected from the sets of all neoantigen peptides and predicted binding peptides for use in a cancer vaccine or immunogenic composition suitable for treating a high proportion of subjects having cancer.

For example, translating peptide sequencing information into a therapeutic vaccine can include prediction of mutated peptides that can bind to HLA peptides of a high proportion of individuals. Efficiently choosing which particular mutations to utilize as immunogen requires the ability to predict which mutated peptides would efficiently bind to a high proportion of patient's HLA alleles. Recently, neural network based learning approaches with validated binding and non-binding peptides have advanced the accuracy of prediction algorithms for the major HLA-A and -B alleles. However, although using advanced neural network-based algorithms has helped to encode HLA-peptide binding rules, several factors limit the power to predict peptides presented on HLA alleles.

For example, translating peptide sequencing information into a therapeutic vaccine can include formulating the drug as a multi-epitope vaccine of long peptides. Targeting as many mutated epitopes as practically possible takes advantage of the enormous capacity of the immune system, prevents the opportunity for immunological escape by down-modulation of an immune targeted gene product, and compensates for the known inaccuracy of epitope prediction approaches. Synthetic peptides provide a useful means to prepare multiple immunogens efficiently and to rapidly translate identification of mutant epitopes to an effective vaccine. Peptides can be readily synthesized chemically and easily purified utilizing reagents free of contaminating bacteria or animal substances. The small size allows a clear focus on the mutated region of the protein and also reduces irrelevant antigenic competition from other components (unmutated protein or viral vector antigens).

For example, translating peptide sequencing information into a therapeutic vaccine can include a combination with a strong vaccine adjuvant. Effective vaccines can require a strong adjuvant to initiate an immune response. For example, poly-ICLC, an agonist of TLR3 and the RNA helicase-domains of MDA5 and RIG3, has shown several desirable properties for a vaccine adjuvant. These properties include the induction of local and systemic activation of immune cells in vivo, production of stimulatory chemokines and cytokines, and stimulation of antigen-presentation by DCs. Furthermore, poly-ICLC can induce durable CD4+ and CD8+ responses in humans. Importantly, striking similarities in the upregulation of transcriptional and signal transduction pathways were seen in subjects vaccinated with poly-ICLC and in volunteers who had received the highly effective, replication-competent yellow fever vaccine. Furthermore, >90% of ovarian carcinoma patients immunized with poly-ICLC in combination with a NYESO-1 peptide vaccine (in addition to Montanide) showed induction of CD4+ and CD8+ T cell, as well as antibody responses to the peptide in a recent phase 1 study. At the same time, poly-ICLC has been extensively tested in more than 25 clinical trials to date and exhibited a relatively benign toxicity profile.

In some embodiments, immunogenic peptides can be identified from cells from a subject with a disease or condition. In some embodiments, immunogenic peptides can be specific to a subject with a disease or condition. In some embodiments, immunogenic peptides can bind to an HLA that is matched to an HLA haplotype of a subject with a disease or condition.

In some embodiments, a library of peptides can be expressed in the cells. In some embodiments, the cells comprise the peptides to be identified or characterized. In some embodiments, the peptides to be identified or characterized are endogenous peptides. In some embodiments, the peptides are exogenous peptides. For example, the peptides to be identified or characterized can be expressed from a plurality of sequences encoding a library of peptides.

Prior to disclosure of the instant specification, the majority of LC-MS/MS studies of the HLA peptidome have used cells expressing multiple HLA peptides, which requires peptides to be assigned to 1 of up to 6 HLA class I alleles using pre-existing bioinformatic predictors or “deconvolution” (Bassani-Sternberg and Gfeller, 2016). Thus, peptides that do not closely match known motifs could not confidently be reported as binders to a given HLA allele.

Provided herein are methods of prediction of peptides, such as mutated peptides, that can bind to HLA peptides of individuals. In some embodiments, the application provides methods of identifying from a given set of antigen comprising peptides the most suitable peptides for preparing an immunogenic composition for a subject, said method comprising selecting from a given set of peptides the plurality of peptides capable of binding an HLA protein of the subject, wherein said ability to bind an HLA protein is determined by analyzing the sequence of peptides with a machine which has been trained with peptide sequence databases corresponding to the specific HLA-binding peptides for each of the HLA-alleles of said subject. Provided herein are methods of identifying from a given set of antigen comprising peptides the most suitable peptides for preparing an immunogenic composition for a subject, said method comprising selecting from a given set of peptides the plurality of peptides determined as capable of binding an HLA protein of the subject, ability to bind an HLA protein is determined by analyzing the sequence of peptides with a machine which has been trained with a peptide sequence database obtained by carrying out the methods described herein above. Thus, in some embodiments, the present disclosure provides methods of identifying a plurality of subject-specific peptides for preparing a subject-specific immunogenic composition, wherein the subject has a tumor and the subject-specific peptides are specific to the subject and the subject's tumor, said method comprising: sequencing a sample of the subject's tumor and a non-tumor sample of the subject; determining based on the nucleic acid sequencing: non-silent mutations present in the genome of cancer cells of the subject but not in normal tissue from the subject, and the HLA genotype of the subject; and selecting from the identified non-silent mutations the plurality of subject-specific peptides, each having a different tumor epitope that is specific to the tumor of the subject and each being identified as capable of binding an HLA protein of the subject, as determined by analyzing the sequence of peptides derived from the non-silent mutations in the methods for predicting HLA binding described herein.

In some embodiments, disclosed herein, is a method of characterizing HLA-peptide complexes specific to an individual.

In some embodiments, a method of characterizing HLA-peptide complexes specific to an individual is used to develop an immunotherapeutic in an individual in need thereof, such as a subject with a condition or disease.

Provided herein is a method of providing an anti-tumor immunity in a mammal comprising administering to the mammal a polynucleic acid comprising a sequence encoding a peptide identified according to a method described. Provided herein is a method of providing an anti-tumor immunity in a mammal comprising administering to the mammal an effective amount of a peptide with a sequence of a peptide identified according to a method described herein. Provided herein is a method of providing an anti-tumor immunity in a mammal comprising administering to the mammal a cell comprising a peptide comprising the sequence of a peptide identified according to a method described herein. Provided herein is a method of providing an anti-tumor immunity in a mammal comprising administering to the mammal a cell comprising a polynucleic acid comprising a sequence encoding a peptide comprising the sequence of peptide identified according to a method described herein. In some embodiments, the cell presents the peptide as an HLA-peptide complex.

Provided herein is a method of treating a disease or disorder in a subject, the method comprising administering to the subject a polynucleic acid comprising a sequence encoding a peptide identified according to a method described herein. Provided herein is a method of treating a disease or disorder in a subject, the method comprising administering to the subject an effective amount of a peptide comprising the sequence of a peptide identified according to a method described herein. Provided herein is a method of treating a disease or disorder in a subject, the method comprising administering to the subject a cell comprising a peptide comprising the sequence of a peptide identified according to a method described herein. Provided herein is a method of treating a disease or disorder in a subject, the method comprising administering to the subject a cell comprising a polynucleic acid comprising a sequence encoding a peptide comprising the sequence of a peptide identified according to a method described herein. In some embodiments, the disease or disorder is cancer. In some embodiments, the method further comprises administering an immune checkpoint inhibitor to the subject.

Disclosed herein, in some embodiments, are methods of developing an immunotherapeutic for an individual in need thereof by characterizing HLA-peptide complexes comprising: a) providing a population of cells derived from the individual in need thereof wherein one or more cells of the population of cells comprise a polynucleic acid comprising a sequence encoding an affinity acceptor tagged HLA class I or HLA class II allele, wherein the sequence encoding an affinity acceptor tagged HLA comprises: i) a sequence encoding a recombinant HLA class I or HLA class II allele operatively linked to ii) a sequence encoding an affinity acceptor peptide; b) expressing the affinity acceptor tagged HLA in at least one cell of the one or more cells of the population of cells, thereby forming affinity acceptor tagged HLA-peptide complexes in the at least one cell; c) enriching for the affinity acceptor tagged HLA-peptide complexes, characterizing HLA-peptide complexes specific to the individual in need thereof; and d) developing the immunotherapeutic based on an HLA-peptide complex specific to the individual in need thereof; wherein the individual has a disease or condition.

In some embodiments, the immunotherapeutic is a nucleic acid or a peptide therapeutic.

In some embodiments, the method comprises introducing one or more peptides to the population of cells. In some embodiments, the method comprises contacting the population of cells with the one or more peptides or expressing the one or more peptides in the population of cells. In some embodiments, the method comprises contacting the population of cells with one or more nucleic acids encoding the one or more peptides.

In some embodiments, the method comprises developing an immunotherapeutic based on peptides identified in connection with the patient-specific HLAs. In some embodiments, the population of cells is derived from the individual in need thereof.

In some embodiments, the method comprises expressing a library of peptides in the population of cells. In some embodiments, the method comprises expressing a library of affinity acceptor tagged HLA-peptide complexes. In some embodiments, the library comprises a library of peptides associated with the disease or condition. In some embodiments, the disease or condition is cancer or an infection with an infectious agent or an autoimmune disease. In some embodiments, the method comprises introducing the infectious agent or portions thereof into one or more cells of the population of cells. In some embodiments, the method comprises characterizing one or more peptides from the HLA-peptide complexes specific to the individual in need thereof, optionally wherein the peptides are from one or more target proteins of the infectious agent or the autoimmune disease. In some embodiments, the method comprises characterizing one or more regions of the peptides from the one or more target proteins of the infectious agent or autoimmune disease. In some embodiments, the method comprises identifying peptides from the HLA-peptide complexes derived from an infectious agent or an autoimmune disease.

In some embodiments, the infectious agent is a pathogen. In some embodiments, the pathogen is a virus, bacteria, or a parasite.

In some embodiments, the virus is selected from the group consisting of: BK virus (BKV), Dengue viruses (DENV-1, DENV-2, DENV-3, DENV-4, DENV-5), cytomegalovirus (CMV), Hepatitis B virus (HBV), Hepatitis C virus (HCV), Epstein-Barr virus (EBV), an adenovirus, human immunodeficiency virus (HIV), human T cell lymphotrophic virus (HTLV-1), an influenza virus, RSV, HPV, rabies, mumps rubella virus, poliovirus, yellow fever, hepatitis A, hepatitis B, Rotavirus, varicella virus, human papillomavirus (HPV), smallpox, zoster, and combinations thereof.

In some embodiments, the bacteria is selected from the group consisting of: Klebsiella spp., Tropheryma whipplei, Mycobacterium leprae, Mycobacterium lepromatosis, and Mycobacterium tuberculosis. In some embodiments, the bacteria is selected from the group consisting of: typhoid, pneumococcal, meningococcal, haemophilus B, anthrax, tetanus toxoid, meningococcal group B, bcg, cholera, and combinations thereof.

In some embodiments, the parasite is a helminth or a protozoan. In some embodiments, the parasite is selected from the group consisting of Leishmania spp. (e.g. L. major, L. infantum, L. braziliensis, L. donovani, L. chagasi, L. mexicana), Plasmodium spp. (e.g. P. falciparum, P. vivax, P. ovale, P. malariae), Trypanosoma cruzi, Ascaris lumbricoides, Trichuris trichiura, Necator americanus, and Schistosoma spp. (S. mansoni, S. haematobium, S. japonicum).

In some embodiments, the immunotherapeutic is an engineered receptor. In some embodiments, the engineered receptor is a chimeric antigen receptor (CAR), a T cell receptor (TCR), or a B cell receptor (BCR), an adoptive T cell therapy (ACT), or a derivative thereof. In other aspects, the engineered receptor is a chimeric antigen receptor (CAR). In some aspects, the CAR is a first generation CAR. In other aspects, the CAR is a second generation CAR. In still other aspects, the CAR is a third generation CAR.

In some aspects, the CAR comprises an extracellular portion, a transmembrane portion, and an intracellular portion. In some aspects, the intracellular portion comprises at least one T cell co-stimulatory domain. In some aspects, the T cell co-stimulatory domain is selected from the group consisting of CD27, CD28, TNFRS9 (4-1BB), TNFRSF4 (OX40), TNFRSF8 (CD30), CD40LG (CD40L), ICOS, ITGB2 (LFA-1), CD2, CD7, KLRC2 (NKG2C), TNFRS18 (GITR), TNFRSF14 (HVEM), or any combination thereof.

In some aspects, the engineered receptor binds a target. In some aspects, the binding is specific to a peptide identified from the method of characterizing HLA-peptide complexes specific to an individual suffering from a disease or condition.

In some aspects, the immunotherapeutic is a cell as described in detail herein. In some aspects, the immunotherapeutic is a cell comprising a receptor that specifically binds a peptide identified from the method characterizing HLA-peptide complexes specific to an individual suffering from a disease or condition. In some aspects, the immunotherapeutic is a cell used in combination with the peptides/nucleic acids of this invention. In some embodiments, the cell is a patient cell. In some embodiments, the cell is a T cell. In some embodiments, the cell is tumor infiltrating lymphocyte.

In some aspects, a subject with a condition or disease is treated based on a T cell receptor repertoire of the subject. In some embodiments, an antigen vaccine is selected based on a T cell receptor repertoire of the subject. In some embodiments, a subject is treated with T cells expressing TCRs specific to an antigen or peptide identified using the methods described herein.

In some embodiments, a subject is treated with an antigen or peptide identified using the methods described herein specific to TCRs, e.g., subject specific TCRs. In some embodiments, a subject is treated with an antigen or peptide identified using the methods described herein specific to T cells expressing TCRs, e.g., subject specific TCRs. In some embodiments, a subject is treated with an antigen or peptide identified using the methods described herein specific to subject specific TCRs.

In some embodiments, an immunogenic antigen composition or vaccine is selected based on TCRs identified in a subject. In one embodiment, identifying a T cell repertoire and testing it in functional assays is used to determine an immunogenic composition or vaccine to be administered to a subject with a condition or disease. In some embodiments, the immunogenic composition is an antigen vaccine. In some embodiments, the antigen vaccine comprises subject specific antigen peptides. In some embodiments, antigen peptides to be included in an antigen vaccine are selected based on a quantification of subject specific TCRs that bind to the antigens.

In some embodiments, antigen peptides are selected based on a binding affinity of the peptide to a TCR. In some embodiments, the selecting is based on a combination of both the quantity and the binding affinity. For example, a TCR that binds strongly to an antigen in a functional assay but is not highly represented in a TCR repertoire can be a good candidate for an antigen vaccine because T cells expressing the TCR would be advantageously amplified.

In some embodiments, antigens are selected for administering to a subject based on binding to TCRs. In some embodiments, T cells, such as T cells from a subject with a disease or condition, can be expanded. Expanded T cells that express TCRs specific to an immunogenic antigen peptide identified using the method described herein can be administered back to a subject.

In some embodiments, suitable cells, e.g., PBMCs, are transduced or transfected with polynucleotides for expression of TCRs specific to an immunogenic antigen peptide identified using the method described herein and administered to a subject. T cells expressing TCRs specific to an immunogenic antigen peptide identified using the method described herein can be expanded and administered back to a subject. In some embodiments, T cells that express TCRs specific to an immunogenic antigen peptide identified using the method described herein that result in cytolytic activity when incubated with autologous diseased tissue can be expanded and administered to a subject. In some embodiments, T cells used in functional assays result in binding to an immunogenic antigen peptide identified using the method described herein can be expanded and administered to a subject. In some embodiments, TCRs that have been determined to bind to subject specific immunogenic antigen peptides identified using the method described herein can be expressed in T cells and administered to a subject.

The methods described herein can involve adoptive transfer of immune system cells, such as T cells, specific for selected antigens, such as tumor or pathogen associated antigens. Various strategies can be employed to genetically modify T cells by altering the specificity of the T cell receptor (TCR), for example by introducing new TCR α—and β-chains with specificity to an immunogenic antigen peptide identified using the method described herein (see, e.g., U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

Chimeric antigen receptors (CARs) can be used to generate immunoresponsive cells, such as T cells, specific for selected targets, such a immunogenic antigen peptides identified using the method described herein, with a wide variety of receptor chimera constructs (see, e.g., U.S. Pat. Nos. 5,843,728; 5,851,828; 5,912, 170; 6,004,811; 6,284,240; 6,392,013; 6,410,014; 6,753,162; 8,211,422; and, PCT Publication WO9215322). Alternative CAR constructs can be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signaling domains of either CD3ζ or FcRy or scFv-FcRy (see, e.g., U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain, e.g., scFv-CD28/OX40/4-1BB-CD3 (see, e.g., U.S. Pat. Nos. 8,911,993; 8,916,381; 8,975,071; 9,101,584; 9,102,760; 9,102,761). Third-generation CARs include a combination of costimulatory endodomains, such a CD3C-chain, CD97, GDI 1a-CD18, CD2, ICOS, CD27, CD154, CDS, OX40, 4-1BB, or CD28 signaling domains, e.g., scFv-CD28-4-1BB-CD3C or scFv-CD28-OX40-CD3Q (see, e.g., U.S. Pat. Nos. 8,906,682; 8,399,645; 5,686,281; PCT Publication No. WO2014134165; PCT Publication No.

WO2012079000). In some embodiments, costimulation can be coordinated by expressing CARs in antigen-specific T cells, chosen so as to be activated and expanded following, for example, interaction with antigen on professional antigen-presenting cells, with costimulation. Additional engineered receptors can be provided on the immunoresponsive cells, e.g., to improve targeting of a T cell attack and/or minimize side effects.

Alternative techniques can be used to transform target immunoresponsive cells, such as protoplast fusion, lipofection, transfection or electroporation. A wide variety of vectors can be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), can be used to introduce CARs, for example using 2nd generation antigen-specific CARs signaling through CD3ζ and either CD28 or CD137. Viral vectors can, for example, include vectors based on HIV, SV40, EBV, HSV or BPV.

Cells that are targeted for transformation can, for example, include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells can be differentiated. T cells expressing a desired CAR can, for example, be selected through co-culture with γ-irradiated activating and propagating cells (APC), which co-express the cancer antigen and co-stimulatory molecules. The engineered CAR T cells can be expanded, for example, by co-culture on APC in presence of soluble factors, such as IL-2 and IL-21. This expansion can, for example, be carried out so as to provide memory CAR T cells (which, for example, can be assayed by non-enzymatic digital array and/or multi-panel flow cytometry). In this way, CAR T cells that have specific cytotoxic activity against antigen-bearing tumors can be provided (optionally in conjunction with production of desired chemokines such as interferon-γ). CAR T cells of this kind can, for example, be used in animal models, for example to threaten tumor xenografts.

Approaches such as the foregoing can be adapted to provide methods of treating and/or increasing survival of a subject having a disease, such as a neoplasia or pathogenic infection, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction). Dosing in CAR T cell therapies can, for example, involve administration of from 106 to 109 cells/kg, with or without a course of lymphodepletion, for example with cyclophosphamide.

To guard against possible adverse reactions, engineered immunoresponsive cells can be equipped with a transgenic safety switch in the form of a transgene that renders the cells vulnerable to exposure to a specific signal. For example, the herpes simplex viral thymidine kinase (TK) gene can be used in this way, for example by introduction into allogeneic T lymphocytes used as donor lymphocyte infusions following stem cell transplantation. In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see, e.g., U.S. Patent Publication No. 20130071414; PCT Patent Publication WO2011146862; PCT Patent Publication WO201401 1987; PCT Patent Publication WO2013040371). In a further refinement of adoptive therapies, genome editing can be used to tailor immunoresponsive cells to alternative implementations, for example providing edited CAR T cells.

Cell therapy methods can also involve the ex vivo activation and expansion of T cells. In some embodiments, T cells can be activated before administering them to a subject in need thereof. Examples of these type of treatments include the use tumor infiltrating lymphocyte (TIL) cells (see U.S. Pat. No. 5,126,132), cytotoxic T cells (see U.S. Pat. Nos. 6,255,073; 5,846,827), expanded tumor draining lymph node cells (see U.S. Pat. No. 6,251,385), and various other lymphocyte preparations (see U.S. Pat. Nos. 6,194,207; 5,443,983; U.S. Pat. Nos. 6,040,177; 5,766,920).

An ex vivo activated T cell population can be in a state that maximally orchestrates an immune response to cancer, infectious diseases, or other disease states, e.g., an autoimmune disease state. For activation, at least two signals can be delivered to the T cells. The first signal is normally delivered through the T cell receptor (TCR) on the T cell surface. The TCR first signal is normally triggered upon interaction of the TCR with peptide antigens expressed in conjunction with an MHC complex on the surface of an antigen-presenting cell (APC). The second signal is normally delivered through co-stimulatory receptors on the surface of T cells. Co-stimulatory receptors are generally triggered by corresponding ligands or cytokines expressed on the surface of APCs.

It is contemplated that the T cells specific to immunogenic antigen peptides identified using the method described herein can be obtained and used in methods of treating or preventing disease. In this regard, the disclosure provides a method of treating or preventing a disease or condition in a subject, comprising administering to the subject a cell population comprising cells specific to immunogenic antigen peptides identified using the method described herein in an amount effective to treat or prevent the disease in the subject. In some embodiments, a method of treating or preventing a disease in a subject, comprises administering a cell population enriched for disease-reactive T cells to a subject in an amount effective to treat or prevent cancer in the mammal. The cells can be cells that are allogeneic or autologous to the subject.

The disclosure further provides a method of inducing a disease specific immune response in a subject, vaccinating against a disease, treating and/or alleviating a symptom of a disease in a subject by administering the subject an antigenic peptide or vaccine.

The peptide or composition of the disclosure can be administered in an amount sufficient to induce a CTL response. An antigenic peptide or vaccine composition can be administered alone or in combination with other therapeutic agents. Exemplary therapeutic agents include, but are not limited to, a chemotherapeutic or biotherapeutic agent, radiation, or immunotherapy. Any suitable therapeutic treatment for a particular disease can be administered. Examples of chemotherapeutic and biotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochloroperazine, rituximab, tamoxifen, taxol, topotecan hydrochloride, trastuzumab, vinblastine, vincristine and vinorelbine tartrate. In addition, the subject can be further administered an anti-immunosuppressive or immunostimulatory agent. For example, the subject can be further administered an anti-CTLA antibody or anti-PD−1 or anti-PD-L1.

The amount of each peptide to be included in a vaccine composition and the dosing regimen can be determined by one skilled in the art. For example, a peptide or its variant can be prepared for intravenous (i.v.) injection, sub-cutaneous (s.c.) injection, intradermal (i.d.) injection, intraperitoneal (i.p.) injection, intramuscular (i.m.) injection. Exemplary methods of peptide injection include s.c, i.d., i.p., i.m., and i.v. Exemplary methods of DNA injection include i.d., i.m., s.c, i.p. and i.v. Other methods of administration of the vaccine composition are known to those skilled in the art.

A pharmaceutical composition can be compiled such that the selection, number and/or amount of peptides present in the composition is/are disease and/or patient-specific. For example, the exact selection of peptides can be guided by expression patterns of the parent proteins in a given tissue to avoid side effects. The selection can be dependent on the specific type of disease, the status of the disease, earlier treatment regimens, the immune status of the patient, and the HLA-haplotype of the patient. Furthermore, the vaccine according to the present disclosure can contain individualized components, according to personal needs of the particular patient. Examples include varying the amounts of peptides according to the expression of the related antigen in the particular patient, unwanted side-effects due to personal allergies or other treatments, and adjustments for secondary treatments following a first round or scheme of treatment.

### Computer Control Systems

The present disclosure provides computer control systems that are programmed to implement methods of the disclosure. A computer system that is programmed or otherwise configured to train a machine-learning HLA-peptide presentation prediction model can be used. The computer system can regulate various aspects of the present disclosure, such as, for example, inputting amino acid position information, transferring imputed information into datasets, and generating a trained algorithm with the datasets. The computer system can be an user electronic device or a remote computer system. The electronic device can be a mobile electronic device.

The computer system can include a central processing unit (CPU, also “processor” and “computer processor” herein), which can be a single core or multi core processor, either through sequential processing or parallel processing. The computer system also includes a memory unit or device (e.g., random-access memory, read-only memory, flash memory), a storage unit (e.g., hard disk), a communication interface (e.g., network adapter) for communicating with one or more other systems, and peripheral devices, either external or internal or both, such as a printer, monitor, USB drive and/or CD-ROM drive. The memory, storage unit, interface and peripheral devices are in communication with the CPU through a communication bus (solid lines), such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable a peer-to-peer network that supports distributed computing. The network, in some cases with the aid of the computer system, can implement a client-server structure, which may enable devices coupled to the computer system to behave as a client or a server.

The CPU can execute a sequence of machine-readable instructions, which can be embodied in a program or software. The instructions may be stored in memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs. The computer system in some cases can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system or user. Examples of remote computer systems include personal computers (e.g., portable PC), slate or tablet PC's (e.g., Apple®iPad, Samsung® Galaxy Tab), telephones, Smart phones (e.g., Apple®iPhone, Android-enabled device, Blackberry®), or personal digital assistants. The user can access the computer system via the network.

Methods as described herein can be implemented by way of machine (e.g., computer processor) executable code stored on an electronic storage location of the computer system, such as, for example, in memory or a data storage unit. The machine executable or machine readable code can be provided in the form of software. During use, the code can be executed by the processor. In some cases, the code can be retrieved from the storage unit and stored in memory for ready access by the processor. In some situations, the storage unit can be precluded, and machine-executable instructions are stored in memory.

The code can be pre-compiled and configured for use with a machine having a processer adapted to execute the code, or it can be compiled during runtime. The code can be supplied in a programming language that can be selected to enable the code to execute in a pre-compiled or as-compiled fashion.

Aspects of the systems and methods provided herein, such as the computer system (1001), can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on a storage unit, such as a hard disk, or in memory (e.g., read-only memory, random-access memory, flash memory). “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, probability that one or more proteins encoded by a class II MHC allele of a cancer cell of the subject will present a given sequence of a peptide sequence identified. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, input amino acid position information, transfer imputed information into datasets, and generate a trained algorithm with the datasets.

### EXAMPLES

The example(s) provided below are for illustrative purposes only and do not limit the scope of the claims provided herein.

### Example 1. Validation of Predicted Neoantigens in Patient Derived Material by Targeted Mass Spectrometry

Immunotherapy has been shown to be effective against cancers with a high tumor mutation burden. While treatment with immune checkpoint blockades can result in durable remission, this outcome only occurs in about 20% of patients. More recently, in an effort to increase patient response rates, personalized cancer vaccines have been used to direct the immune system towards neoantigens—tumor mutations that are presented to the immune system on class I HLA complexes. Due to the highly polymorphic nature of class I HLA molecules and their different binding preferences, specialized machine learning algorithms have been developed to predict which neoantigens could bind to patient HLA molecules. At the core of the neoantigen platform is RECON®, a neural network algorithm trained on mono-allelic mass spectrometry data that predicts and selects therapeutically relevant targets to yield HLA presented neoantigens in patients. While RECON® has been thoroughly tested and validated with mass spectrometry samples generated in vitro, predicted neoantigen presentation has not been validated in a bonafide manner on clinical samples. Here, MS validation of predicted neoantigens from PDX models is demonstrated. In order to target a large number of predicted epitopes with a high degree of sensitivity, IS-PRM was deployed using an isotope labeling approach that avoids false positive signals from residual light material in the synthetic peptides. Furthermore, combinations of isotope labels with IS-PRM and pMHC spike-ins were leveraged to enable the absolute quantification of predicted neoantigens, and the most abundant epitopes are shown to elicit the strongest functional T cell response. Together, these data show that RECON®-predicted HLA-I epitopes are indeed presented to the immune system in clinical samples.

### Example 2. MS-Based Prediction Algorithm

RECON® is a neural network algorithm that was trained on high-quality mono-allelic HLA immunopeptidome data generated by MS. The accuracy of HLA-I ligand predictions by RECON® are improved from mono-allelic data (FIG. 1A) and ovarian tumors profiled by MS from Schuster et al. PNAS. 2017 (FIG. 1B) compared to the publicly available netMHCpan prediction tool. PPV=fraction of top n ranked peptides that were hits given n hits and 5000n decoys. RECON® provides a presentation score incorporating gene expression, binding prediction and peptide cleavability (FIG. 1C).

### Example 3. Patient Derived Xenografts for Targeted MS

Tumor tissue was obtained from core needle biopsies from two patients with advanced metastatic melanoma prior to receiving a personalized neoantigen vaccine (Ott et al., Cell 2020).

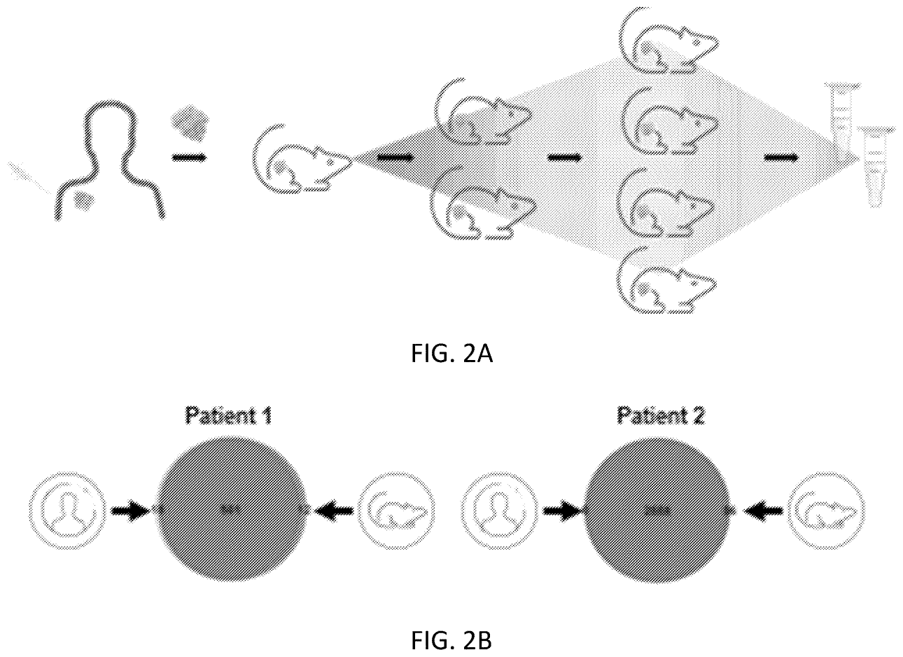

Tissue was engrafted and grown in immunocompromised mice before tumors were harvested and dissociated into single-cell suspensions (FIG. 2A). Next generation sequencing (NGS) was performed on the initial tumor biopsies and patient-derived xenograft (PDX) material from both patients and high sequence overlap of the non-synonymous mutations was observed (FIG. 2B). RECON® was used to generate a list of 123 and 136 epitopes with the highest RECON® presentation scores from patients 1 and 2, respectively, for targeted mass spectrometry. Table 1 shows the HLA alleles present in each patient. HLA-B*35:12 for Patient 1 is not supported by the current version of RECON®.

TABLE 1
Patient HLA alleles

| Patient 1 | Patient 2 |
|---|---|
| A*02:01 | A*03:01 |
| A*02:01 | A*24:02 |
| B*07:02 | B*14:02 |
| B*35:12 | B*38:01 |
| C*07:02 | C*08:02 |
| C*04:01 | C*12:03 |

### Example 4. Validation of Predicted Neoantigens Using Internal Standard Triggered Parallel Reaction Monitoring

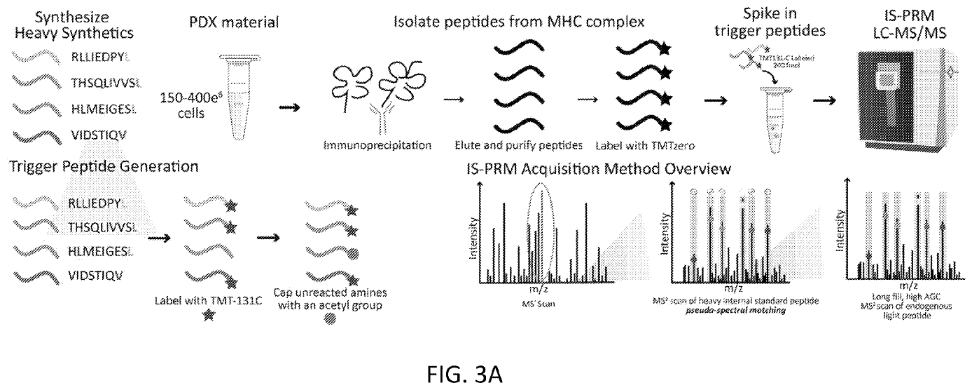

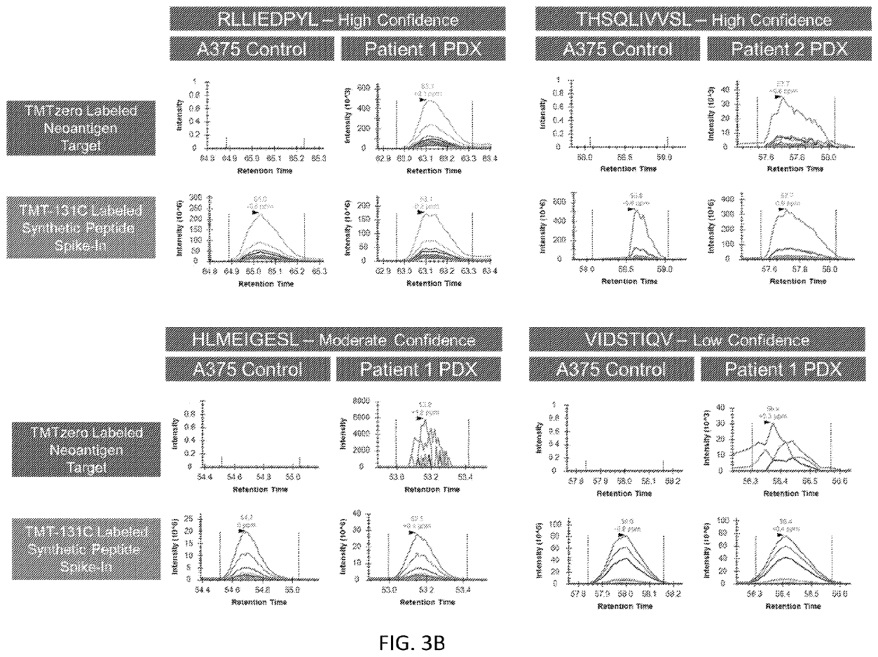

As shown in FIG. 3A (workflow for IS-PRM), endogenous peptides were isolated from PDX material using HLA-A*02:01 (patient 1) or pan HLA-I (patient 2) immunoprecipitation and acid elution. Peptides were desalted and labeled with TMTzero using standard protocols.

Synthetic peptides predicted peptide targets were synthesized in house for use as trigger peptides. Prior to IS-PRD analysis, TMT131C-labeled trigger peptides were spiked into the samples.

Table 2 shows the MS-identified neoantigens for both patients. Example Skyline chromatograms are shown for select peptides and compared to samples from A375 cells, an irrelevant melanoma cell line (FIG. 3).

TABLE 2
MS-Identified neoantigens from PDX
tumor material from patients 1 and 2.
Patient 1

| | | |
|---|---|---|
| Sequence | Confidence | Allele |
| RLLIEDPYL (SEQ ID NO: 10) | High | A*02:01 |
| NLPGFLPAL (SEQ ID NO: 11) | High | A*02:01 |
| HSAINEVVT (SEQ ID NO: 12) | High | A*02:01 |
| RLAQGSSAV (SEQ ID NO: 13) | High | A*02:01 |
| HLMEIGESL (SEQ ID NO: 14) | Moderate | A*02:01 |
| VIDSTIQSV (SEQ ID NO: 15) | Low | A*02:01 |
| HLYSGAVTI (SEQ ID NO: 16) | Low | A*02:01 |

Patient 2

| | | |
|---|---|---|
| MRIATPLLM (SEQ ID NO: 17) | High | B*14:02; |
| | | B*38:01 |
| YHGDDGHLF (SEQ ID NO: 18) | High | A*24:02; |
| | | B*38:01 |
| THSQLIVVSL (SEQ ID NO: 19) | High | B*38:01 |
| ILDVNVNL (SEQ ID NO: 20) | High | C*08:01; |
| | | C*12:03 |
| VAFGHLLAI (SEQ ID NO: 21) | High | C*12:03 |
| VHDAAQEGF (SEQ ID NO: 22) | High | B*38:01 |
| EHVPPYDVVL (SEQ ID NO: 23) | High | B*38:01 |
| QYQKVLVLF (SEQ ID NO: 24) | High | A*24:02 |
| SHQSFQHLL (SEQ ID NO: 25) | Low | B*38:01 |
| RVPFAFPLHR (SEQ ID NO: 26) | Low | A*03:01 |
| SYVTSHQGF (SEQ ID NO: 27) | Low | A*24:02 |
| MLFPTSAQK (SEQ ID NO: 28) | Low | A*03:01 |

### Example 5. RECON© Presentation Scores Across Epitopes Targeted by MS

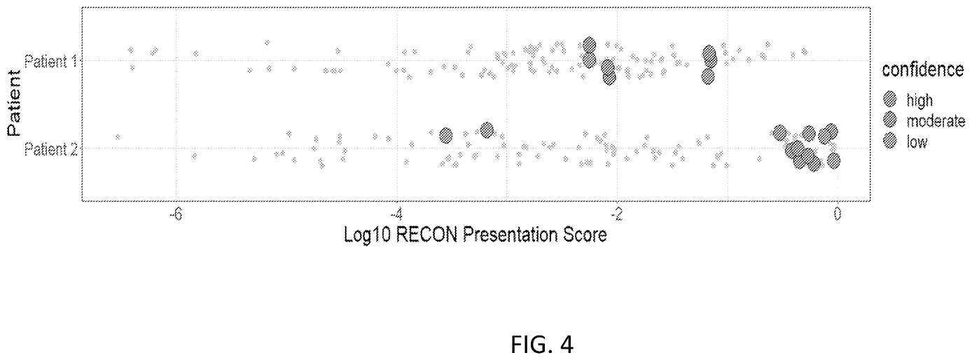

Seven and twelve neoantigens were successfully validated by IS-PRM from PDX material derived from patients 1 and 2, respectively. The plot in FIG. 4 shows RECON® presentation scores across all peptides targeted by MS with MS-detected neoantigens as indicated. MS-observed neoantigens generally have higher RECON® presentation scores.

### Example 6. Quantification of Predicted Neoantigens

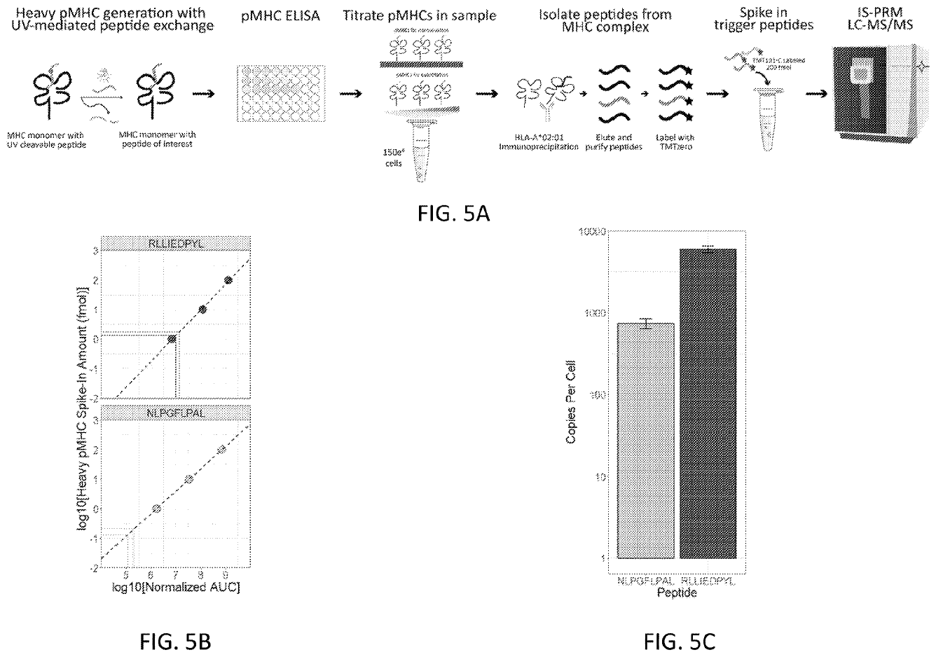

FIG. 5A shows an exemplary workflow and quantification method (adapted from Stopfer et al, PNAS 2021). A multichannel IS-PRM scheme (FIG. 5A and Table 3) was used to acquire absolute quantification of epitopes in PDX material derived from patient 1. Heavy isotopically labeled peptides were exchanged onto A*02:01 monomers and spiked directly into the cell lysate prior to immunoprecipitation of HLA-A*02:01. Samples were labeled with TMTzero, and TMT131C heavy synthetic peptides were added before analysis to serve as triggers for IS-PRM acquisition.

TABLE 3
Isotope labeling scheme for IS-PRM acquisition

| | | TMT | Peptide | Offset from |
|---|---|---|---|---|
| Purpose | Channel | Tag | Label | Target (Da) |
| Endogenous | Target | TMTzero | Light | 0 |
| Target | | | | |
| pMHC | Target | TMTzero | Heavy AA | +6 to +10 |
| Calibration | | | | |
| On-column | Trigger | TMT131C | Heavy AA | +11 to +15 |
| Spike-in | | | | |

Copies per cell for two neoantigens were successfully quantified (FIGS. 5A and 5B and Table 4). Four neoantigens were below the limit of quantification and could not be accurately quantified (Table 4). Peptide HSAINEVVT (SEQ ID NO: 29) could not be UV exchanged and showed no binding in a competitive binding assay (Table 5). Absolute quantification of peptide HLMEIGESL (SEQ ID NO: 14) was not possible due to variable oxidation of the methionine residue.

TABLE 4
Absolute Quantification of A*02:01
epitopes from Patient 1

| | Copies | |
|---|---|---|
| Sequence | per Cell | Allele |
| RLLIEDPYL (SEQ ID NO: 10) | 5.9 | A*02:01 |
| NLPGFLPAL (SEQ ID NO: 11) | 0.7 | A*02:01 |
| RLAQGSSAV (SEQ ID NO: 13) | <LOQ | A*02:01 |
| VIDSTIQSV (SEQ ID NO: 15) | <LOQ | A*02:01 |
| HLYSGAVTI (SEQ ID NO: 16) | <LOQ | A*02:01 |
| HLMEIGESL (SEQ ID NO: 14) | N/A | A*02:01 |
| | (Met | |
| | Oxidation) | |
| HSAINEVVT (SEQ ID NO: 29) | No Exchange | A*02:01 |

TABLE 5
IC50 values for MS-observed neoantigens
determined in a competitive binding
against a FITC labelled HLA-A*02:01 probe

| Sequence | IC₅₀ (nM) | Allele |
|---|---|---|
| RLLIEDPYL (SEQ ID NO: 10) | 235.3 | A*02:01 |
| NLPGFLPAL (SEQ ID NO: 11) | 5.074 | A*02:01 |
| RLAQGSSAV (SEQ ID NO: 13) | 966.3 | A*02:01 |
| HLMEIGESL (SEQ ID NO: 14) | 46.77 | A*02:01 |
| VIDSTIQSV (SEQ ID NO: 15) | 13.68 | A*02:01 |
| HLYSGAVTI (SEQ ID NO: 16) | 6.119 | A*02:01 |
| HSAINEVVT (SEQ ID NO: 29) | No | A*02:01 |
| | Binding | |

### Immune Monitoring Correlates

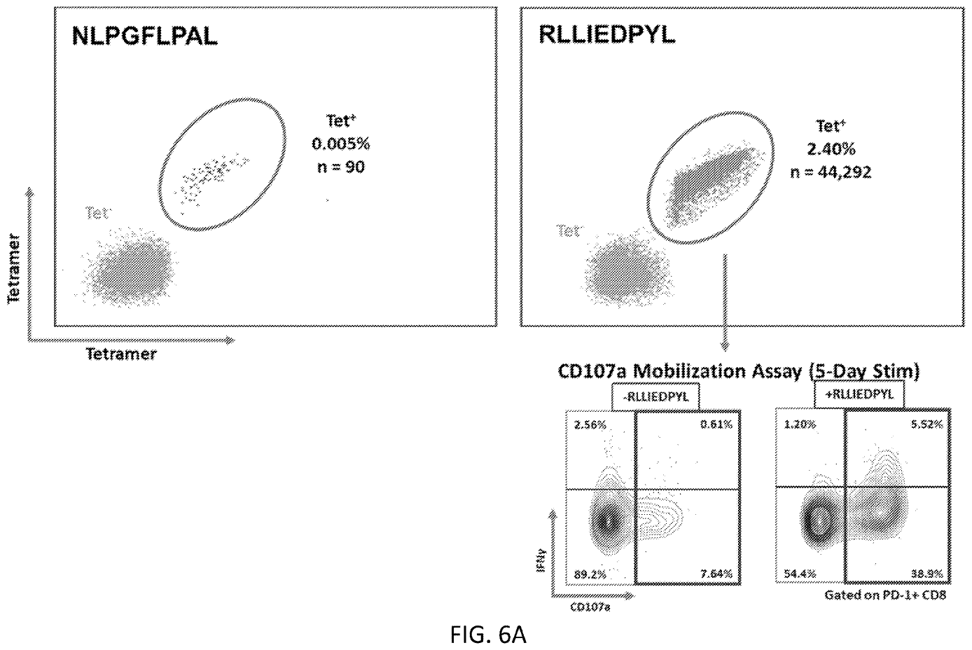

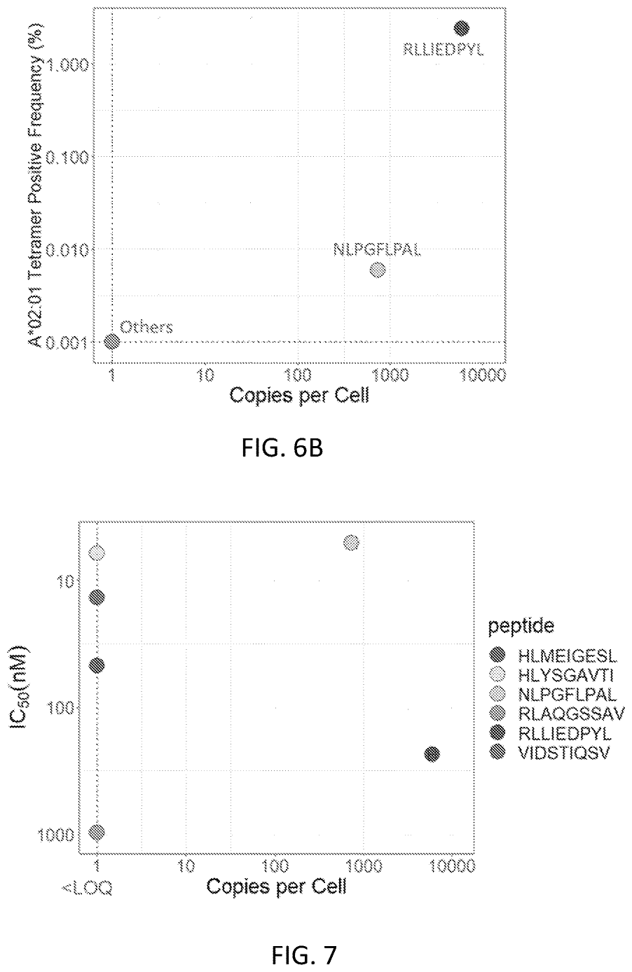

A*02:01 tetramer staining of PBMCs derived from Patient 1 reveals that the most highly presented epitope (RLLIEDPYL (SEQ ID NO: 10)) with the most copies per cell also results in the most frequent tetramer positive T cell population of all the epitopes tested (FIG. 6A and FIG. 6B). These neoantigen-specific T cells demonstrate cytotoxic potential as seen by increased CD107a+ and IFNγ+ subpopulations in the presence of the epitope.

### Binding Affinity Correlates

A competitive binding assay with a FITC labelled HLA-A*0201 probe was used to determine the binding affinities of MS-observed neoantigens from Patient 1 (Table 5, FIG. 7). No correlation between the abundance of presented epitopes and measured binding affinity to HLA-A*02:01 was observed.

### Example 7. Clinical Outcome

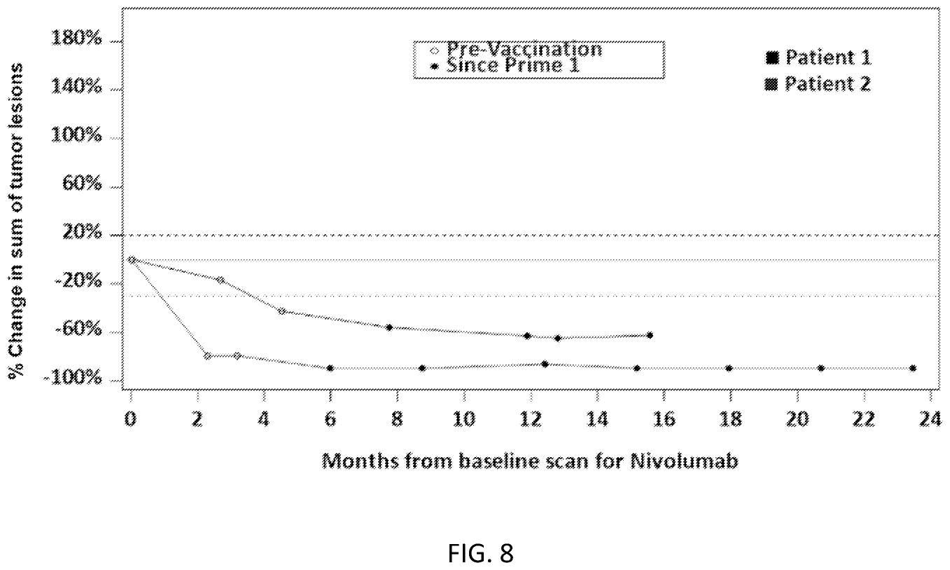

As shown in FIG. 8, both patients from which the PDX material was derived were classified as achieving durable clinical benefit from checkpoint blockade inhibition and personalized neoantigen vaccination according to RECIST criteria (Ott et al., Cell. 2020).

In this study, we use targeted mass spectrometry to validate the presentation of RECON® predicted neoantigens in clinically-derived patient material. Absolute quantification to yield copies per cell of presented epitopes on patient tumor material was performed with an isotope encoding scheme. By comparing the absolute epitope copy-per-cell number to T cell reactivity in the patient's peripheral blood, we observe that the most abundant epitopes also generate the most neoantigen-reactive T cells tested in the patient.

1-27. (canceled)

28. A method of identifying peptide sequences as being presented by at least one of one or more proteins encoded by an HLA allele of a cell of the subject comprising:
(a) inputting amino acid sequence information of a set of candidate peptide sequences expressed by cancer cells of a single human subject, using a computer processor, into a trained machine learning HLA-peptide presentation prediction model, to generate a plurality of presentation predictions, wherein each presentation prediction of the plurality of presentation predictions is indicative of a presentation likelihood that a peptide sequence of the set of candidate peptide sequences is presented by an MHC protein of the single human subject; wherein the trained machine learning HLA-peptide presentation prediction model comprises:
(i) a plurality of parameters, wherein the plurality of parameters are based on training data from training cells expressing an MHC protein, wherein the training data comprises a plurality of training peptide sequences and epitope presentation quantification information, wherein the epitope presentation quantification information comprises the amount of one or more of the training peptide sequences of the plurality that was presented by an MHC protein; and
(ii) a function representing a relation between the amino acid sequence information received as input and the presentation likelihood generated as an output based on the amino acid sequence information and the plurality of parameters; and
(b) identifying, based at least on the plurality of presentation predictions, a peptide sequence of the plurality of peptide sequences of the set of candidate peptide sequences as being presented by at least one of the one or more proteins encoded by an HLA allele of a cell of the subject.

29. The method of claim 28, further comprising selecting, based at least on the plurality of presentation predictions, a subset of peptide sequences of the set of candidate peptide sequences to generate a set of selected peptide sequences.

30. The method of claim 28, further comprising administering to the single human subject a pharmaceutical composition comprising:
(i) a polypeptide with the peptide sequence,
(ii) a polynucleotide encoding the polypeptide of (i);
(iii) APCs comprising (i) or (ii), or
(iv) T cells comprising a T cell receptor (TCR) specific for an MHC protein of the single human subject in complex with one or more of the peptide sequences identified in (b).

31. The method of claim 28, wherein the plurality of parameters are based on training data from training cells expressing an MHC protein of the single human subject, wherein each training peptide sequence of the plurality is associated with an MHC protein.

32. The method of claim 31, wherein the training data comprises an identity of the MHC protein associated with each training peptide sequence of the plurality or an observation by mass spectrometry that one or more of the training peptide sequences of the plurality was presented by an MHC protein.

33. The method of claim 28, wherein the MHC protein of the single human subject is a class I MHC protein.

34. The method of claim 28, wherein the plurality of candidate peptide sequences expressed by cancer cells of a single human subject are identified by comparing whole genome or whole exome sequence information from the cancer cells of the single human subject to whole genome or whole exome sequence information from non-cancer cells of the single human subject, and identifying nucleic acid sequences unique to the cancer cells and not present in the non-cancer cells.

35. The method of claim 28, wherein each candidate sequence of the plurality of candidate peptide sequences comprises a cancer specific mutation.

36. The method of claim 28, wherein the trained machine learning HLA-peptide presentation prediction model having a peptide presentation prediction value (PPV) of at least 0.2 according to a presentation PPV determination method.

37. The method of claim 28, wherein the presentation PPV determination method comprises inputting amino acid sequence information of a plurality of test peptide sequences into the trained machine learning HLA-peptide presentation prediction model to generate a plurality of test presentation predictions, each test presentation prediction indicative of a likelihood that the one or more proteins encoded by an HLA allele can present a given test peptide sequence of the plurality of test peptide sequences, wherein the plurality of test peptide sequences comprises at least 500 test peptide sequences comprising:
(i) at least one hit peptide sequence identified by mass spectrometry to be presented by an HLA protein expressed in cells, and
(ii) at least 499 decoy peptide sequences contained within a protein encoded by a genome of an organism, wherein the organism and the subject are the same species.

38. The method of claim 37, wherein the plurality of test peptide sequences comprises a ratio of 1:499 of the at least one hit peptide sequence to the at least 499 decoy peptide sequences and a top 0.2% of the plurality of test peptide sequences are predicted to be presented by the HLA protein expressed in cells by the trained machine learning HLA-peptide presentation prediction model; wherein (i) the at least one hit peptide sequence comprises at least 10 hit peptide sequences, and (ii) the at least 499 decoy peptide sequences comprise at least 4,990 decoy peptide sequences.

39. The method of claim 28, wherein the amount of one or more of the training peptide sequences of the plurality that was presented by an MHC protein comprises number of copies of the one or more of the training peptide sequences of the plurality that was presented by an MHC protein or number of copies per cell of the one or more of the training peptide sequences of the plurality that was presented by an MHC protein.

40. The method of claim 28, wherein the amount of one or more of the training peptide sequences of the plurality that was presented by an MHC protein comprises the absolute quantity, the number of molecules, density, concentration, absolute quantity per cell, the number of molecules per cell, density per cell, or concentration in a cell of the one or more of the training peptide sequences of the plurality that was presented by an MHC protein.

41. The method of claim 28, wherein the amount of one or more of the training peptide sequences of the plurality that was presented by an MHC protein is based on a number of mass spectrometry observances, spectral counting, area under the curve (AUC), intensity-based absolute quantification (iBAQ), label free quantification (LFQ), isotope dilution mass spectrometry, isobaric mass tagging, stable isotope labeling, and/or mass spectrometry peak intensity.

42. The method of claim 28, wherein the amount of one or more of the training peptide sequences of the plurality that was presented by an MHC protein is obtained from quantitative mass spectrometry.

43. The method of claim 28, wherein the epitope presentation quantification information is obtained from internal standard-parallel reaction monitoring (IS-PRM) mass spectrometry or obtained from a xenograft sample.

44. The method of claim 43, wherein the xenograft sample is a patient-derived xenograft (PDX) sample.

45. A method of selecting peptide sequences comprising:
(a) inputting amino acid sequence information of a set of candidate peptide sequences expressed by cancer cells of a single human subject, using a computer processor, into a trained machine learning HLA-peptide antigen-specific T cell prediction model, to generate a plurality of antigen-specific T cell predictions, wherein each antigen-specific T cell prediction of the plurality of antigen-specific T cell predictions is indicative of a likelihood that an MHC complex comprising an MHC protein of the single human subject and a peptide sequence of the set of candidate peptide sequences stimulates a T cell to be specific to a peptide sequence of the set of candidate peptide sequences; wherein the trained machine learning HLA-peptide antigen-specific T cell prediction model comprises:
(i) a plurality of parameters, wherein the plurality of parameters are based on training data from training cells expressing an MHC protein, wherein the training data comprises a plurality of training peptide sequences and epitope presentation quantification information, wherein the epitope presentation quantification information comprises the amount of one or more of the training peptide sequences of the plurality that was presented by an MHC protein; and
(ii) a function representing a relation between the amino acid sequence information received as input and the likelihood that a T cell specific to a peptide sequence of the set of candidate peptide sequences would be generated as an output based on the amino acid sequence information and the plurality of parameters; and
(b) selecting, based at least on the plurality of antigen-specific T cell predictions, a subset of peptide sequences of the set of candidate peptide sequences to generate a set of selected peptide sequences.

46. The method of claim 45, wherein each antigen-specific T cell prediction of the plurality of antigen-specific T cell predictions is indicative of the likelihood that the MHC complex comprising an MHC protein of the single human subject and a peptide sequence of the set of candidate peptide sequences stimulates a T cell to be cytotoxic or to be specific to a neoantigen peptide sequence of the set of candidate peptide sequences.

47. The method of claim 45, wherein the function is a function representing a relation between the amino acid sequence information received as input and the likelihood that a cytotoxic T cell or a T cell specific to a neoantigen peptide sequence of the set of candidate peptide sequences would be generated as an output based on the amino acid sequence information and the plurality of parameters.
